(19) United States
(12) Patent Application Publication
(10) Pub. No.: US 2007/0195309 A1
(43) Pub. Date: Aug. 23, 2007

(54) PHOTOCHROMIC PROBES

(75) Inventors: Gerard J. D. MARRIOTT, Madison, WI (US); Tomoyo SAKATA, Madison, WI (US); Yuling YAN, Honululu, WI (US)

Correspondence Address: GODFREY & KAHN, S.C., 780 N. WATER STREET, MILWAUKEE, WI, 53202, US

(73) Assignee: WISCONSIN ALUMNI RESEARCH FOUNDATION, Madison, WI (US)

(21) Appl. No.: 11/164,353

(22) Filed: Nov. 18, 2005

Related U.S. Application Data
(60) Provisional application No. 60/522,904, filed on Nov. 18, 2004.

(51) Int. Cl. C07D 209/96; C07D 265/28; G11C 5/02

(52) U.S. Cl. 356/51; 356/256; 436/92; 544/71; 548/409

(57) ABSTRACT

The present invention provides photochromic compounds and derivatives thereof as shown in claim 1 and methods of use of these compounds and derivatives. The present invention also provides photochromic optical probes capable of undergoing light directed reversible transition between a first state and a second state. The invention also teaches methods of determining and controlling reversible optical biomolecular interactions, for example binding of calcium in a subject.

### RELATED APPLICATION

The present application seeks priority from U.S. Provisional Patent Application No. 60/522,904, which is herein incorporated by reference for all purposes.

### STATEMENT REGARDING FEDERAL FUNDING

This invention was made with United States government support awarded by the following agency: NIH HL069970. The United States government has certain rights in this invention.

### TECHNICAL FIELD

The present invention provides methods and compounds for use as reversible optical switches and probes for studying and manipulating biomolecular interactions. Specifically these optical switches are based on the reversible optical chemistry of colorless spirobenzopyran (SP) and colored merocynanine (MC) states.

### BACKGROUND

Optical probes capable of specifically manipulating protein interactions and activities in complex environments¹⁻³ are useful for understanding cellular processes in terms of the reaction mechanisms and its underlying protein function.

A serious limitation, however of certain optical probes such as 2-nitrophenyl-based caged groups is that the photoisomerization reaction that leads to the activation of the protein is irreversible and they function as self-destructing, one-way, single-use, optical switches.

Further, Willner et al¹¹ have shown that although binding of certain conjugates, which are randomly labeled with multiple photochromes, is possible, however, these conjugates are polydisperse and spectroscopically complex.

Accordingly, the need exists to have stereoscopically simpler approaches to reversible, optical switching of biomolecular interactions. Further, the need exists for seeking activity that employs chemically and spectroscopically defined conjugates harboring a single and specifically labeled photochromic probe.

### SUMMARY OF THE INVENTION

The present invention generally provides compounds and methods for using reversible photochromic compounds as probes. In one embodiment, the present invention provides a compound or a derivative thereof selected from the group consisting of:

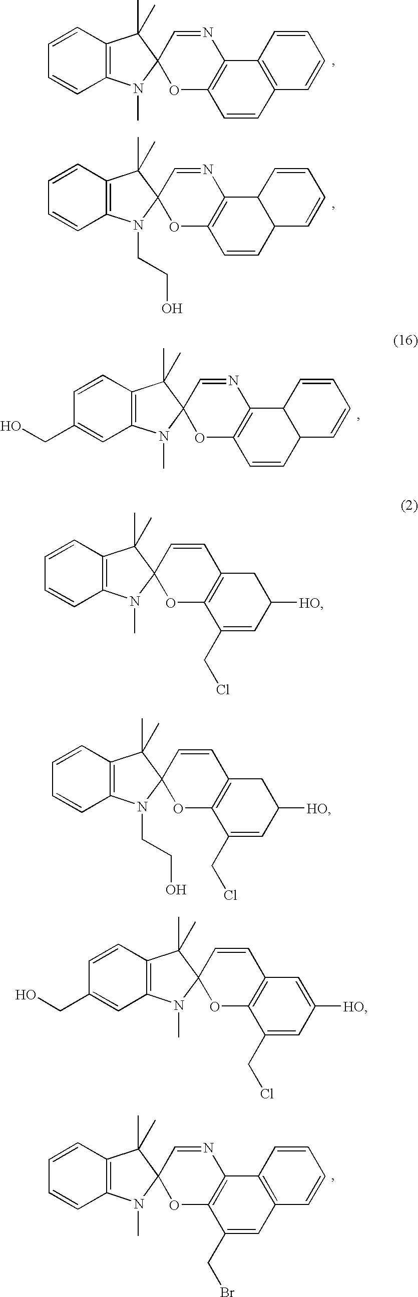

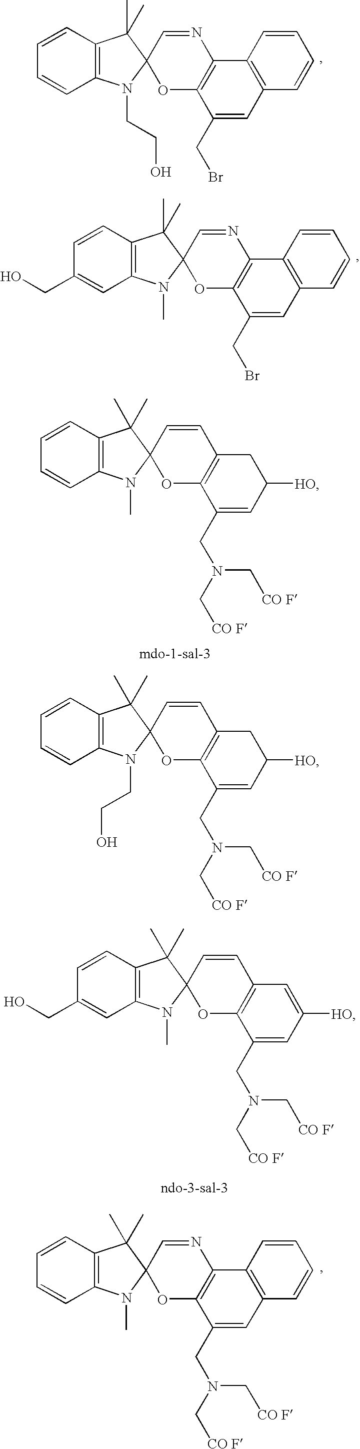

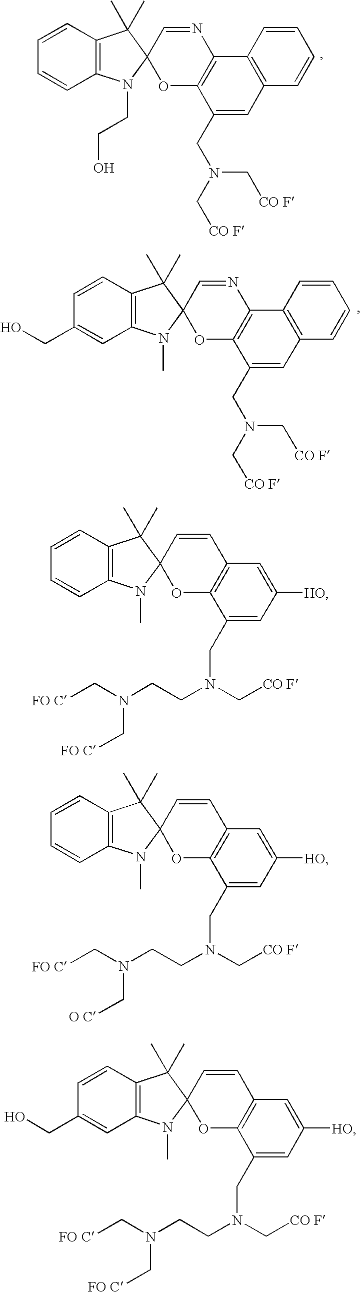

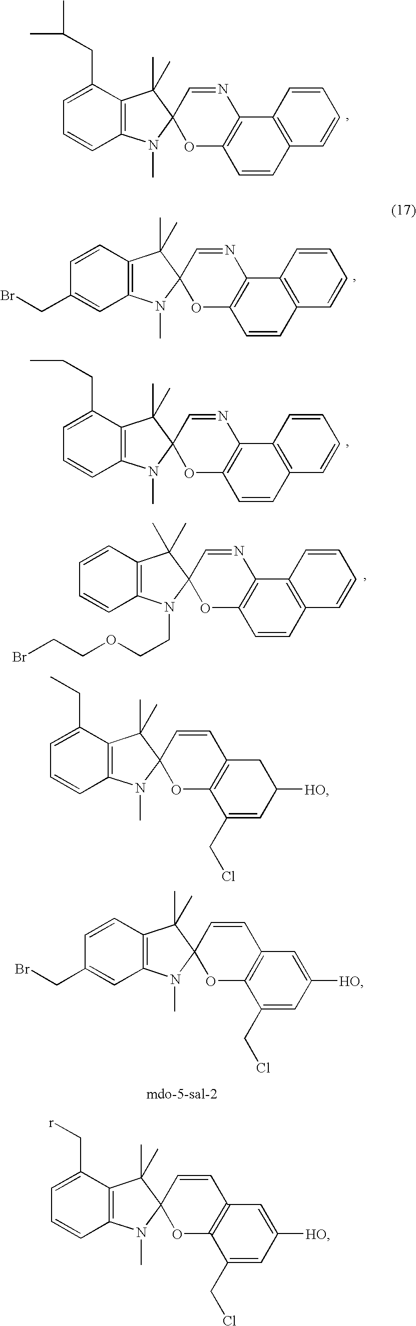

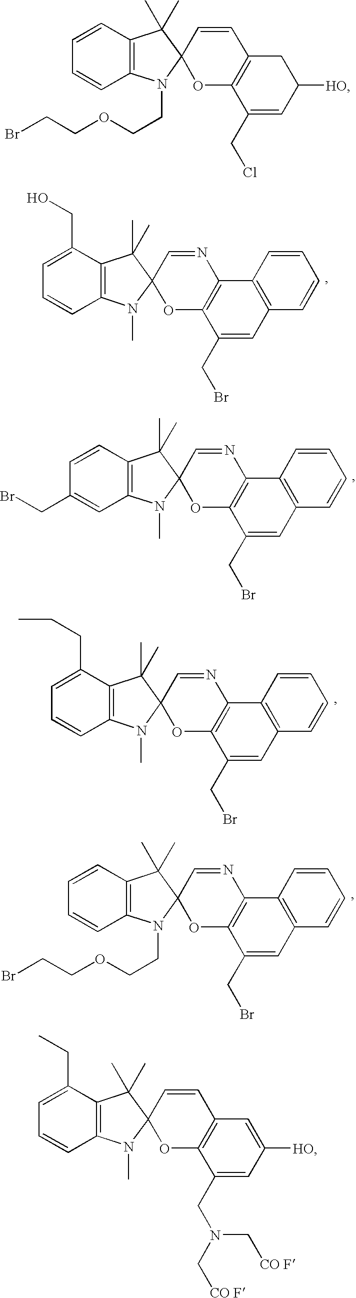

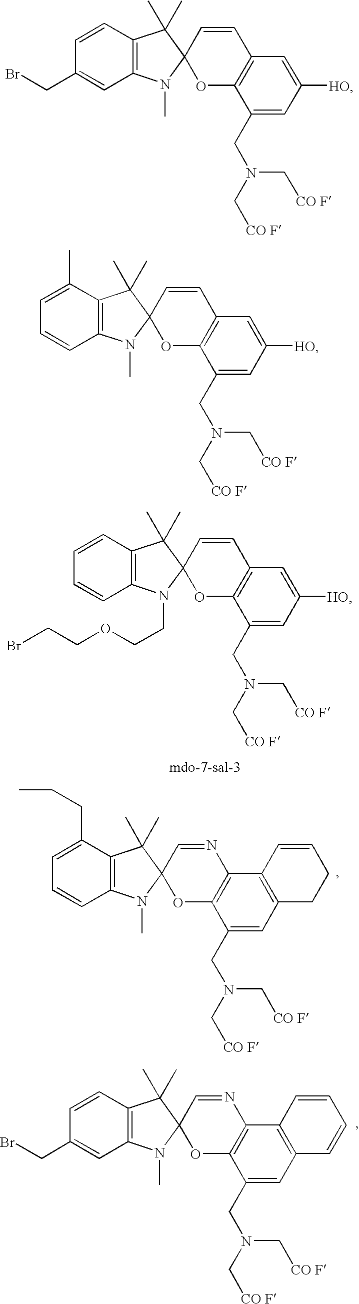

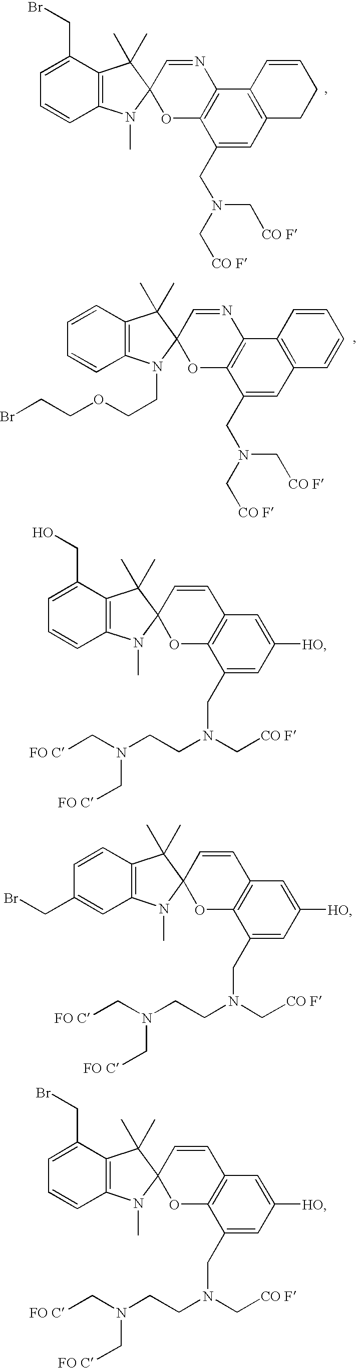

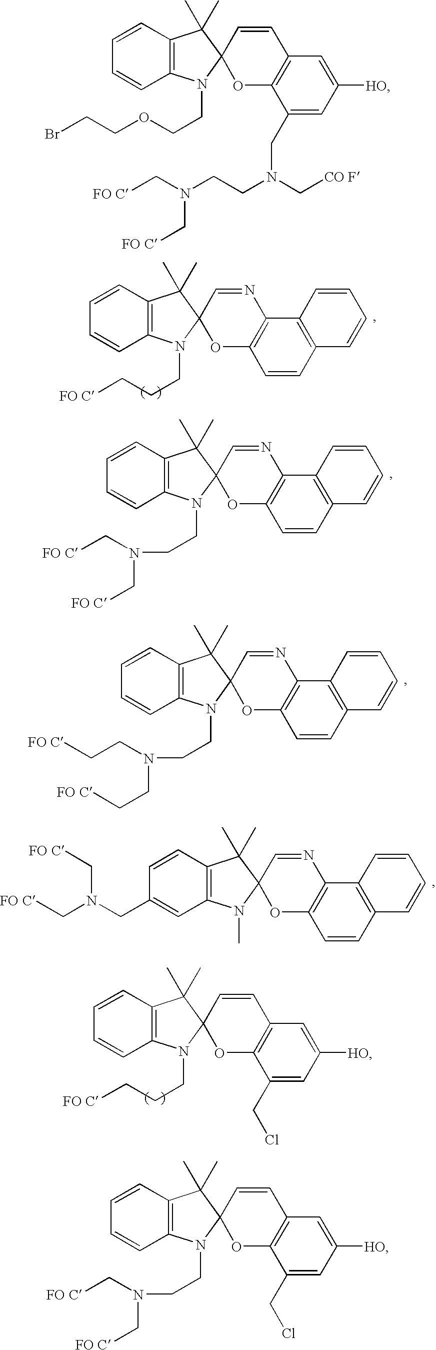

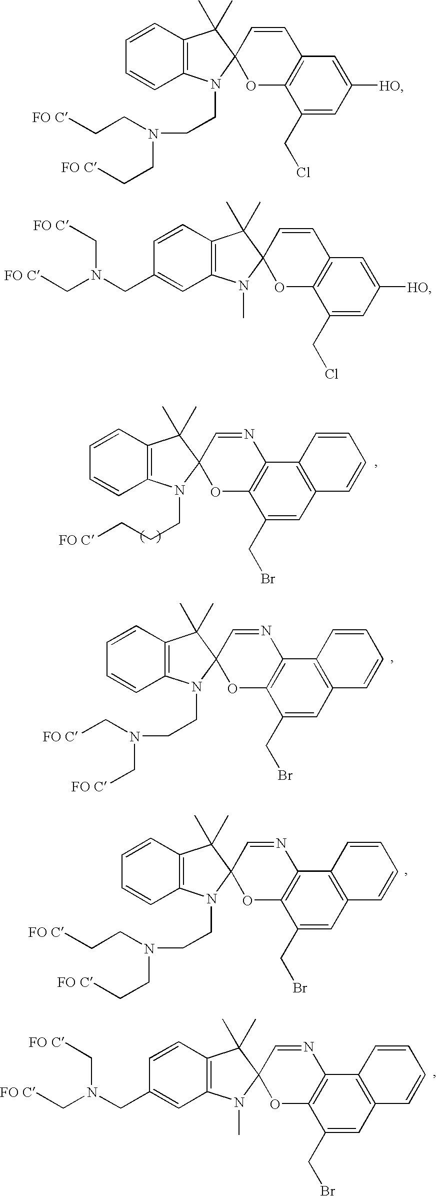

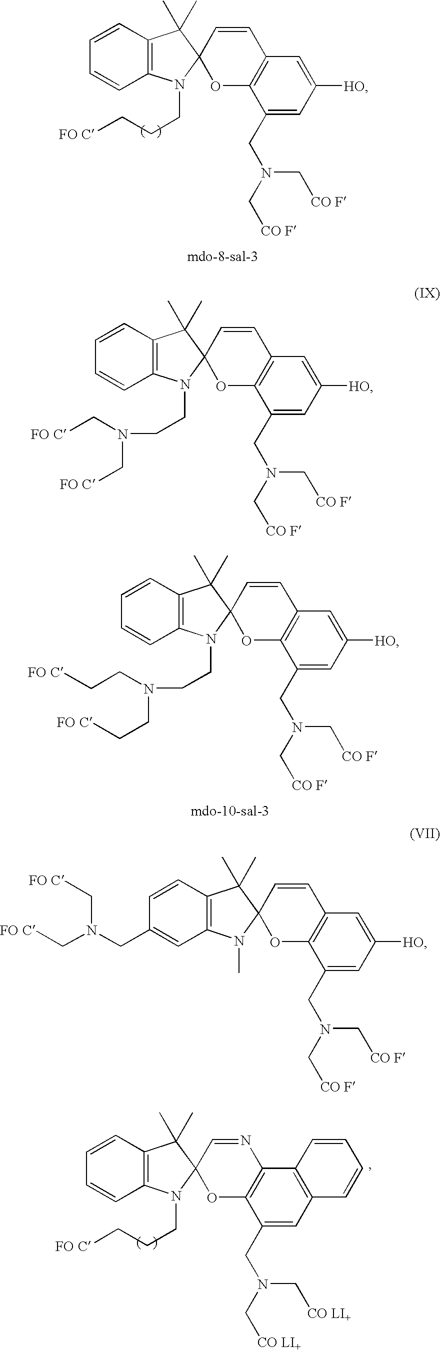

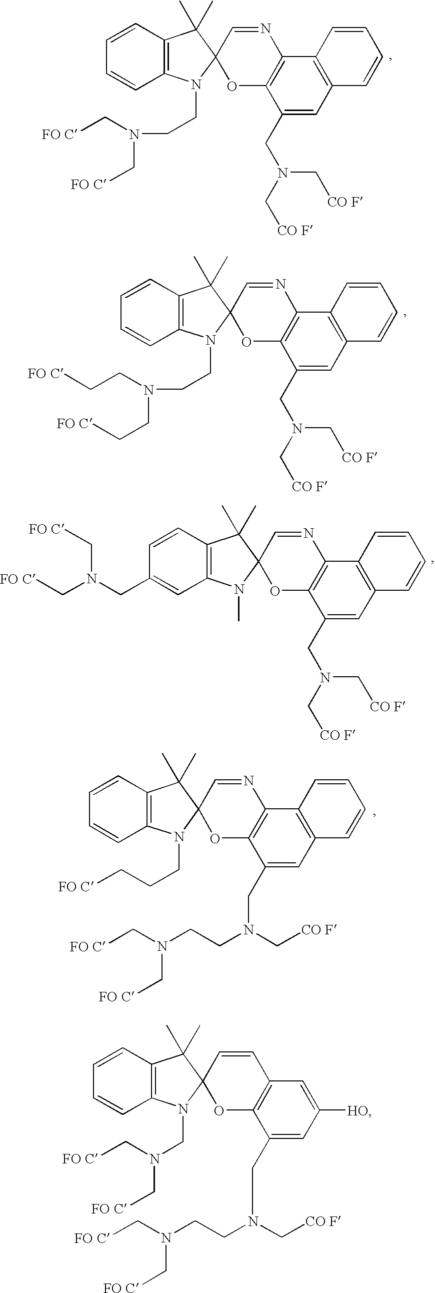

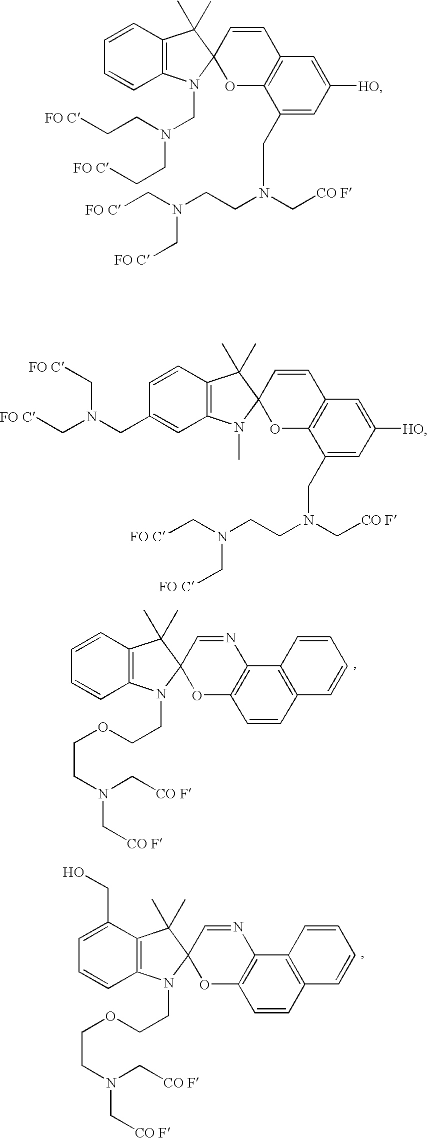

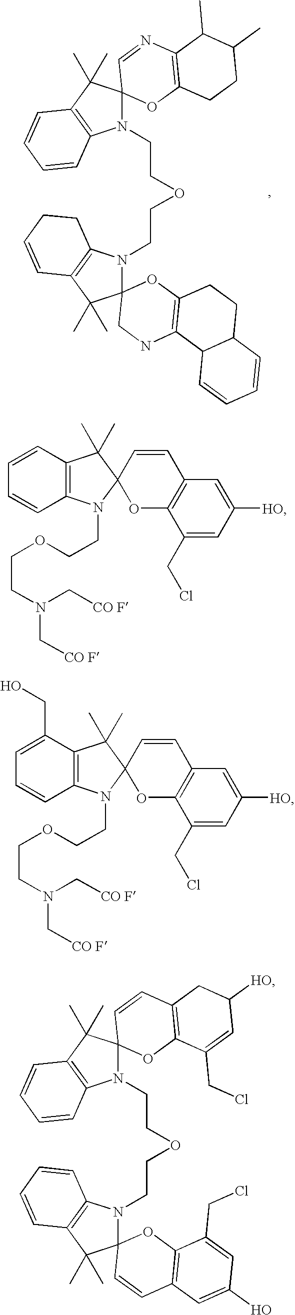

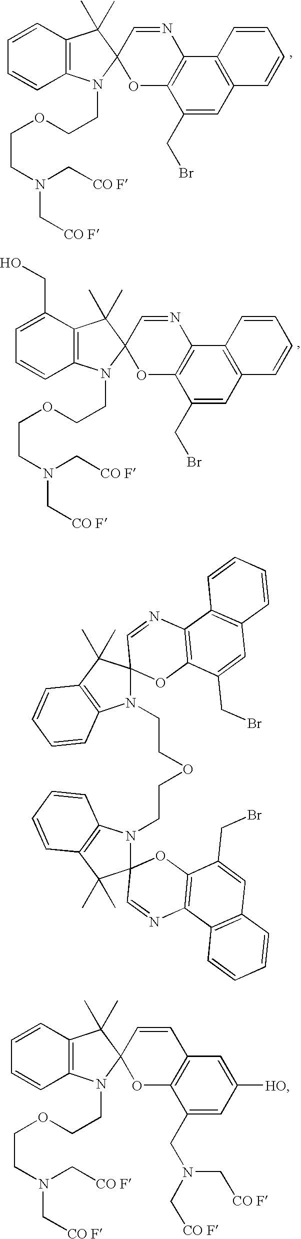

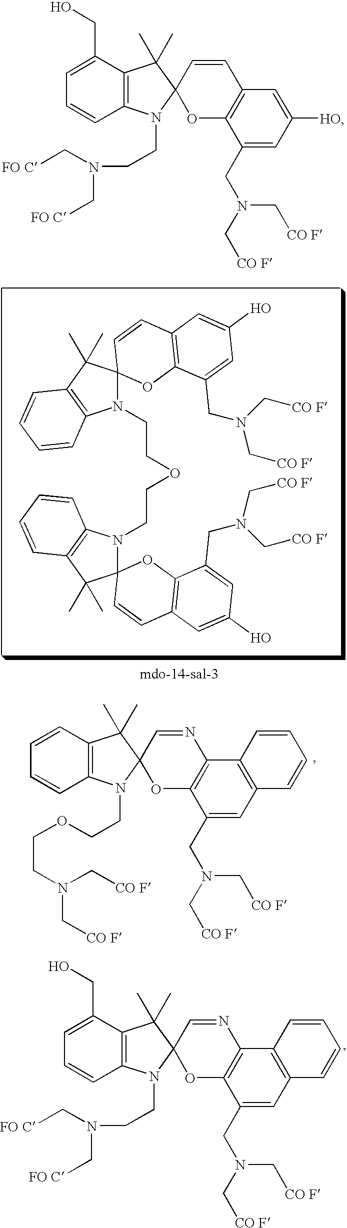

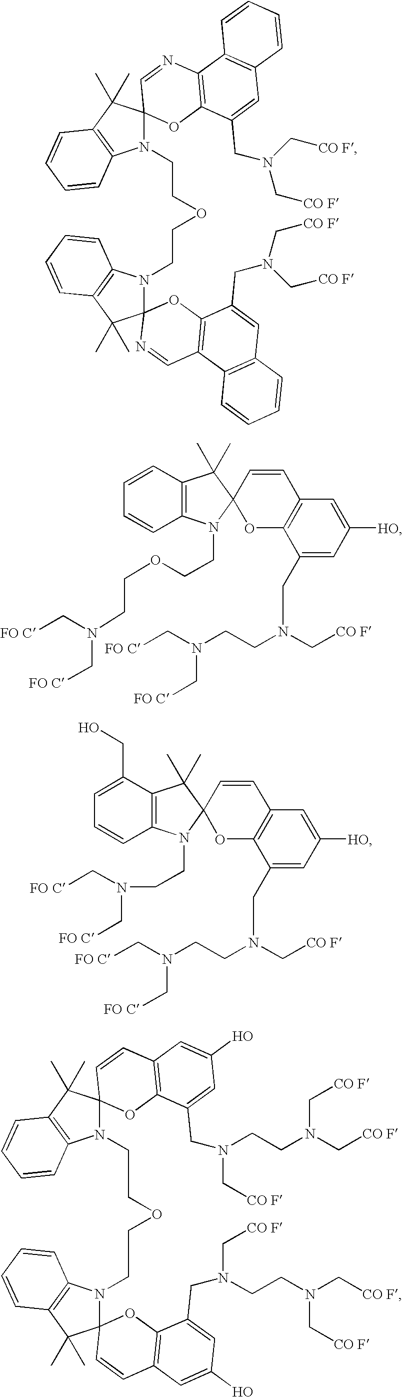

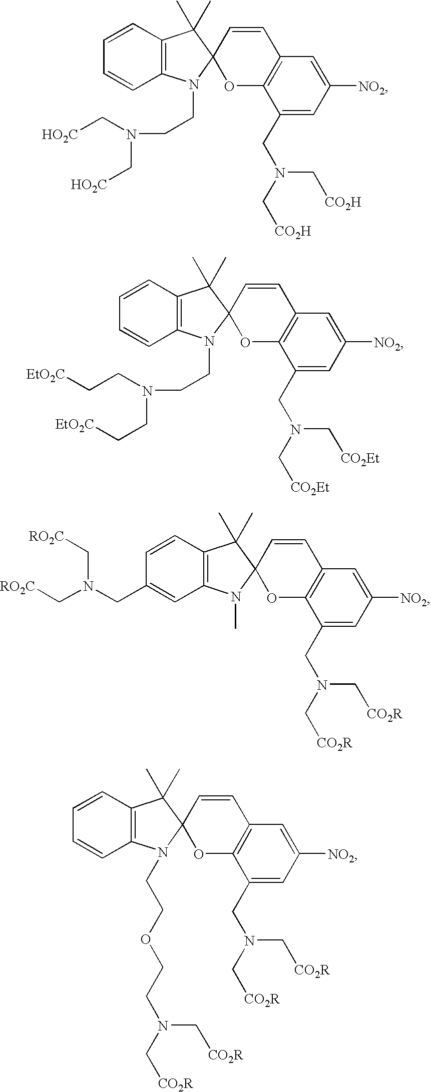

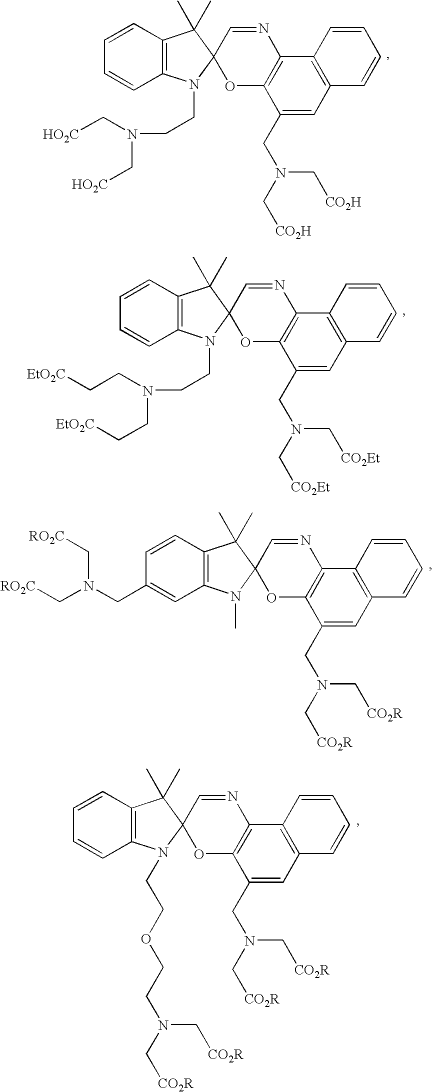

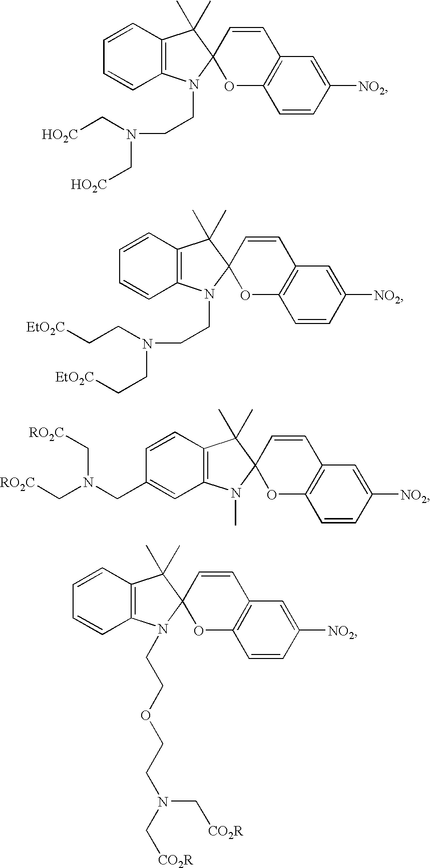

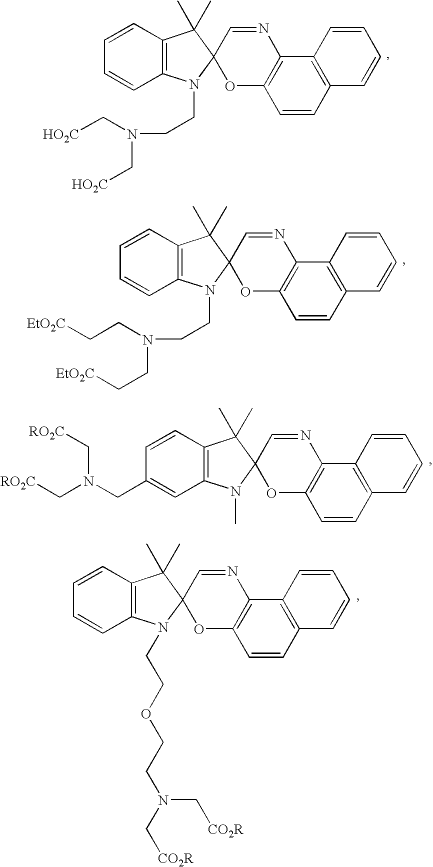

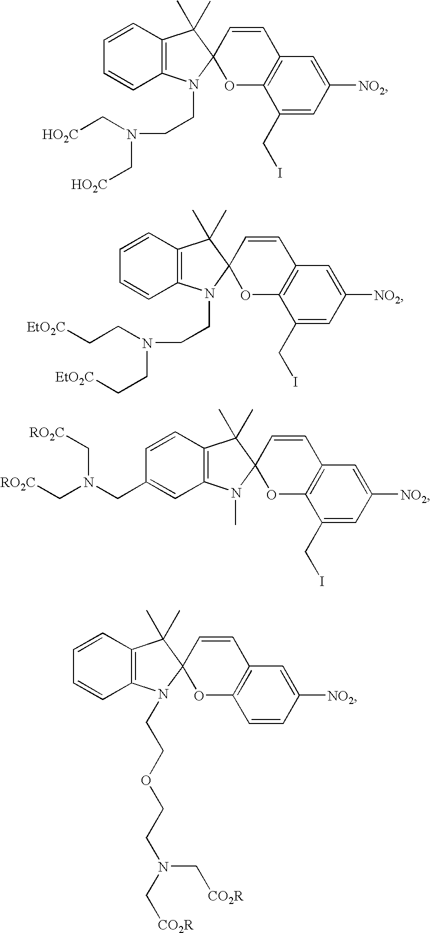

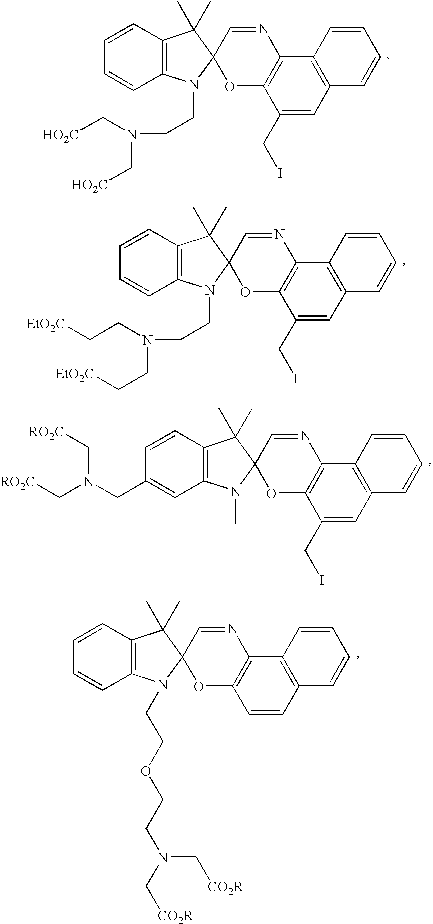

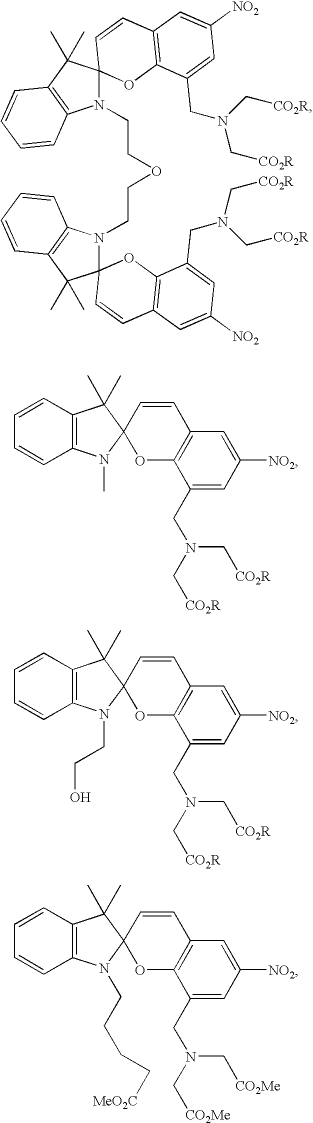

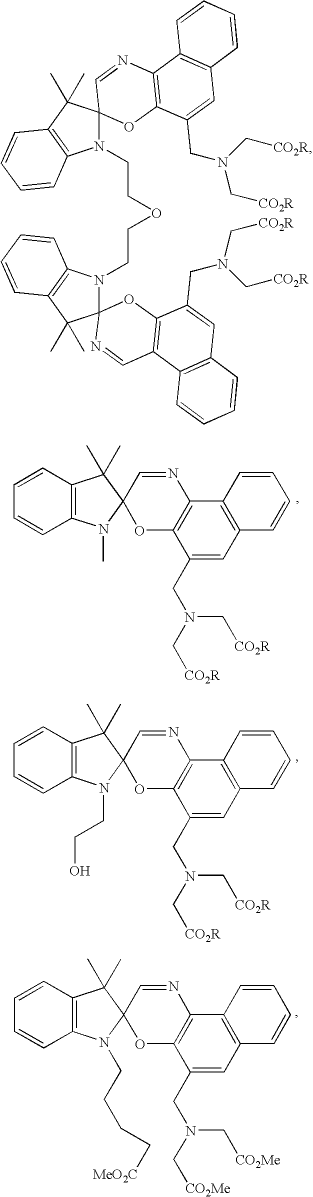

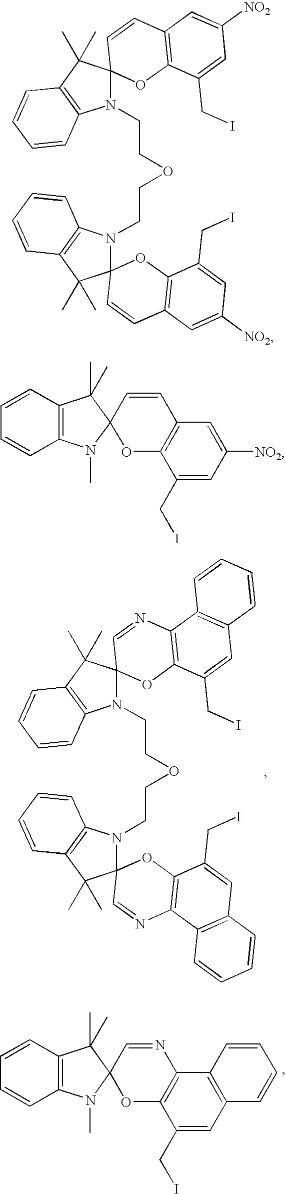

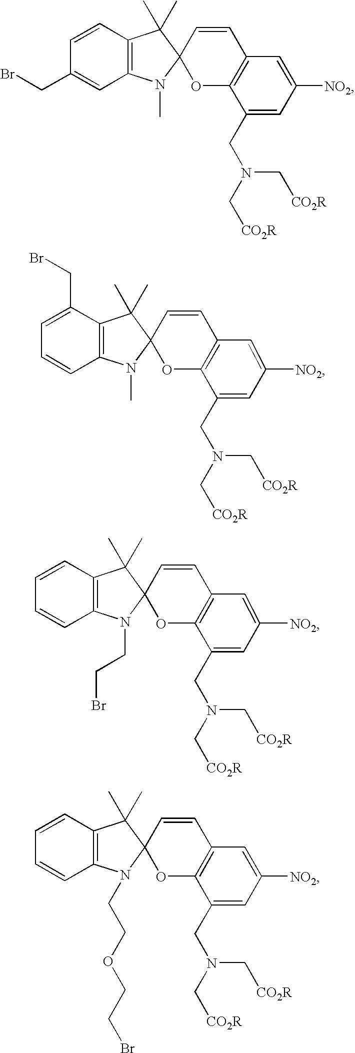

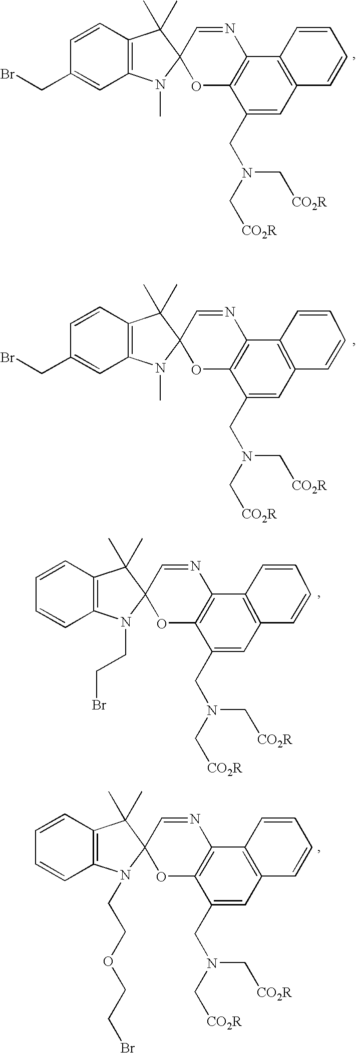

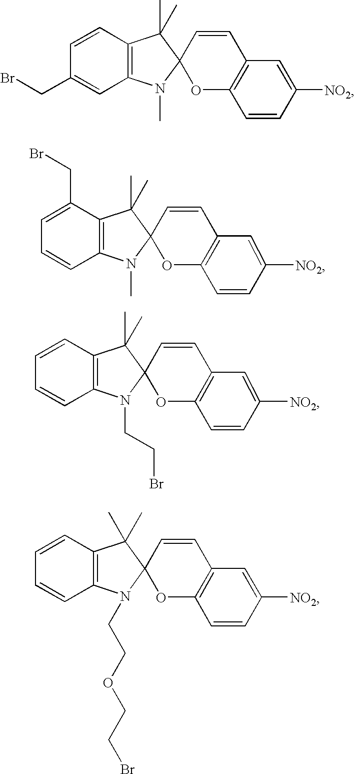

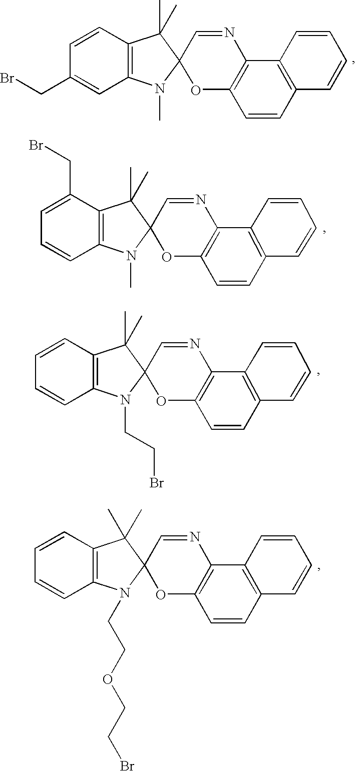

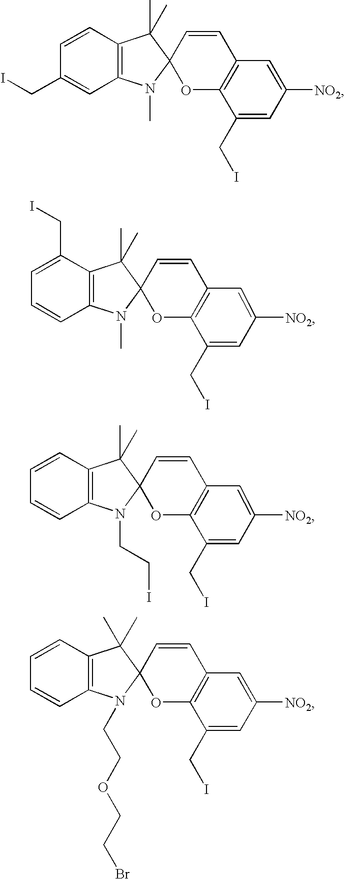

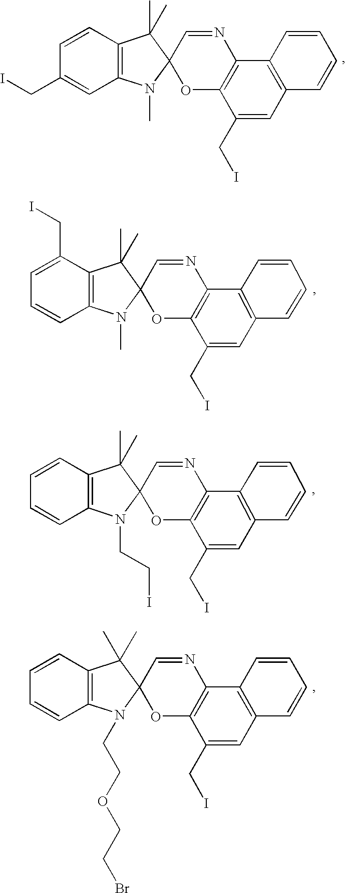

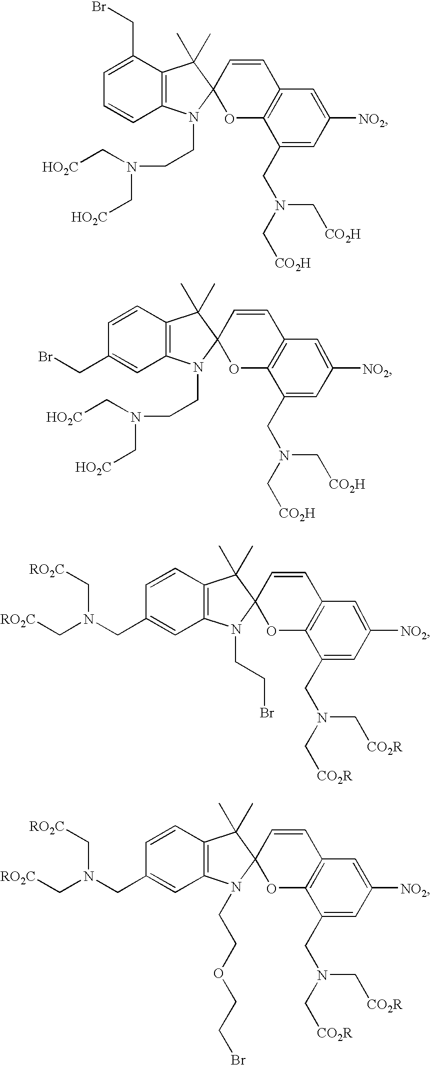

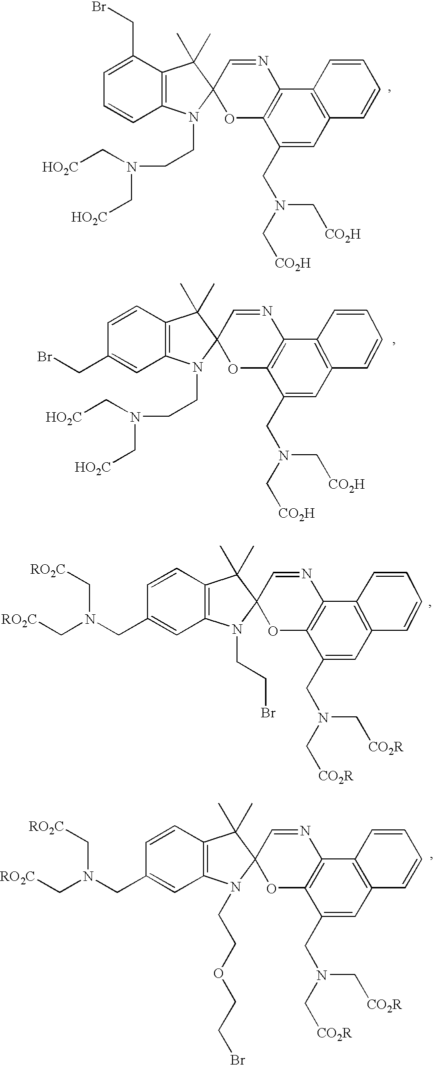

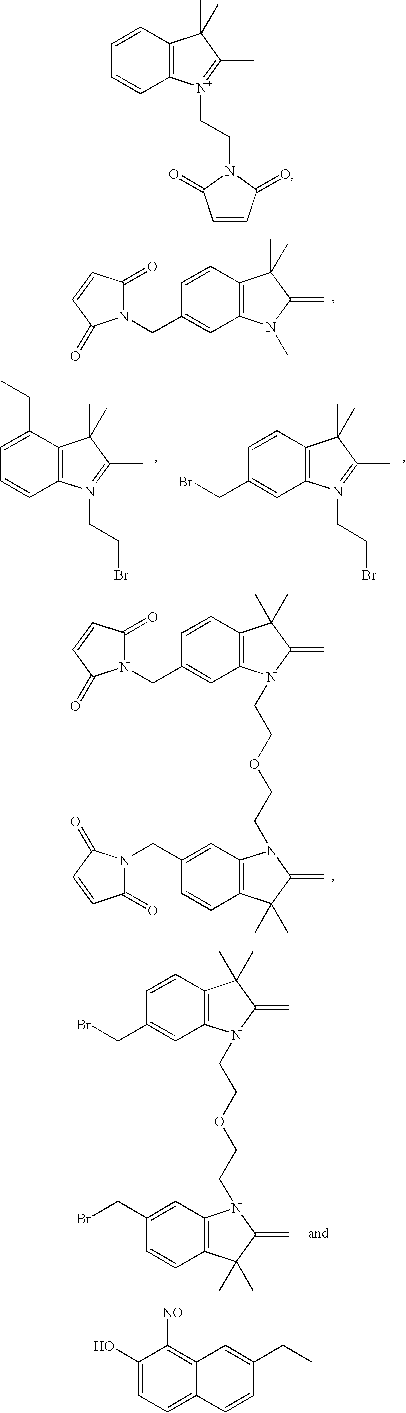

wherein R is independently selected from H, CH₃, C₂H₅ and C₃H₇.

The invention also provides a reversible optical photochromic probe comprising a compound or a derivative thereof as shown above. The probe is capable of undergoing light directed reversible transition between a first state and a second state. The first state is obtained by shining light of about 365 nm on the compound or derivative thereof, whereas the second state is obtained by shining light of about 545 nm to 620 nm on the compound or derivative thereof.

Another embodiment of the invention also provides a method of determining or controlling biomolecular interactions or activity. The method comprises the step of contacting said biomolecule with an optical photochromic probe of a compound or derivative thereof as shown above. Further, the biomolecular interactions may studied or determined using Foerster resonance energy transfer (FRET), fluorescence recovery after photobleaching (FRAP), photoactivation of fluorescence (PAF) technologies and Speckle microscopy. In this method, the optical photochromic probe is capable of undergoing light directed reversible transition between a first and second state. As discussed above, the first state is obtained by shining light of about 365 nm on the compound or derivative thereof and the second state is obtained by shining light of about 545 nm to 620 nm on the compound or derivative thereof. The biomolecules in this method include proteins, DNA, RNA, sugars, or ligands.

The present invention also provides a method of determining free or bound calcium or controlling calcium binding in a subject. The method comprises the step of contacting the subject with a reversible optical photochromic probe of a compound or a derivative thereof as shown above. The free or bound calcium determination or calcium binding is controlled by light directed reversible transition between a first state and a second state. Quantative calcium estimation and controlling calcium binding interactions may be determined using Foerster resonance energy transfer (FRET), fluorescence recovery after photobleaching (FRAP), photoactivation of fluorescence (PAF) technologies and Speckle microscopy. Further, the present invention also provides an optical photochromic probe which has at least two optical switches. Each optical switch may be independently controlled by light directed reversible transition between the first state and the second state.

The present invention also teaches a method of synthesizing a thiol reactive optical switch, comprising the steps of: (a) coupling an indoline derivative with a salycilaldehyde or nitrosonaphthol derivative to yield a spirobenzopyran or a spironaphthoxazine; and (b) conducting a halogen exchange reaction or bromination of alcohol or modified Mitsunobu reaction on the spirobenzopyran or spironaphthoxazine to yield a thiol reactive spirocompound useful as an optical switch. In this method, the indoline derivative is a compound selected from the group consisting of:

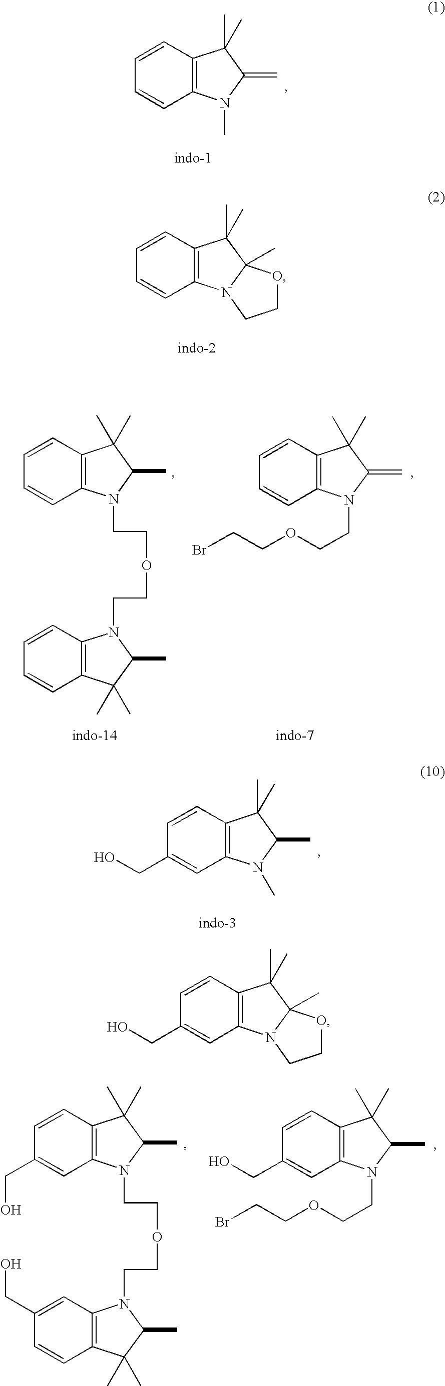

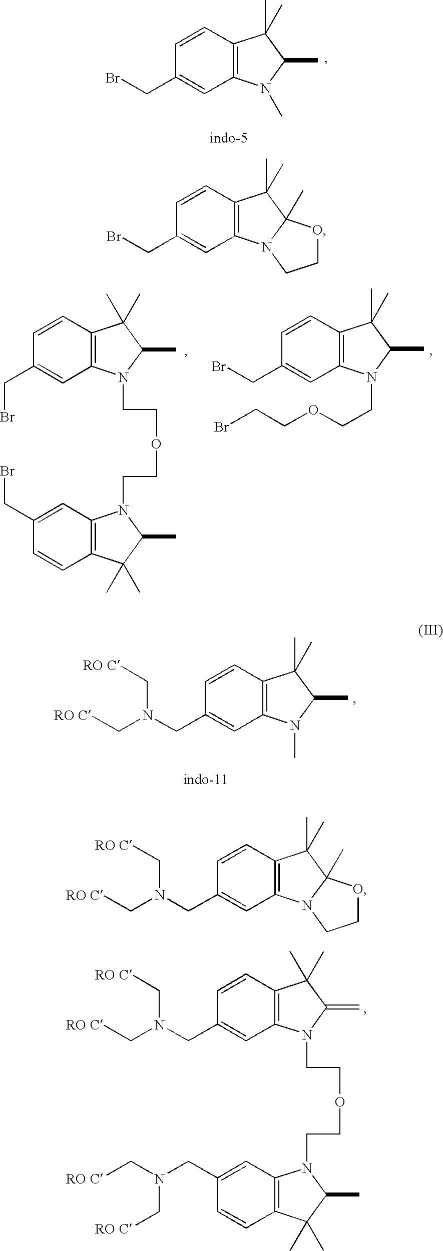

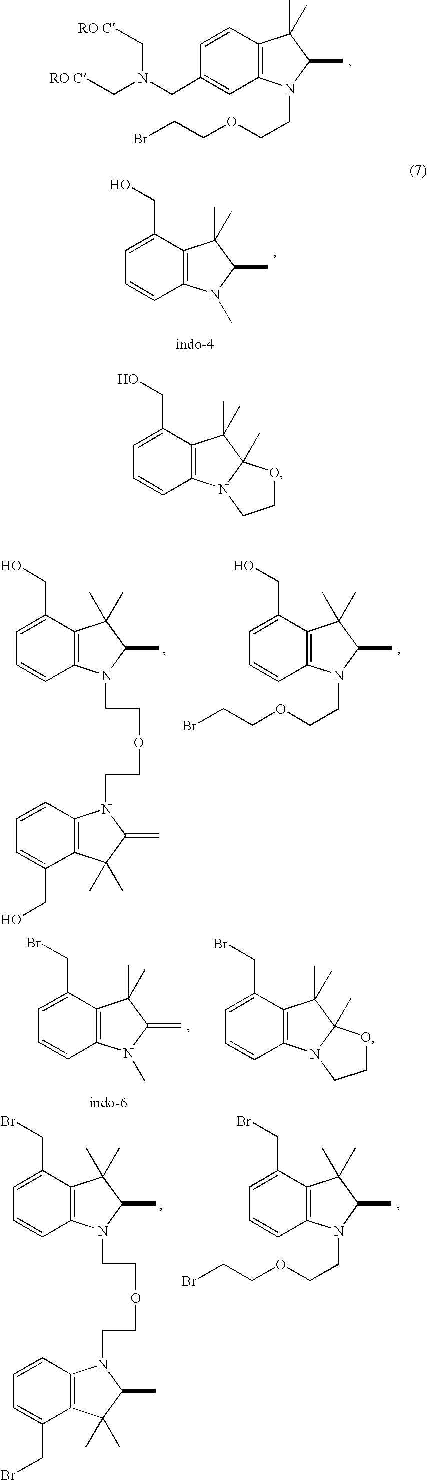

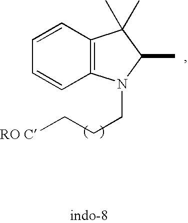

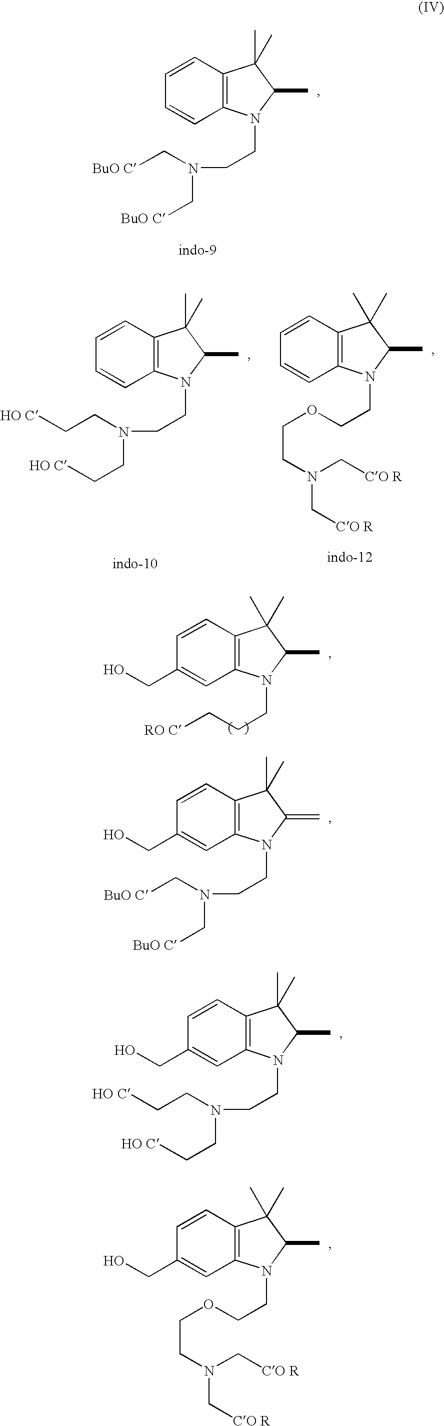

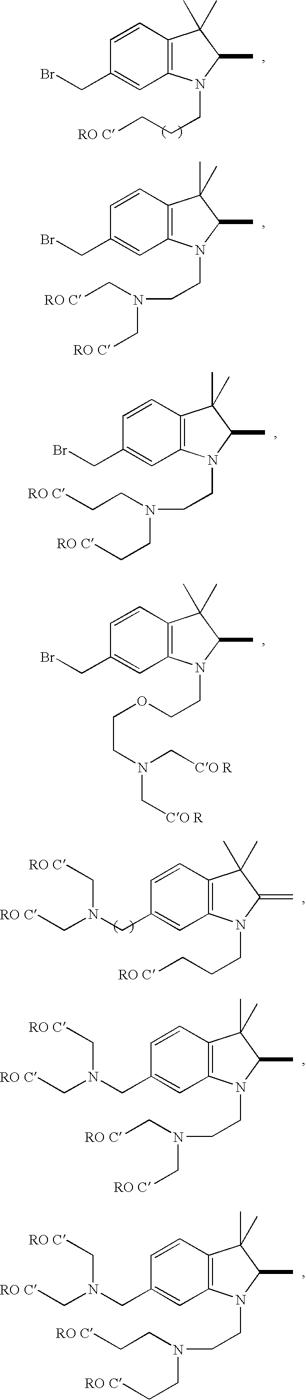

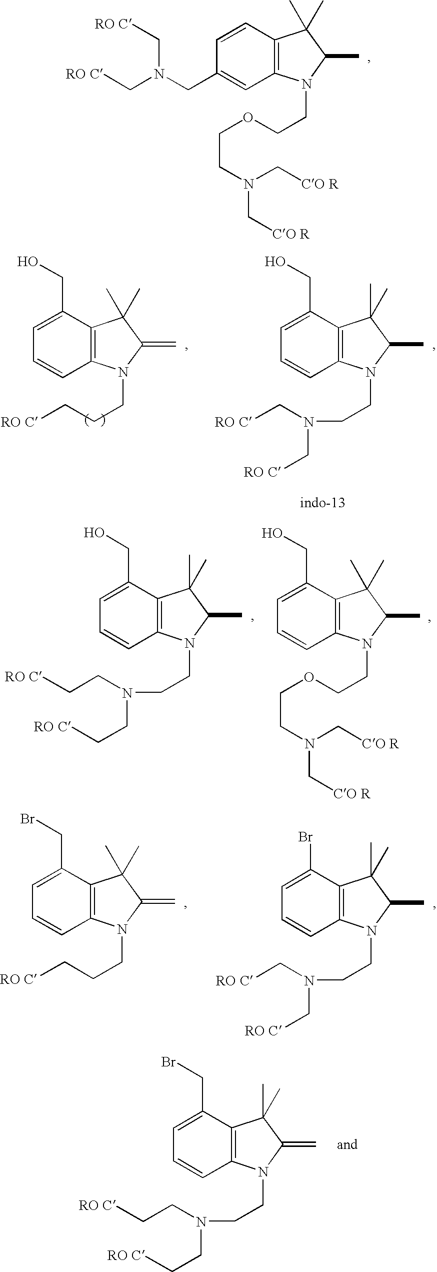

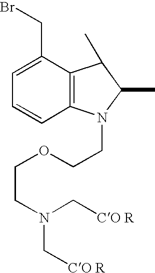

wherein R is independently selected from H, CH₃, C₂H₅ and C₃H₇.

Further, the spirobenzopyran or the spironaphthoxazine is a compound or a derivative thereof as shown above. The indoline derivative may be synthesized by a coupling reaction of an indole derivative and an alkyl halide.

In sum, the present invention represents new compounds and methods of using these compounds as photochromic probes. These and other objects and advantages of the present invention will become apparent from the detailed description and drawings accompanying the claims.

### DESCRIPTION OF DRAWINGS

FIG. 1. (A), Chemical structures of the thiol reactive, spirobenzopyrans described in this invention and the light-driven transitions between the SP state and the MC state. (B), Schematic representation of a new approach for reversible optical switching of functional interactions and activities of biomolecular conjugates of spirobenzopyran.

FIG. 2. (A), Absorption spectra of the MC state of the five thiol reactive probes described in this invention in ethanol at a concentration of 20 μM. The maximum absorption value of the lowest energy transition for each probe is normalized to a value of 1.0—the letters (a-e) refer to the compounds 3, 9, 1 2, 1 3 and 6 respectively. (B), Absorption spectra of the MC state of compound 1 2 in the following solvents :a), water; b), 1,2-propanediol; c), 2-proanol; d), acetonitrile; e), dichloromethane.

FIG. 3. (A), Technical fluorescence excitation spectrum of the G-actin conjugate of compound 12, showing the S₀—S₂ (centered at 370 nm) and S₀—S₁ (centered at 558 nm) excitation bands of MC. The emission was monitored at an emission wavelength of 650 nm. (B), Normalized, technical emission spectra of G-actin conjugates of compounds: a), 3 (624 nm); b), 12 (622 nm); c), 6 (628 nm); d), 13 (630 nm). The emission from the MC state in each conjugate was generated using an excitation wavelength of 370 nm.

FIG. 4. (A), Jablonski diagram to explain the observed absorption and fluorescence properties of the spirobenzopyrans studied in this invention. This diagram does not consider details of the photochemistry, which is believed to occur in the triplet state¹⁵. (B), Explanation of the anomalous polar solvent-induced blue-shift in the absorption of MC. Because the dipole moment of the MC excited singlet state is smaller than that for the ground state (14 D vs 20 D)²¹, the solvent has a minor influence on the energy of the MC excited singlet state, whereas the MC ground state is highly stabilized by specific dipolar MC-solvent interactions.

FIG. 5. Schematic representation of the linkage geometry between the thiol group on a biomolecule and the thiol reactive spirobenzopyran probes described in this study. To highlight the different geometries attainable using these probes the inventors use two points of reference for each probe—first, the sulfur atom on the biomolecule is fixed at the origin and second, the atom harboring the thiol reactive group on the spirobenzopyran is forced to assume a position on the x-axis. The SP and MC states of the five chromophores have the same stereochemical arrangement.

FIG. 6. (A), Optical switching between the SP and MC states on G-actin. Absorption spectra of compound 13 attached to cysteine-374 on G-actin in response to sequential irradiation with UV and visible light. The letters refer to: a), Pre-irradiated SP state; b), 30 seconds illumination of the conjugate with 365 nm light; c), 30 seconds illumination of the MC conjugate [spectrum (b)] with 546 nm light; d), 30 seconds illumination of the SP conjugate [spectrum (c)] with 365 nm light. (B), Normalized absorption spectra for the lowest energy transition of the MC state of the five thiol reactive probes attached to cysteine-374 on actin. The letters (a-e) refer to the compounds 9, 13, 3, 6 and 12 respectively. (C), Normalized absorption spectra for the lowest energy transition of the MC state of the five thiol reactive probes attached to a cysteine residue in BSA. The letters (a-d) refer to the compounds 13, 9, 12 and 6 respectively.

FIG. 7. (A), Time dependent absorption spectra of the lowest energy transition of the MC state of compound 12 dissolved in ethanol at a concentration of 20 μM at 20° in the dark—the spectra, from top to bottom, were recorded at the following times: a) 0; b), 60 s, c), 120 s; d), 180 s; e), 240 s; f), 360 s; g), 420 s; h), 480 s. The lowermost curve is the absorption spectrum of the non-irradiated SP state. (B), The rate for the thermally driven MC to SP transition was determined by analyzing the log of the maximum absorption value for each spectrum shown in FIG. 7A as a function of time. The reaction rate of 270 sec⁻¹ was calculated using a least squares fitting procedure. (C), Absorption spectra of the MC-G-actin conjugate (compound 9) as a function of time in the dark. Spectra are as follows: a), before irradiation; b), after 30 second irradiation with 365 nm light; c), after 5 minutes in the dark at 20°; d), after 22 minutes in the dark at 20°; e) after irradiation with 546 nm light for 30 seconds

FIG. 8. Schematic for optical switching of calcium—both chelate shown below have been synthesized as the membrane permeable esters (ethyl and t-buyl) and as the free acid. Compound VIII is designed to release Ca²⁺ following formation of MC with 365 nm light and to chelate Ca²⁺ in SP state achieved by 546 nm irradiation of MC. Compound X on the other hand is designed with its more flexible linker groups to chelate Ca²⁺ in MC state and to release Ca²⁺ following irradiating the MC state with 526 nm light.

FIG. 9. Schematic representation of general synthesis of spiro compounds, namely spirobenzopyran and spironaphthoxazine.

FIG. 10. Combinatorial table of indolines and salicylaldehydes.

FIG. 11. Combinatorial table of spiro compounds for indoline derivatives indo-1 to indo-7.

FIG. 12. Combinatorial table of spiro compounds for indoline derivatives indo-8 to indo-14.

FIG. 13. Fluorescence emission from the MC state.

FIGS. 14. (A) and (B) Combinatorial table of calcium probes.

FIG. 15. Irradiation of SP calcium switch for compound X loaded into Hela cells with 365 nm light for 0.5 seconds.

FIG. 16: A), Normalized absorption spectra for the lowest energy transition of the MC state of the five thiol reactive probes attached to a single cysteine residue in BSA. The letters (a-e) refer to the compounds 13 (a), 9(b), 12(c), 6 (d) and 3 (e) respectively; B), Absorption spectra of the spironaphthoxazine (17) labeled G-actin (Cys-374) in the SP-state (a), and MC state (b). The MC state was generated by irradiating a 50% glycerol solution of (a) with 365 for 10 seconds.

FIGS. 17. (A), (B), (C) and (D) depict synthesis scheme for calcium probes and intermediates.

FIG. 18. Regulation of protrusion A), Resting cells—low Ca²⁺. Actin filament barbed-ends are capped by CP and Gelsolin. B), A local increase in Ca²⁺. Gelsolin severs filaments and, along with CapG and CP caps their barbed-ends. C), A local increase in PIP₂ triggered by Rac1 activation of PI5-kinase uncaps barbed-end.

FIG. 19: A), Generalized structure of the fluorescent KabC derivatives. Confocal images of fluorescein-KabC (B) and TMR-KabC (C) in live 3T3 cells showing staining of actin filaments at sites of membrane protrusion (B) and the actin cortex (C). KabC probes do not stain stress fibers to any significant degree.

FIG. 20: Caging of CapG (A): Absorption spectra of 120 L samples of CapG (2), caged CapG (curve 1). The labeling ratio of NVOC/CapG is approximately 4/1; (B): Fluorescence spectra of Prodan-G-actin (1), Prodan-G-actin +10 μM CapG (2), and Prodan-G-actin +10 μM caged CapG (3); (C) CapG binds to Prodan-G-actin and inhibits polymerization (3). Caged CapG shows almost normal polymerization kinetics (2) compared to the control Prodan-actin (1).

FIG. 21: (A), Depolymerization kinetics of Prodan-F-actin diluted to 100 nM in the absence (a, red) or presence of 1 μM cofilin (b), 1 μM caged cofilin (c, green) and 1 μM uncaged cofilin (d); (B), Light-directed activation of caged constitutively active cofilin inhibits cytokinesis in fertilized eggs.

FIG. 22: Properties of MC-fluorescence. (A), Emission spectra of MC-G-actin conjugate using alternate cycles of 365 nm and 546 nm excitation; (a) 1^{st} SP; (b), 1^{st} MC; (c), 2^{nd} SP; (d), 2^{nd} MC state. (B), Montage of video frames demonstrating optical switching of MC-fluorescence in the proposed Ca²⁺-optical switch (VIII) within NIH 3T3 cells. Excitation wavelength 546 nm, emission >590 nm; Frame 1, prior to 0.5 sec 365 nm excitation of cells; Frames 3, 4 and 5: 6.50,13.33 and 16.33 seconds after 1 st 365 nm pulse (Image #2); Frames 6, 7 and 8: 5.87 and 11.67 seconds after 2^{nd} 365 nm pulse (Image #6); Frames 10 and 11: 2.50 and 5.80 seconds after 2^{nd} 365 nm pulse (image #9); Frame 13: 6.0 seconds after 3^{rd}, 365 nm pulse (Image # 12); Frame 14: Phase image of the NIH 3T3 cells.

FIG. 23: Scematic for regulation of cell protrusion: (2): Regulation of barbed ends by CapG activated by: (a), caged CapG; (b), a CapG optical switch; (3): Capping of barbed ends by Ca²⁺ transients triggered by (a), receptor activation; (c), caged DM-Nitrophen/caged NP-EDTA; (c), optical switch Ca²⁺ chelators; (4): Uncapping of barbed ends by PIP₂ transients triggered by: (a), receptor activation (b), light-directed activation of caged Rac1 and (c), light directed activation of caged PIP₂; (d), light directed activation of caged cofilin; (5): Dissecting and mapping the receptor activated signaling pathway leading to cell protrusion

Although the invention is amenable to various modifications and alternative forms, specifics thereof has been shown by way of examples in the drawings and will be described in detail. It should be understood, however, that the invention is not limited to the particular embodiments described. On the contrary, the invention is to cover all modifications, equivalents, and alternatives falling within the scope of the scope and spirit of the present invention.

### DETAILED DESCRIPTION

General

Before the present methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

As defined herein, the term “isomer” includes, but is not limited to strereoisomers and analogs, structural isomers and analogs, conformational isomers and analogs, positional isomers and analogs and the like. In one embodiment, this invention encompasses the use of different positional isomers of a photochromic compounds as described in this invention. It will be appreciated by those skilled in the art that the photochromic compounds useful in the present invention may contain a chiral center. Accordingly, the compounds used in the methods of the present invention may exist in, and be isolated in, optically-active or racemic forms. Some compounds may also exhibit polymorphism. It is to be understood that the present invention encompasses the use of any racemic, optically-active, polymorphic, or stereroisomeric form, or mixtures thereof, which form possesses properties useful in obtaining reversible photochromic probes as described and claimed herein.

This invention further includes method utilizing derivatives of the photochromic compounds. The term “derivatives” includes but is not limited to ether derivatives, acid derivatives, amide derivatives, ester derivatives and the like. In addition, this invention further includes methods utilizing hydrates of the photochromic compounds. The term “hydrate” includes but is not limited to hemihydrate, monohydrate, dihydrate, trihydrate and the like.

As defined herein, “contacting” means that the photochromic compound used in the present invention is introduced into a sample containing the receptor in a test tube, flask, tissue culture, chip, array, plate, microplate, capillary, or the like, and incubated at a temperature and time sufficient to permit binding of the photochromic compound to a receptor. Methods for contacting the samples with the photochromic compound or other specific binding components are known to those skilled in the art and may be selected depending on the type of assay protocol to be run. Incubation methods are also standard and are known to those skilled in the art.

In another embodiment, the term “contacting” means that the photochromic compound used in the present invention is introduced into a subject, and the compound is allowed to come in contact in vitro, i.e. in a test tube, or in vivo, i.e. in cells or tissues of living organisms, for example, humans. In certain embodiments, the present invention encompasses contacting the compounds useful in the present invention to a patient or subject.

The Invention:

The present invention generally provides compounds and methods for using reversible photochromic compounds as probes. In one embodiment, the present invention provides a compound or a derivative selected from the group consisting of:

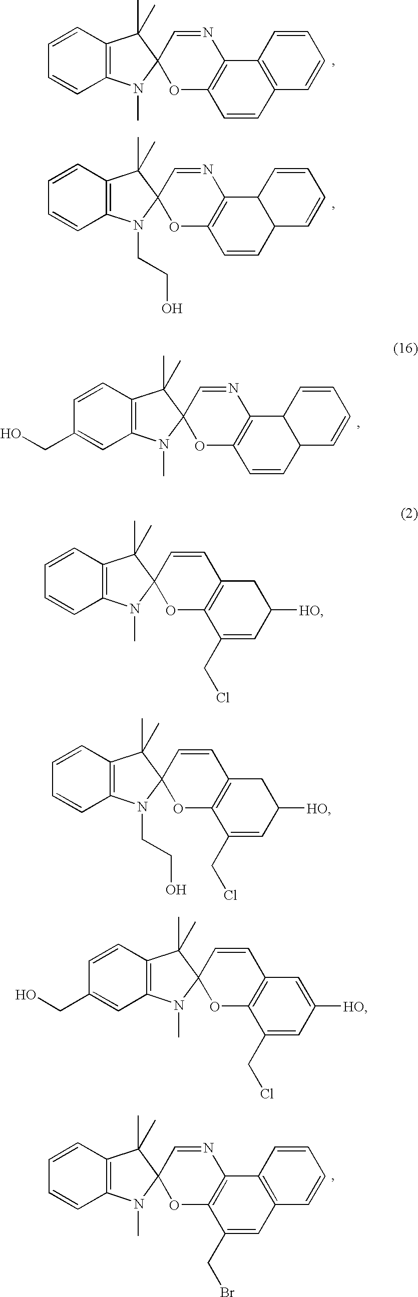

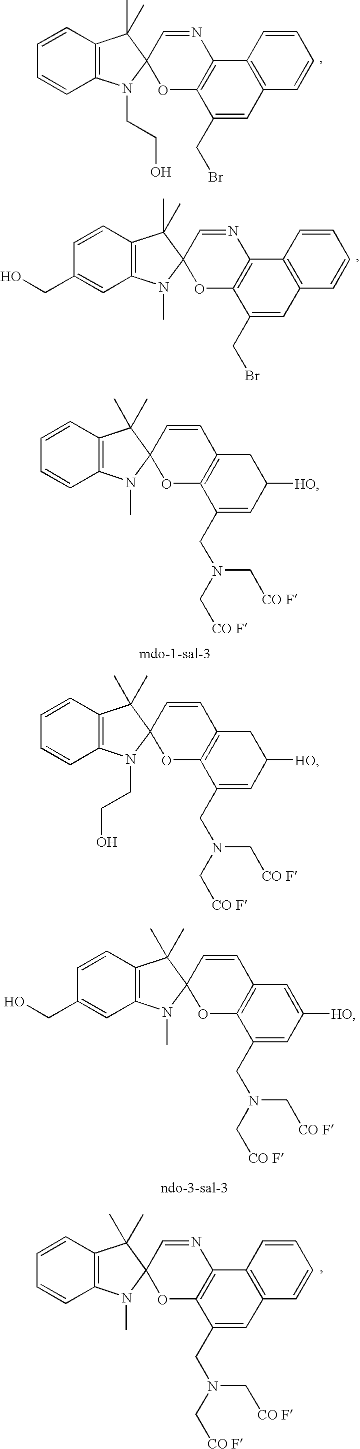

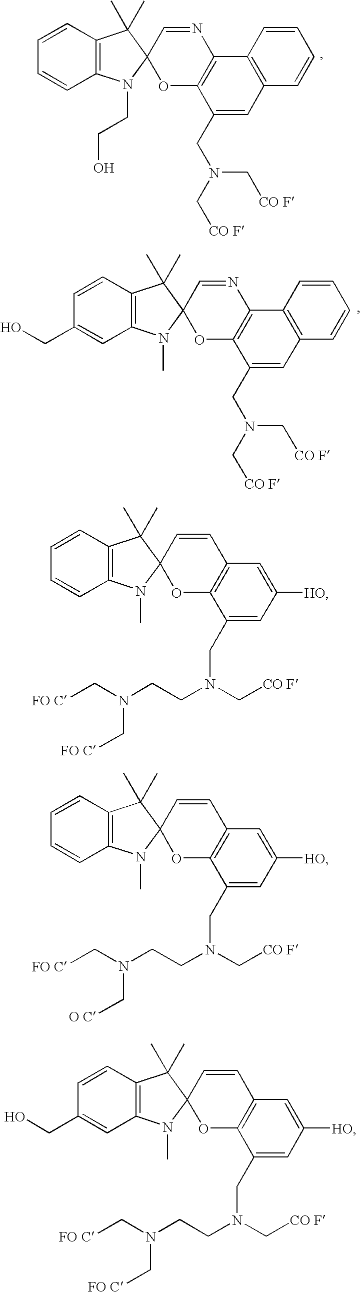

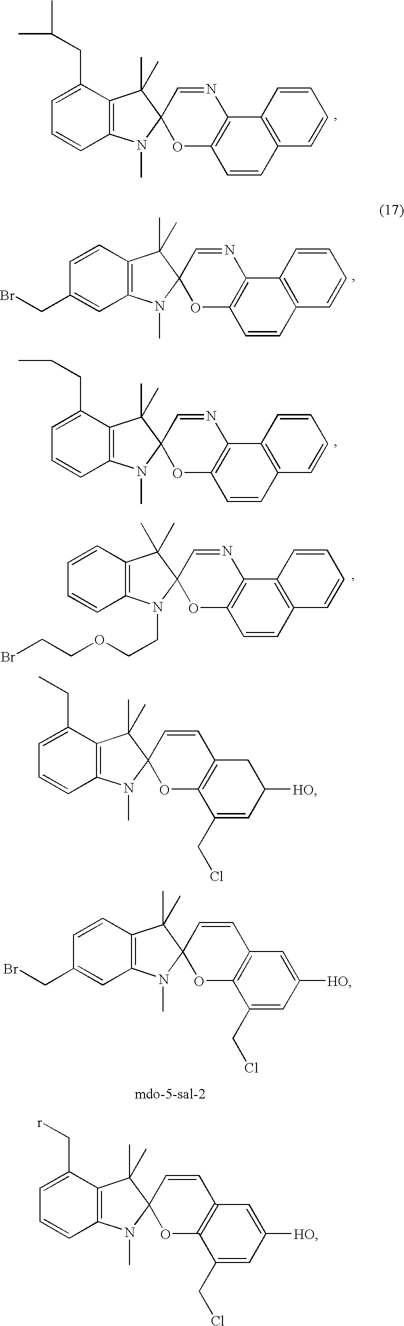

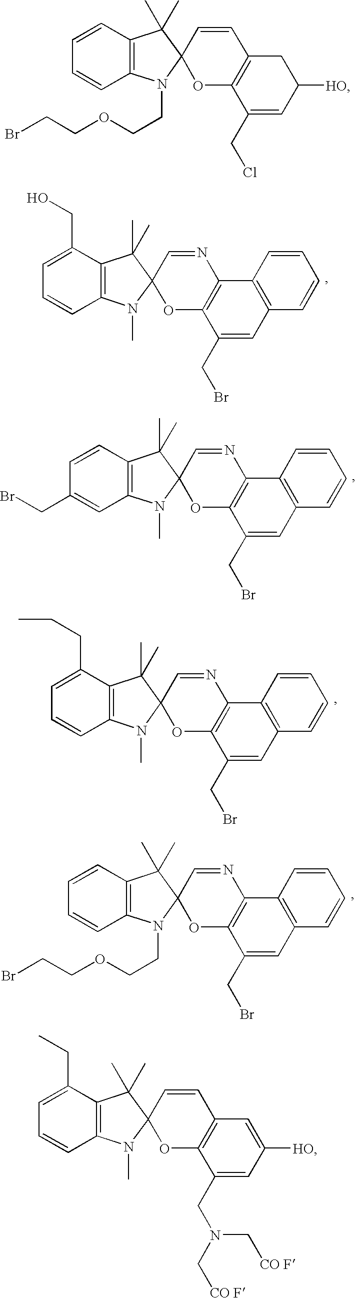

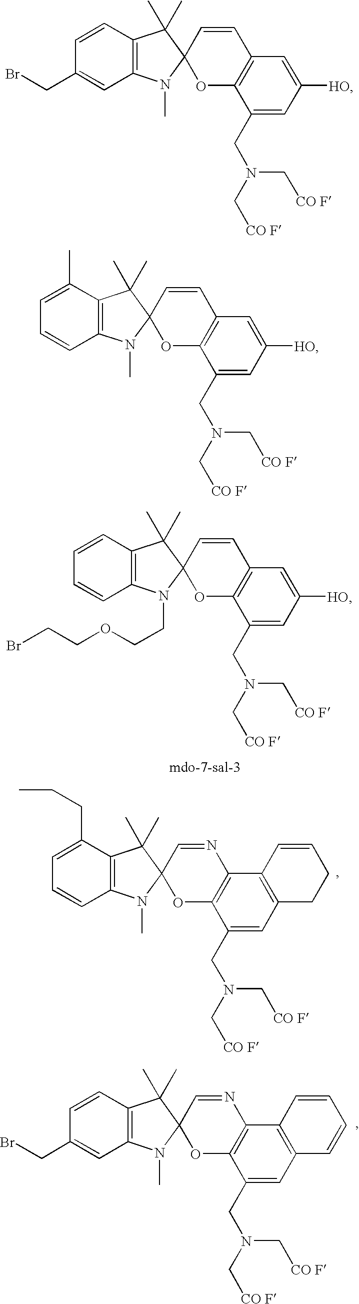

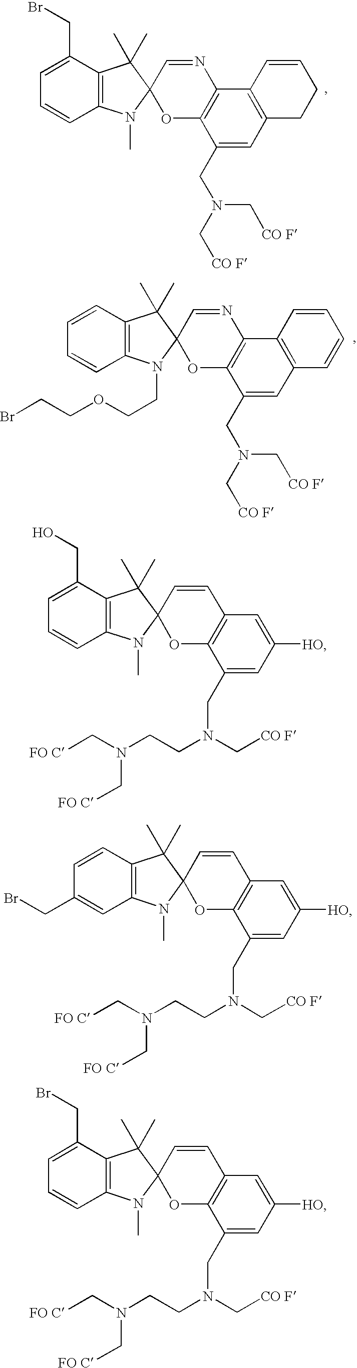

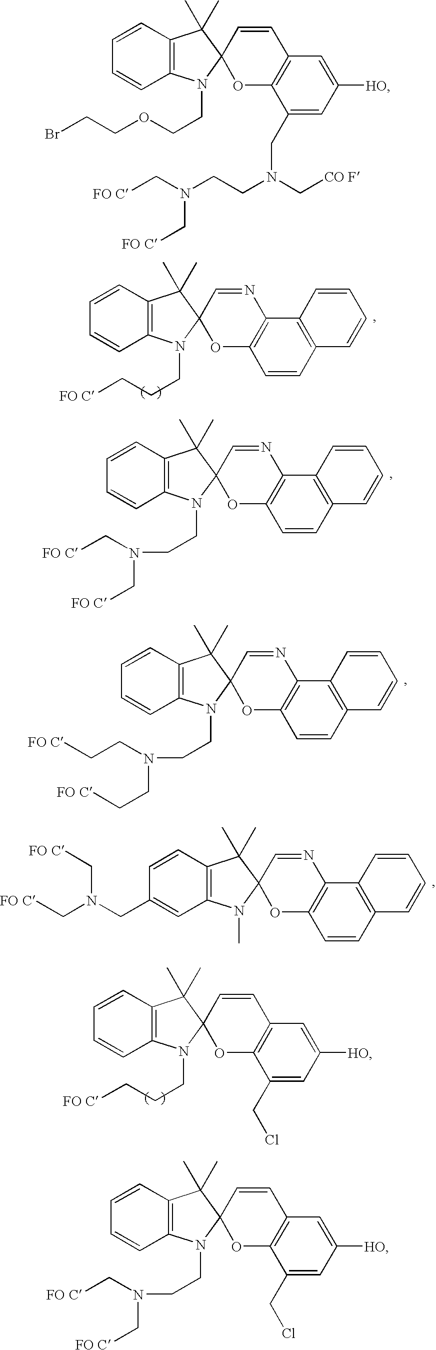

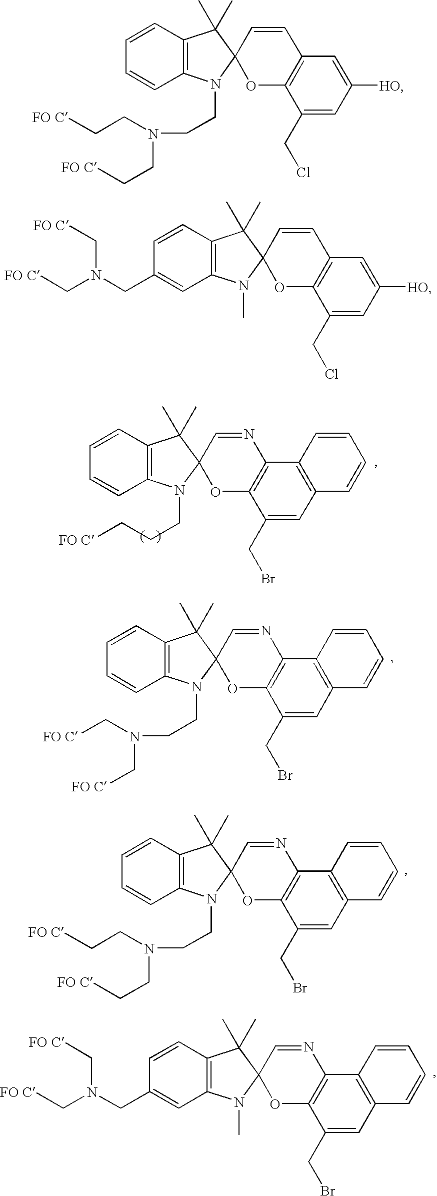

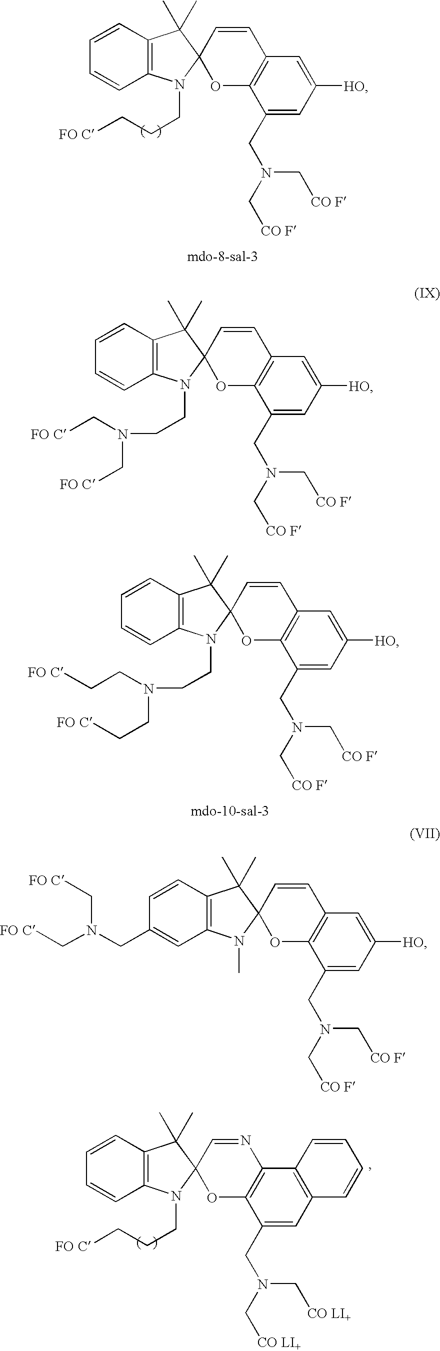

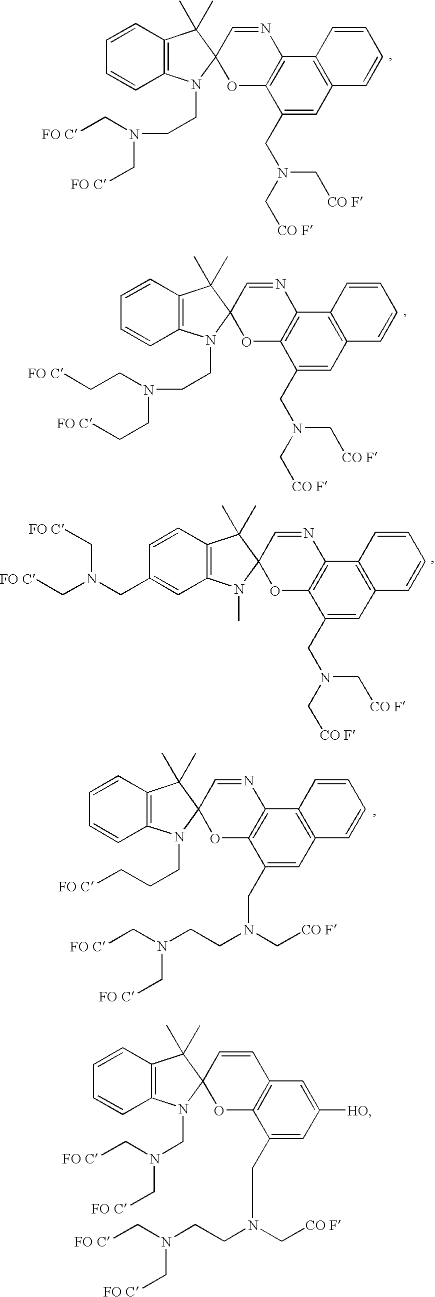

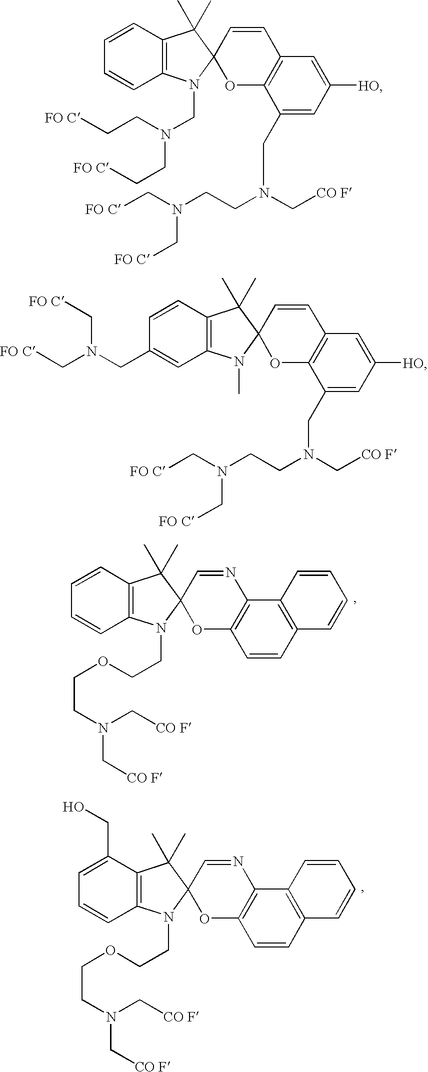

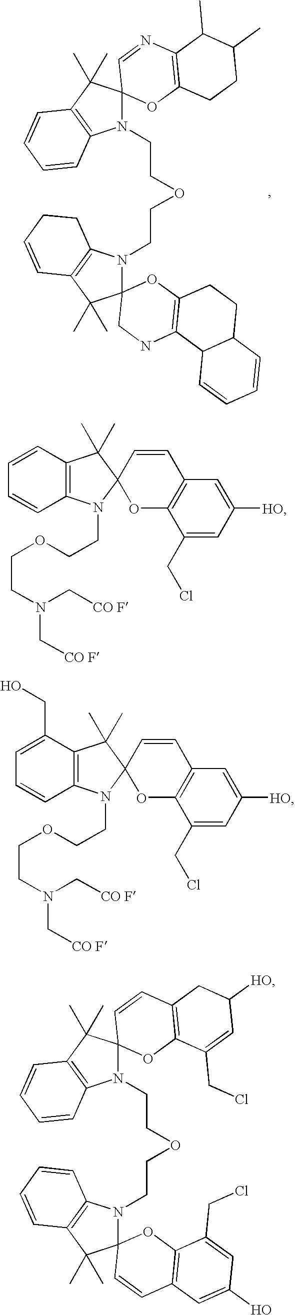

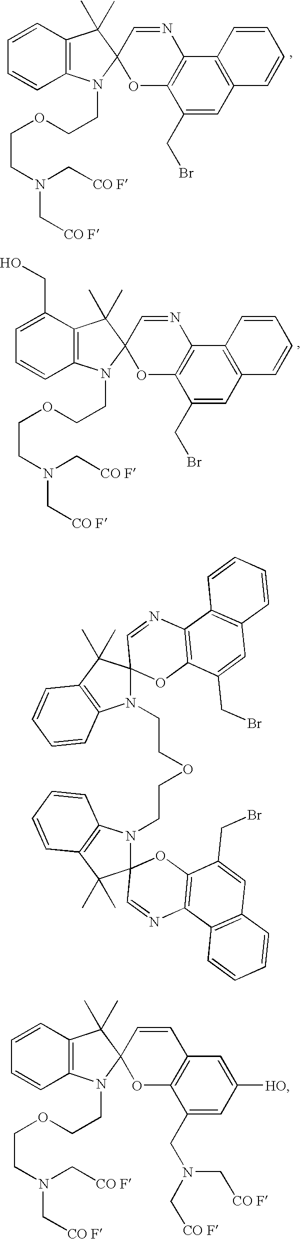

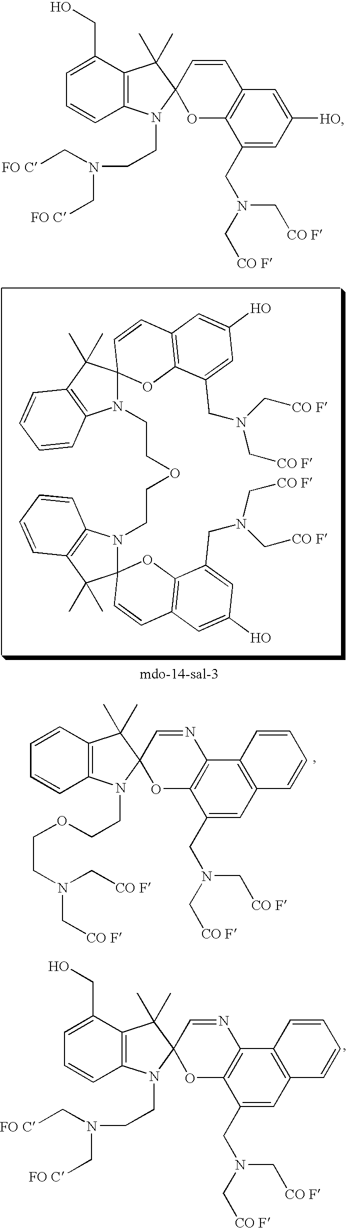

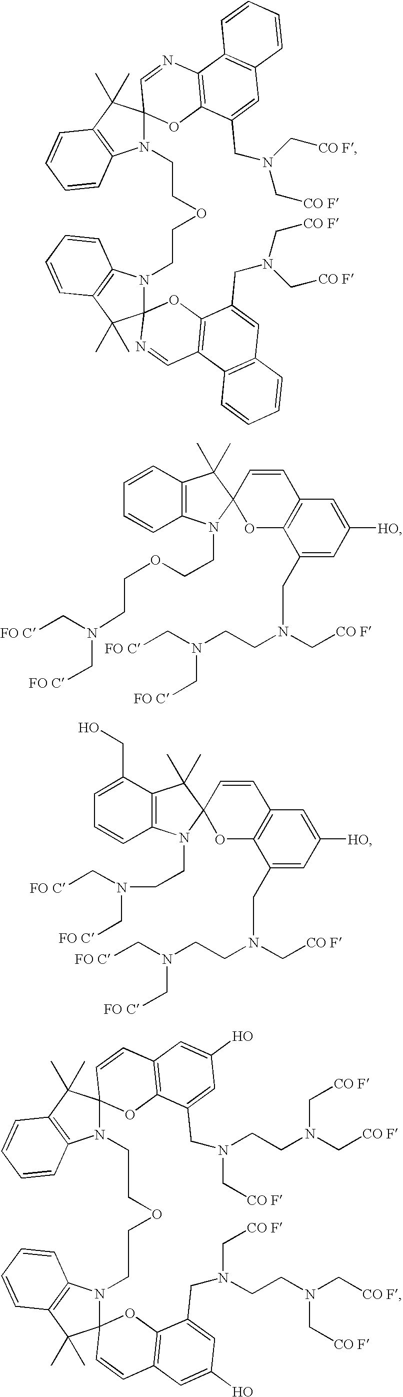

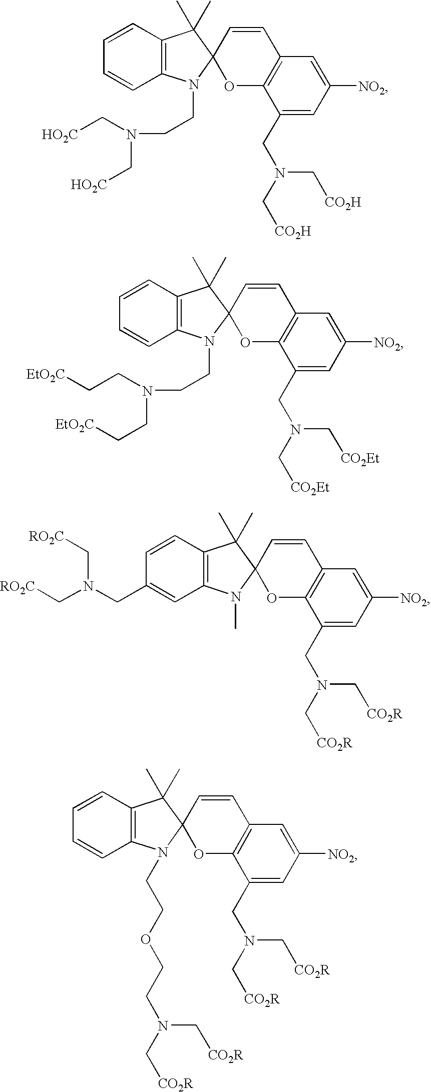

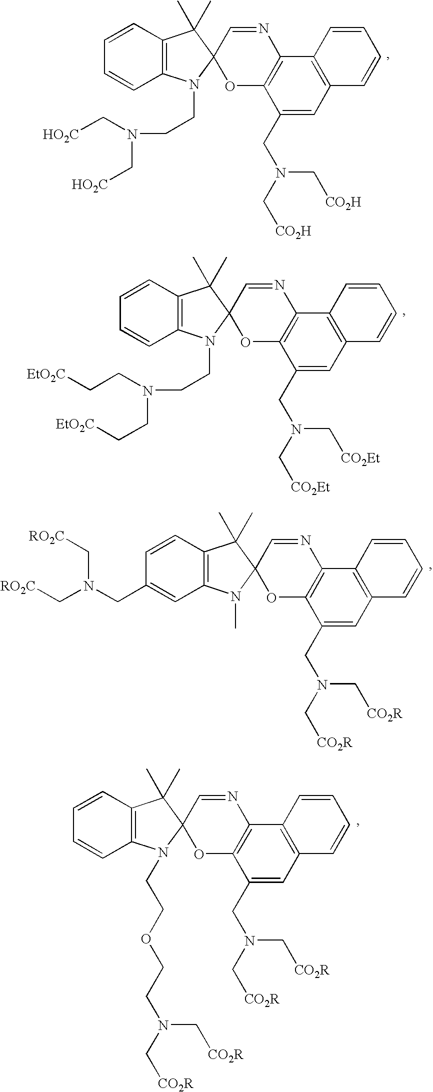

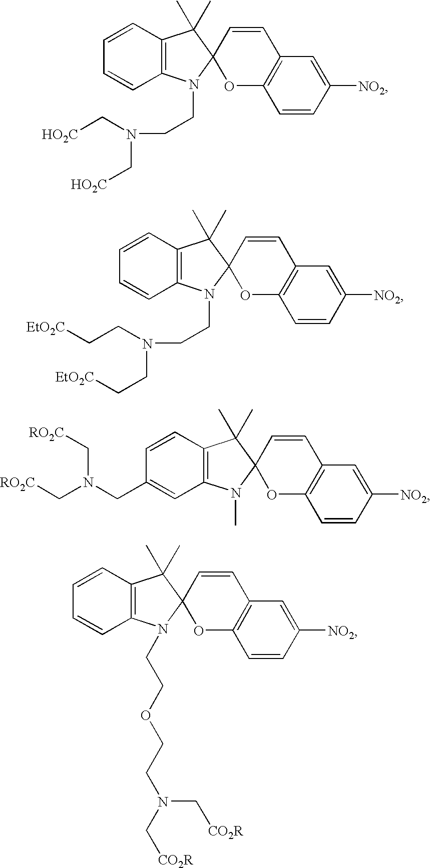

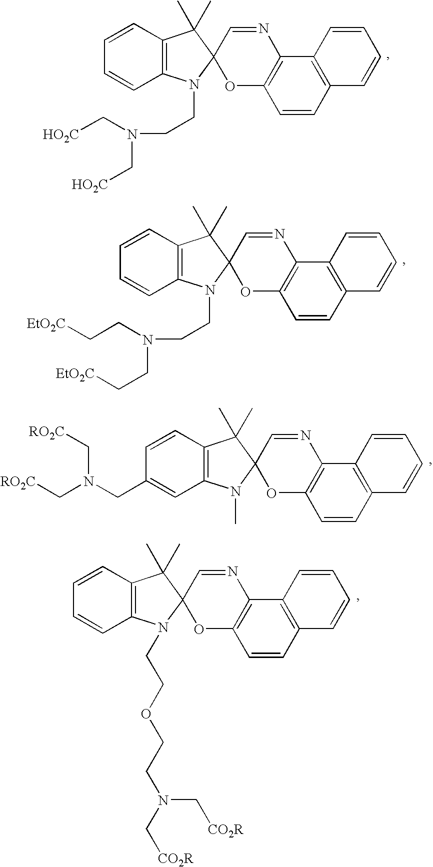

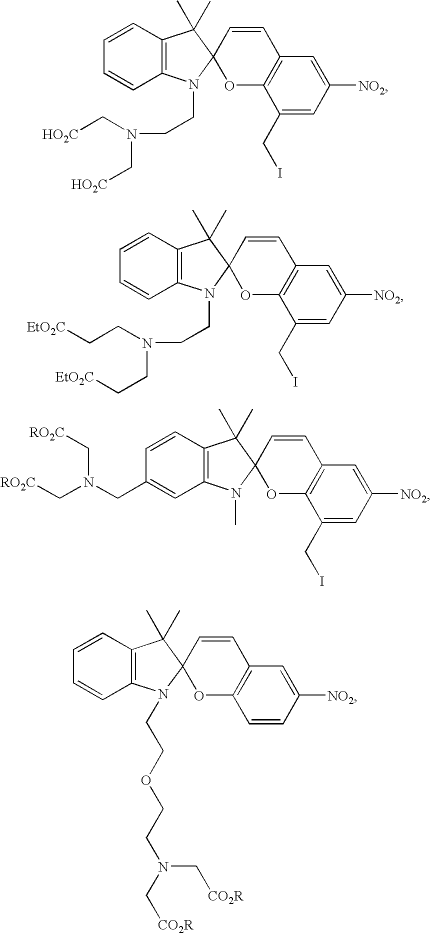

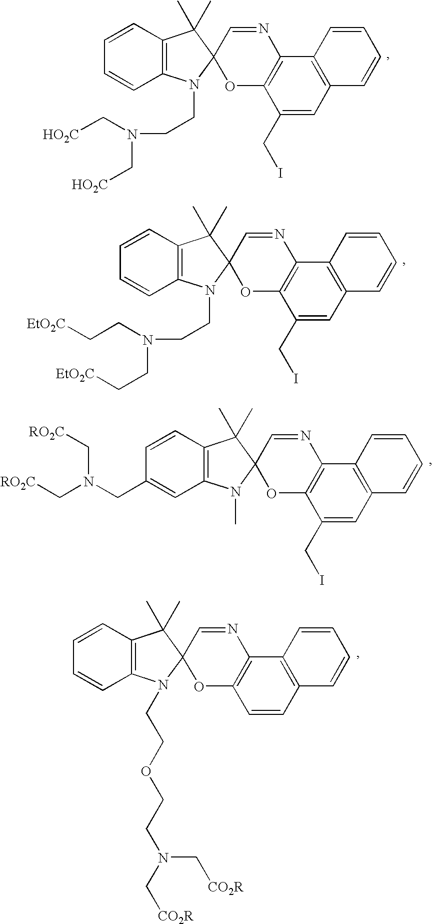

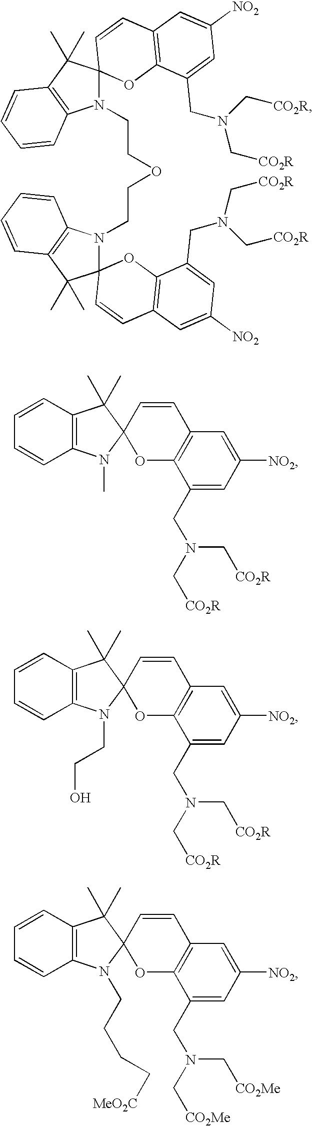

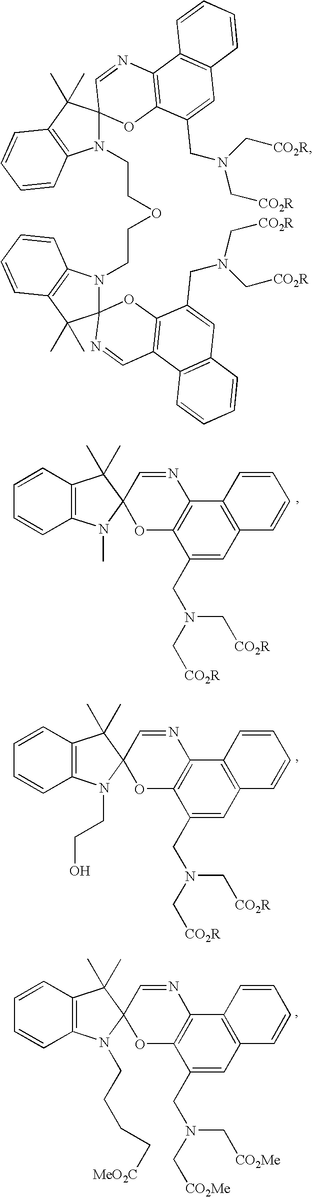

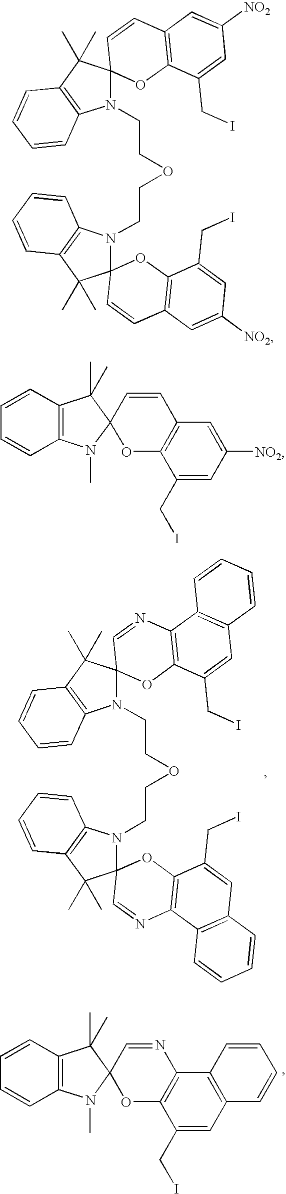

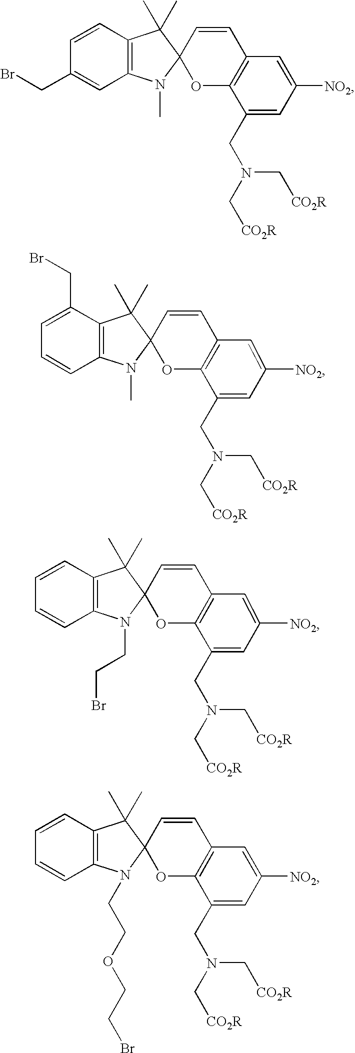

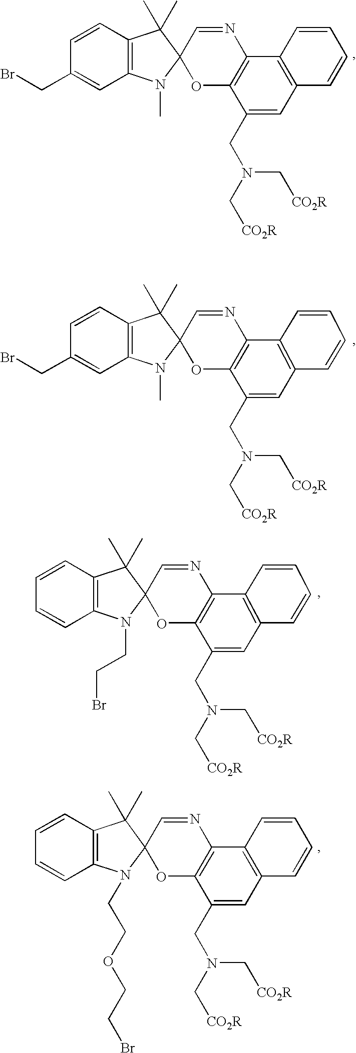

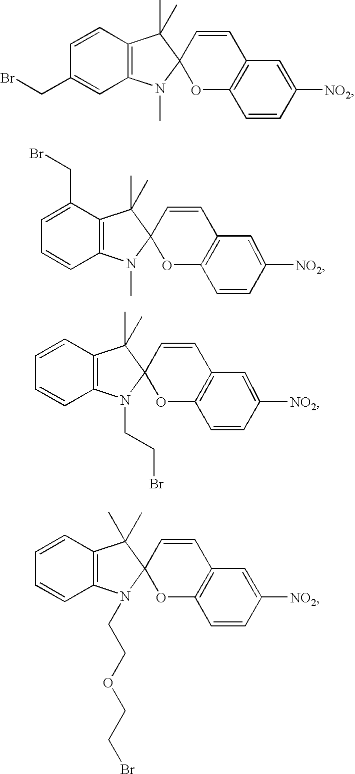

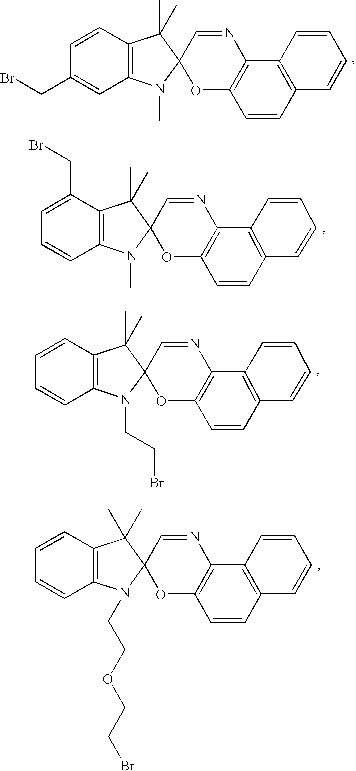

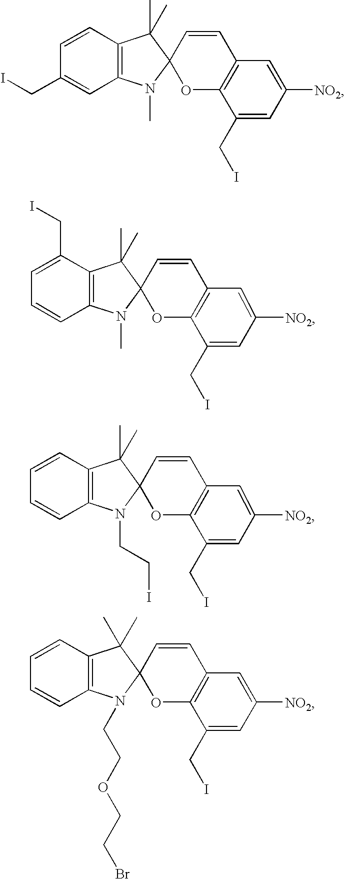

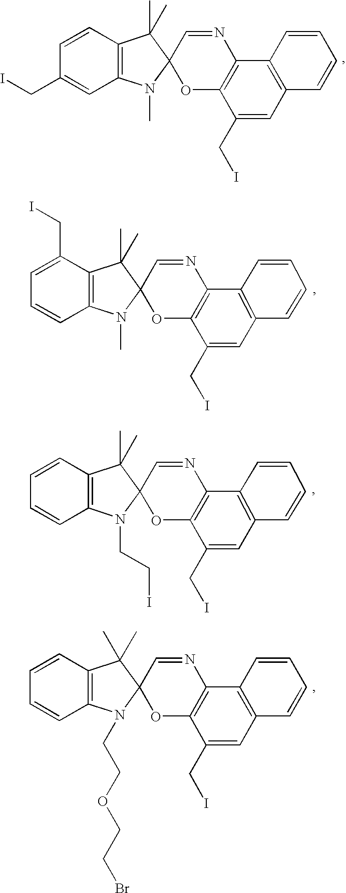

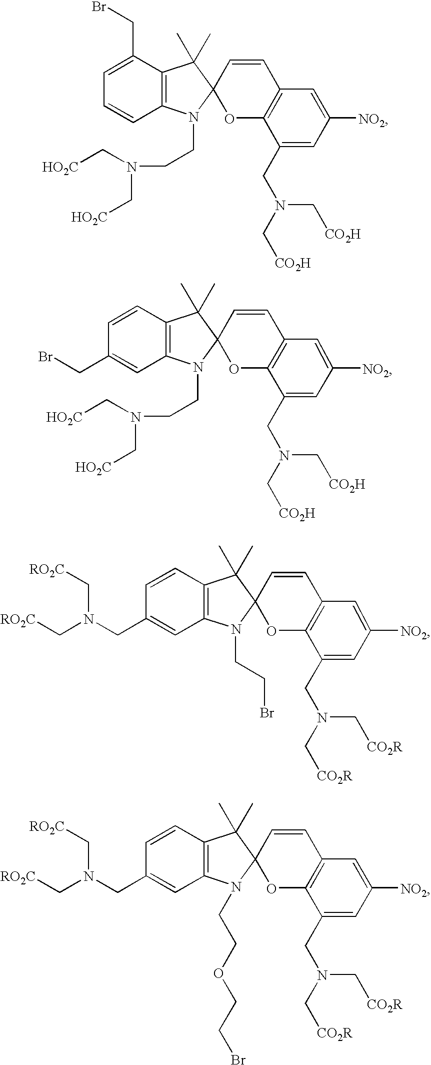

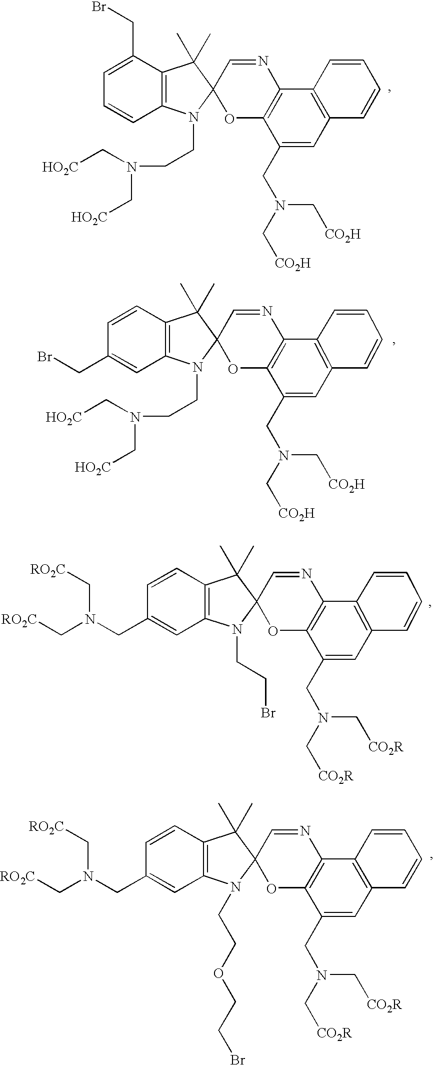

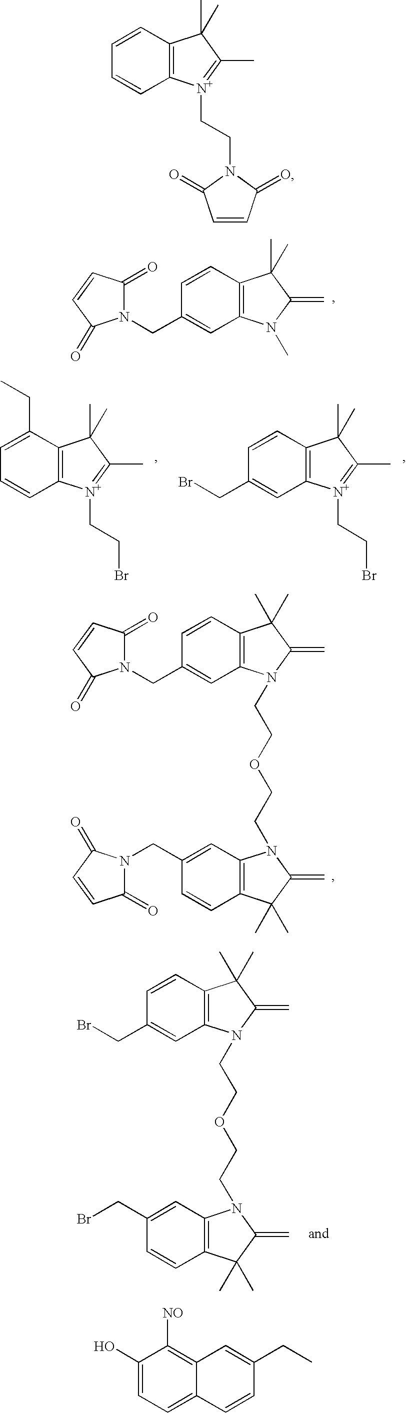

wherein R is independently selected from H, CH₃, C₂H₅ and C₃H₇.

The invention also provides a reversible optical photochromic probe comprising a compound or a derivative thereof as shown above. The probe is capable of undergoing light directed reversible transition between a first state and a second state. The first state is obtained by shining light of about 365 nm on the compound or derivative thereof, whereas the second state is obtained by shining light of about 545 nm to 620 nm on the compound or derivative thereof.

Another embodiment of the invention also provides a method of determining or controlling biomolecular interactions or activity. The method comprises the step of contacting said biomolecule with an optical photochromic probe of a compound or derivative thereof as shown above. Further, the biomolecular interactions may studied or determined using Foerster resonance energy transfer (FRET), fluorescence recovery after photobleaching (FRAP), photoactivation of fluorescence (PAF) technologies and Speckle microscopy. In this method, the optical photochromic probe is capable of undergoing light directed reversible transition between a first and second state. As discussed above, the first state is obtained by shining light of about 365 nm on the compound or derivative thereof and the second state is obtained by shining light of about 545 nm to 620 nm on the compound or derivative thereof. The biomolecules in this method include proteins, DNA, RNA, sugars, or ligands.

The present invention also provides a method of determining free or bound calcium or controlling calcium binding in a subject. The method comprises the step of contacting the subject with a reversible optical photochromic probe of a compound or a derivative thereof as shown above. The free or bound calcium determination or calcium binding is controlled by light directed reversible transition between a first state and a second state. Quantative calcium estimation and controlling calcium binding interactions may be determined using Foerster resonance energy transfer (FRET), fluorescence recovery after photobleaching (FRAP), photoactivation of fluorescence (PAF) technologies and Speckle microscopy. Further, the present invention also provides an optical photochromic probe which has at least two optical switches. Each optical switch may be independently controlled by light directed reversible transition between the first state and the second state.

The present invention also teaches a method of synthesizing a thiol reactive optical switch, comprising the steps of: (a) coupling an indoline derivative with a salycilaldehyde or nitrosonaphthol derivative to yield a spirobenzopyran or a spironaphthoxazine; and (b) conducting a halogen exchange reaction or bromination of alcohol or modified Mitsunobu reaction on the spirobenzopyran or spironaphthoxazine to yield a thiol reactive spirocompound useful as an optical switch. In this method, the indoline derivative is a compound selected from the group consisting of:

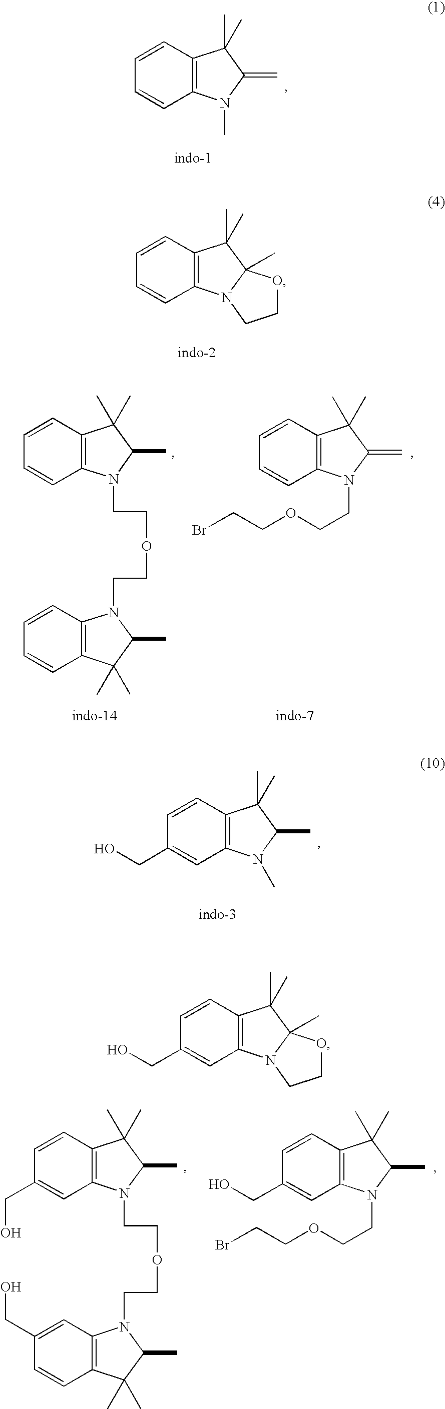

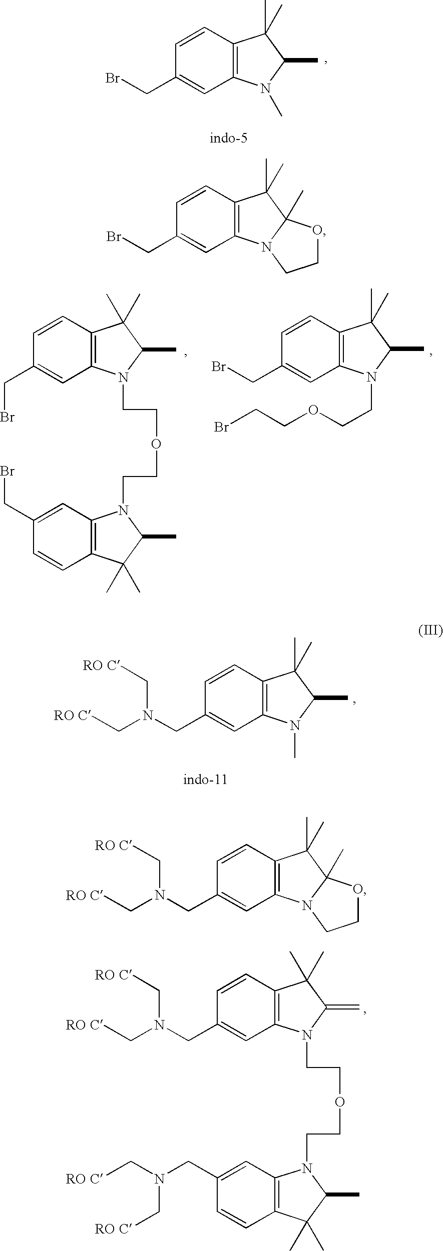

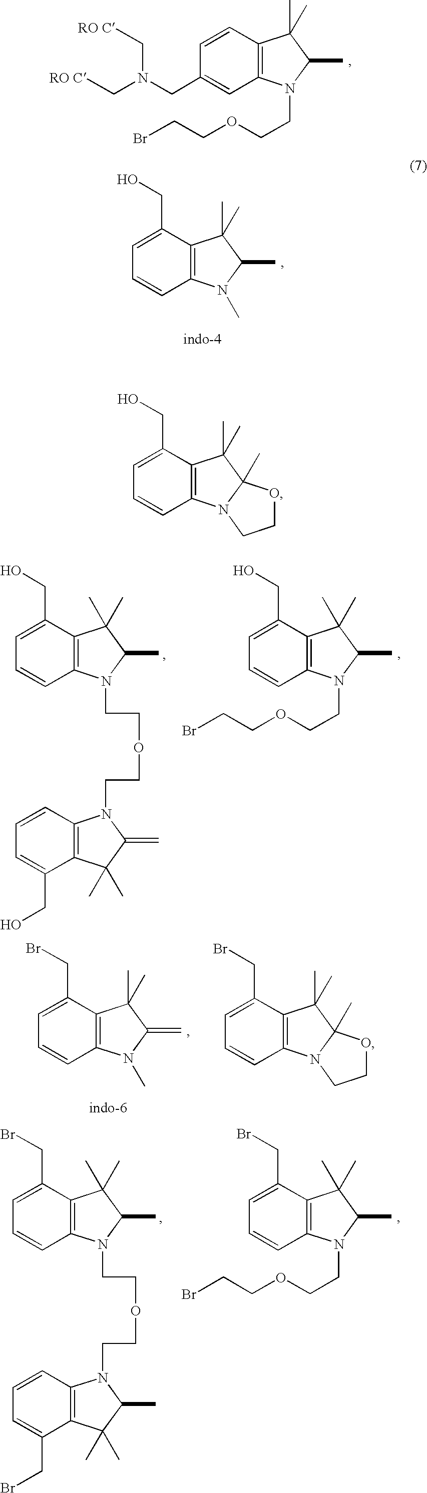

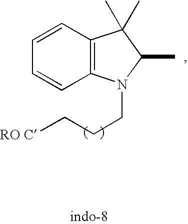

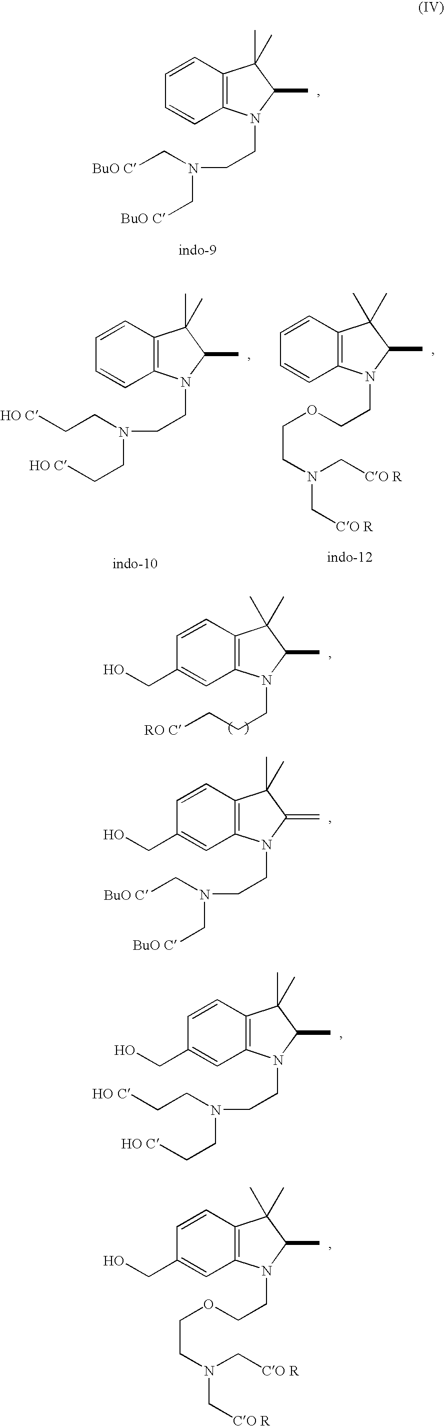

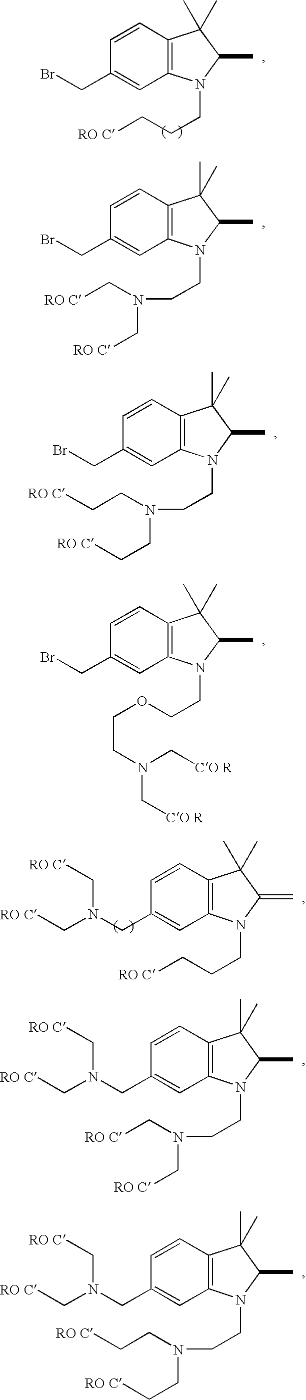

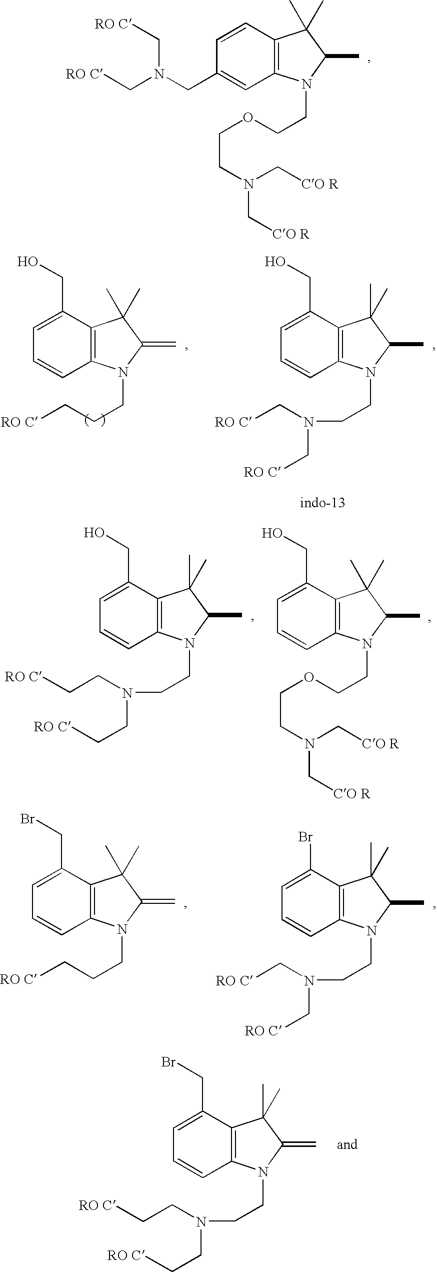

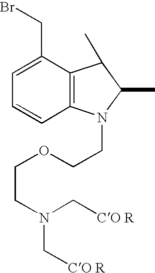

wherein R is independently selected from H, CH₃, C₂H₅ and C₃H₇.

Further, the spirobenzopyran or the spironaphthoxazine is a compound as shown described above. The indoline derivative may be synthesized by a coupling reaction of an indole derivative and an alkyl halide.

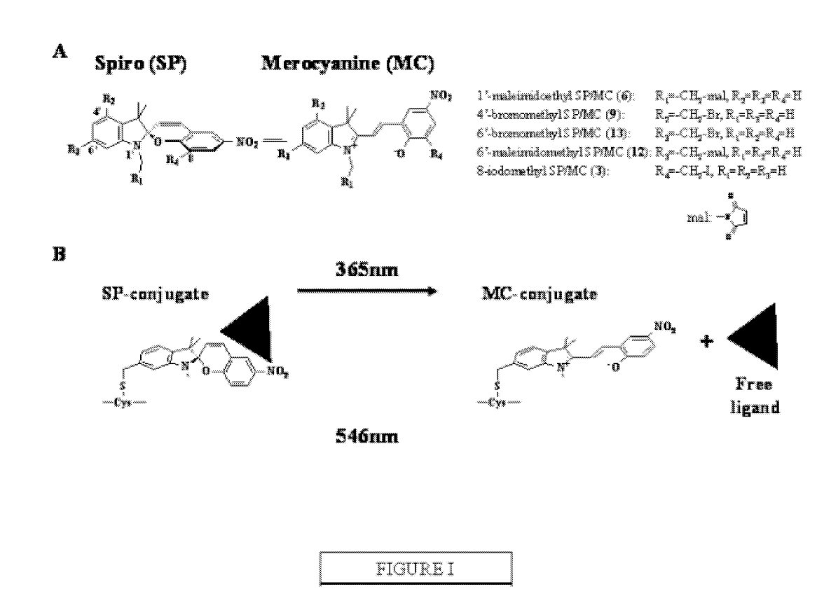

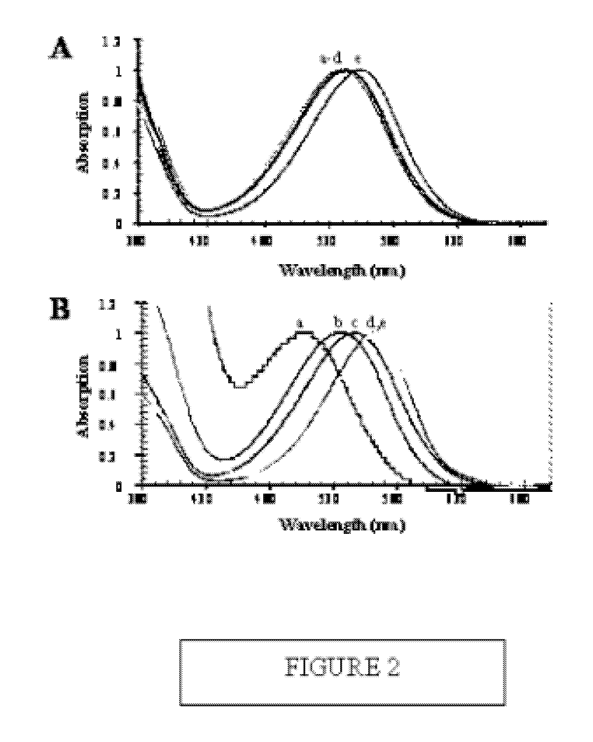

Generally, in the present invention, the inventors have shown that dipolar (protein) interactions and activities can be manipulated with unrivalled temporal and spatial resolution^{3,6} using light-directed activation of caged proteins. When coupled with FRET-based imaging this technique can provide detailed and quantitative information on thermodynamic parameters that define the regulation of specific protein complexes⁴. In one preferred embodiment, the underlying basis for the photochromic compounds, such as the spirobenzopyrans, which undergo light-directed, includes reversible transitions between a colorless spiro-(SP) state and a colorful merocyanine (MC) state¹⁰ (FIG. 1A). The excitation of the SP chromophore with 365 nm light generates MC while excitation of the MC chromophore with 546 nm light generates SP.

As part of a new approach for reversible optical switching of biomolecular interactions and activities the inventors present the design, synthesis and chemical and photochemical characterization of five spirobenzopyrans that harbor thiol reactive groups at different sites on a common spirobenzopyran scaffold. The inventors show that the colored merocyanine (MC) specifically linked to proteins acts as an environmentally sensitive probe of dipolar interactions and that the strength of the MC-protein interaction depends on the linkage geometry. Absorption spectroscopy is used to demonstrate that reversible transitions between the spiro-(SP) and MC states on proteins is achieved over many cycles using alternate irradiation with 365 nm and 546 nm light with the full and specific recovery of the MC-protein interaction. The strong interaction between MC and the protein prevent the thermal transition of MC to SP allowing specific control of the two switch states with light. The inventors believe that the higher dipole moment of the MC probe (20 D) compared to SP (5 D) will result in significant differences in the dipolar interactions of SP and MC within the bioconjugate that may be used to differentially and reversibly perturb functional interactions of the bioconjugate with ligands and other proteins.

Differently linked photochromes will exhibit a spectrum of rate constants for both the photo- and thermally driven transitions between MC and SP states. Furthermore the presence of multiple photochromes will lead to the formation of MC and SP dimers in the conjugate having different spectroscopic and photochemical properties compared to the monomer¹².

The rate for thermal conversion of MC to SP is 1000 slower for the SBP compared to the SNZ—this difference can be exploited in the design of optical switches that require a stable MC state (SBP) or unstable MC state (SNZ).

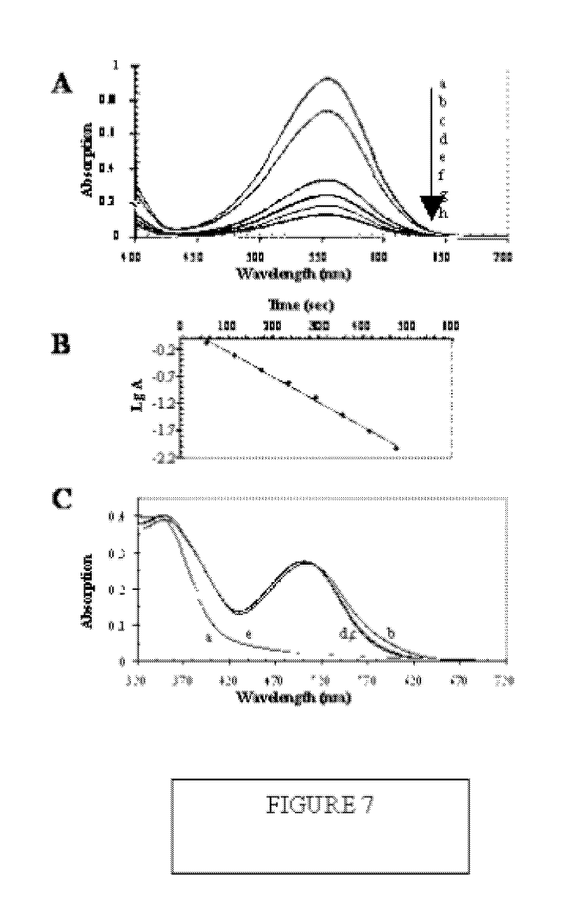

In this invention the inventors introduce a different approach for reversible, optical switching of biomolecular interactions and activity that employs chemically and spectroscopically defined conjugates harboring a single and specifically labeled photochromic probe. The inventors introduce a family of thiol reactive spirobenzopyrans that differ only in the position of the reactive group on the common chromophoric ring (FIGS. 1A, 6 and Scheme 1). Evidence supported by solvent studies suggests that the highly polarized MC ground state dipole engages in strong, linkage-specific, interactions with protein dipole. The inventors propose to exploit both the strong interaction between specifically labeled MC and the protein and the ability to modulate this interaction with 546 nm light as part of an approach to inhibit functional interactions on the protein as schematized in FIG. 1B. Dipolar MC-protein interactions also significantly reduce the rate of the thermally-driven MC-SP transition and thereby allow exclusive control of the switch states on the protein using light.

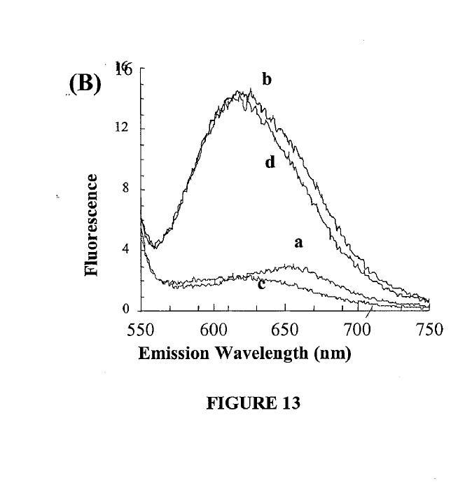

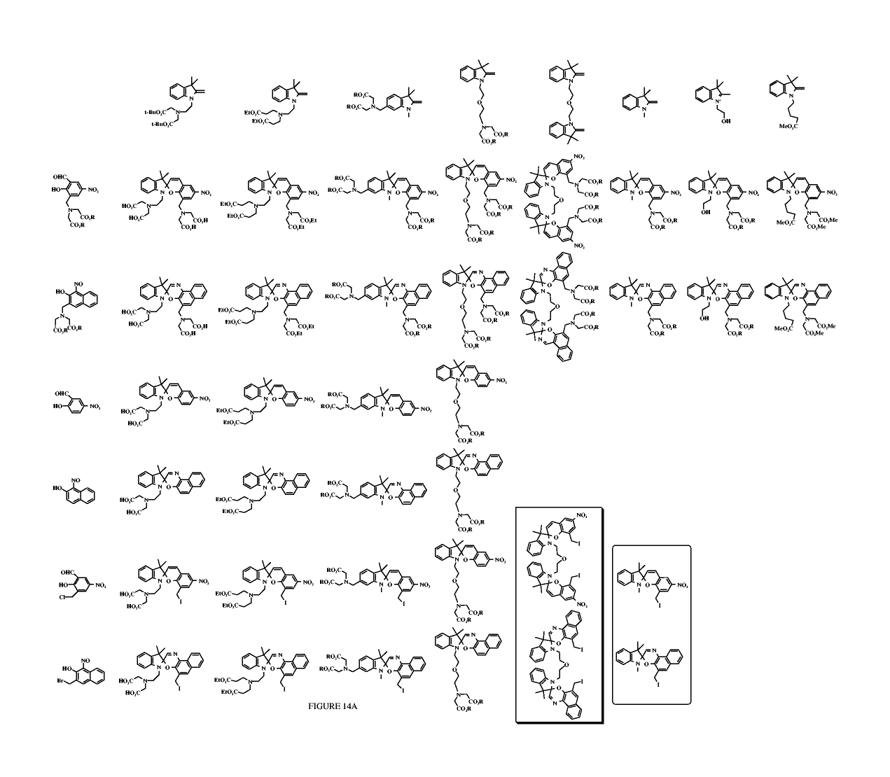

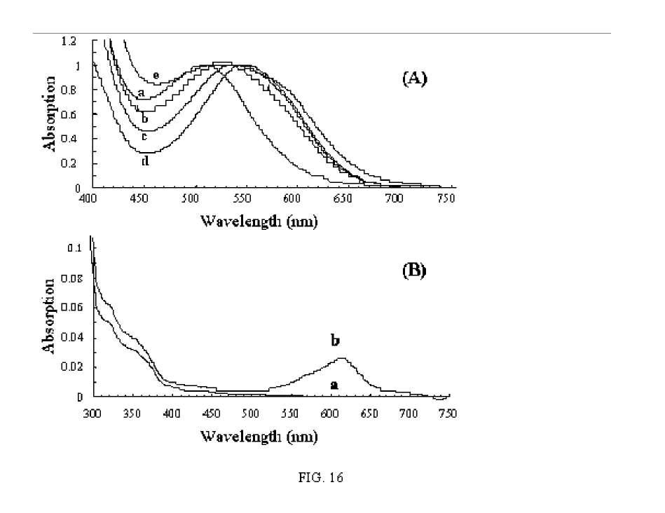

Fluorescence emission from the MC state: The fluorescence of the MC state serves as an intrinsic probe to monitor the status of the optical switch in complex environments e.g. on a surface or within a cell. The emission is centered at 620 nm for the spirobenzopyrans (SBP) probes upon excitation with 546 nm light. The same emission can be generated by uv irradiation of SBP—uv irradiation serves 2 purposes here: first it pumps the S₀—S₂ transition of MC, which emits fluorescence after returning to the S₁ state, and second it serves to maintain a constant population of MC states. As shown in FIGS. 13 and 16.

Photochromic FRET^{7,8,13} (pcFRET) uses photochromic probes to modulate the quantum yield of the donor emission. The photochromic probes described herein are more suitable probes for pcFRET based analysis of molecular proximity since they can be specifically labeled to unique cysteine residues in proteins rather than randomly introduced through lysine groups. The inventors present evidence that members of the spirobenzopyran family can be used to vary the orientation of the MC dipole in the conjugate thereby providing an experimental system to test the commonly used assumption that the dipole moments for the donor and acceptor probes are randomly orientated in space (i.e. k² is ⅔)⁹.

Certain techniques and methodologies of the present invention are described in the following examples. These examples are for illustrative purposes only and should not be deemed to narrow the scope of the present invention.

### EXAMPLE I

Materials and Methods

Instrumentation. ¹H NMR spectra were measured on a Brucker Ac 300 MHz; mass spectra were carried out on a Micromass AutoSpec for El, a Micromass LCT for ESI, or a Bruker REFLEX II for MALDI. Absorption spectra were recorded on a Hewlett-Packard 82152 diode array spectrophotometer or a Shimadzu 1601 PC instrument. Fluorescence spectroscopy was performed on an SLM-AB2 instrument (Thermoelectron, Madison, Wis.) or an ISS PC1 (Champaign, Ill.). Light-directed switching of the probes described in this work was achieved by irradiating the sample (120-1000 μL) with the 365 nm or 546 nm lines of a 100 W Hg-arc lamp (Zeiss).

Materials. The starting materials for the following syntheses are all commercially available.

Synthesis.

8-(Chloromethyl)spirobenzopyran (2) A THF solution (10 ml) of 3-chloromethyl-5-nitrosalicylaldehyde (50 mg, 0.23 mmol) and 1,3,3-trimethyl-2-methyleneindoline (40 mg, 0.23 mmol) was refluxed for 4 hours. Evaporation of the solvent gave 2 as a crude product, which was used for the subsequent reaction without further purification. MS(EI): 370(M⁺, 45), 336(72), 159(73); HRMS(EI): M⁺370.1096 (Calc. 370.1084); ¹H NMR (CDCl₃) δ1.22 (s, 3H), 1.32 (s, 3H), 2.71, (s, 3H), 4.32 (d, J=11.7 Hz, 1H), 4.38 (d, J=11.7 Hz, 1H), 5.92 (d, J=10.3 Hz, 1H), 6.55 (d, J=7.3 Hz, 1H), 6.89 (dd, J=7.3, 7.3 Hz, 1H), 6.95 (d, J=10.3 Hz, 1H), 7.09(d, J=7.3 Hz, 1H), 7.19(dd, J=7.3, 7.3 Hz, 1H), 8.00 (d, J=2.8, 1H), 8.14 (d, J=2.8 Hz, 1H).

8-(lodomethyl)spirobenzopyran (3). An acetone solution (5 ml) of crude product 2 (56 mg, ca. 0.15 mmol) and Nal (70 mg, 0.47 mmol) was stirred overnight. After evaporation, the residue was purified by column chromatography (silica gel; eluent, CH₂Cl₂) to afford 3 (55 mg, 78%). MS(EI): 335([M−I]⁺, 23), 159(13), 71 (100); HRMS: [M−I]⁺335.1385 (calc. 335.1396); ¹H NMR (CDCl₃) δ1.24 (s, 3H), 1.38 (s, 3H), 2.77, (s, 3H), 4.13 (d, J=9.3 Hz, 1H), 4.22 (d, J=9.3 Hz, 1H), 5.94 (d, J=10.4 Hz, 1H), 6.59 (d, J=7.5 Hz, 1H), 6.91 (dd, J=7.5, 7.5 Hz, 1H), 6.95 (d, J=10.4 Hz,1H), 7.12 (dd, J=1.0, 7.5 Hz, 1H), 7.21 (ddd, J=1.0, 7.5, 7.5 Hz, 1H), 7.95 (d, J=2.8, 1H), 8.08 (d, J=2.8 Hz, 1H).

1′-(Hydroxyethyl)spirobenzopyran (5). A solution of 2,3,3-trimethyl-3H-indole (1 ml, 6.3 mmol) and 2-iodoethanol (0.56 ml, 8.8 mmol) in MeCN (4 mL) was refluxed for 1 day. After being cooled to r.t., the reaction mixture was suspended in hexane, and the precipitated solid was sonicated and filtered. A part of the obtained purple solid (53 mg out of 1.37 g) was dissolved in 1N KOH (2 mL) and stirred at r.t. for 30 min. After extraction with ether, the organic layer was evaporated to afford 4 as yellow oil, which was used for the next reaction without further purification. A solution of 5-nitrososalicylaldehyde (38 mg, 0.23 mmol) and the obtained 4 in EtOH (5 mL) was refluxed for 4 hours. The mixture was evaporated and purified by column chromatography (silica gel; eluent, hexane:AcOEt=1:1) to afford purple crystal 5 (56 mg, 66% based on 2,3,3-trimethyl-3H-indole). MS(EI): 352(M⁺, 15), 337(5), 321(9), 83(100); HRMS(EI): M⁺352.1411 (calc.352.1423); ¹H NMR(CDCl₃) δ1.20 (s, 3H), 1.30 (s, 3H), 3.34 (ddd, J=5.1, 5.1, 14.7 Hz, 1H), 3.47 (ddd, J=5.5, 7.3, 14.7 Hz, 1H), 3.69-3.82 (m, 2H), 5.89 (d, J=10.5 Hz, 1H), 6.67 (d, J=7.5 Hz, 1H), 6.77 (d, J=8.5 Hz, 1H), 6.90 (dd, J=7.5, 7.5 Hz, 1H), 6.91 (d, J=10.5 Hz, 1H), 7.10 (dd, J=1.1, 7.5 Hz, 1H), 7.50 (ddd, J=1.1, 7.5, 7.5 Hz, 1H), 8.00 (d, J=2.5, 1H), 8.03 (dd, J=2.5, 8.5 Hz, 1H).

1′-(Maleimidoethyl)spirobenzopyran (6). To a dry THF (1 ml) solution of PPh₃ (25 mg, 95 μmol) was added DIAD (18 ul, 95 μmol) over 2 min at −78° C., and the reaction mixture was stirred for 5 min. To this solution, 5 (32 mg, 91 μmol) in dry THF (0.3 ml) was added over 2 min, and the mixture was stirred for 5 minutes. Neopentyl alcohol (4 mg, 4 μmol) and maleimide (9 mg, 9 μmol) were added sequentially to the reaction mixture as solids. After stirred for 5 minutes, the reaction mixture was allowed to warm up to r.t. and stirred for additional 1 hour. The reaction mixture was concentrated and then applied to preparative TLC twice (silica gel; hexane:EtOAc=2:1, then CH₂Cl₂) to afford 6 (6 mg, 15%). MS(MALDI): 432 [M+H]⁺; HRMS(ESI): [M+Na+MeOH]⁺486.1657 (calc.486.1641); ¹H NMR (CDCl₃) δ1.18 (s, 3H), 1.31 (s, 3H), 3.43 (t, J=6.7 Hz, 1H), 3.77 (t, J=6.7 Hz, 1H), 5.98 (d, J=10.4 Hz, 1H), 6.68 (dd, J=1.0, 7.5 Hz, 1H), 6.72 (s, 2H), 6.79 (d, J=10.1 Hz, 1H), 6.92 (ddd, J=1.0, 7.5, 7.5 Hz, 1H), 6.97 (d, J=10.4 Hz, 1H), 7.12 (dd, J=1.3, 7.5 Hz, 1H), 7.23 (ddd, J=1.3, 7.5, 7.5 Hz, 1H), 8.05 (d, J=2.5, 1H), 8.06 (dd, J=2.5, 10.1 Hz, 1H).

4-(Hydroxymethyl)-2,3,3-trimethyl-3H-indole (14) and 6-(hydroxymethyl)-2,3,3-trimethyl-3H-indole (15). To 3-aminobenzylalcohol (2.0 g, 16 mmol) in conc. HCl (6.4 mL) was added an aqueous solution (5.6 mL) of NaNO₂ (1.1 g, 16 mmol) at 0°. After 30 minutes, SnCl₂.2H₂O (10 g, 44 mmol) in conc. HCl (11 mL) was added to the reaction mixture. The reaction mixture was stirred for an additional 30 minutes, then washed with ether, neutralized with NaOH, and extracted with ether. The ether extract was evaporated to afford 3-hydrazinobenzyl alcohol, which was used for the next reaction without further purification. The obtained 3-hydrazinobenzyl alcohol was dissolved in EtOH (10 mL) and refluxed with 3-methyl-2-butanone (1.7 ml, 16 mmol) and concentrated H₂SO₄ (1 ml) for 17 hours. After concentration, the reaction mixture was washed with CH₂Cl₂, basified with Sat. Na₂CO₃, and extracted with CH₂Cl₂. The CH₂Cl₂ extract was subjected to column chromatography (silica gel; eluent, EtOAc) and preparative TLC (silica gel; EtOAc) to give 14 (86 mg, 3% based on 3-aminobenzyl alcohol) and the isomer 15 (131 mg, 4%). 14: MS(EI): 189 (M⁺, 88), 174 (48), 156 (36), 83 (100); HRMS(EI): M⁺189.1161 (calc. 189.1154): ¹H NMR (CDCl₃) δ1.43(s, 6H), 2.29(s, 3H), 4.88(2H, s), 7.26 (d, J=7.5 Hz, 1H), 7.35 (dd, J=7.5 Hz, 1H), 7.50 (d, J=7.5 Hz, 1H). 15: MS(EI): 189(M⁺, 100), 174 (56), 158(19), 144(18); HRMS(EI): M⁺189.1155 (calc.189.1154); ¹H NMR (CDCl₃) δ1.29(s, 6H), 2.27(s, 3H), 4.73(s, 2H). 7.22 (d, J=7.6 Hz, 1H), 7.25 (d, J=7.6 Hz, 1H), 7.54 (s, 1H).

4′-(Hydroxymethyl)spirobenzopyran (8). A solution of 14 (51 mg, 0.27 mmol) and CH₃I (0.15 ml, 2.4 mmol) in CH₂Cl₂ (1 mL) was refluxed for overnight. The reaction mixture was filtered, and the filtrate was dissolved in 0.5 N KOH (1 ml) and stirred for 15 min. After extraction with CH₂Cl₂, the extract was evaporated to afford oil 7 as a crude product. With 5-nitrosalicylaldehyde (45 mg, 0.27 mmol), 7 was stirred in EtOH (2 mL) at r.t. over night. The reaction mixture was evaporated and subjected to column chromatography (silica gel; eluent, EtOAc) to afford 8 (34 mg, 36% based on 14). MS(EI): 352 (M⁺, 22), 337 (4), 189 (10), 83 (100); HRMS(EI): M⁺352.1408 (calc.352.1423); ¹H NMR (CDCl3) 1.25 (s, 3H), 1.28 (s, 3H), 2.74 (s, 3H), 4.74 (d, J=12.5 Hz, 1H), 4.82 (d, J=12.5 Hz, 1H), 5.85 (d, J=10.4 Hz, 1H), 6.55 (d, J=7.7 Hz, 1H), 6.77 (d, J=9.0 Hz, 1H), 6.93 (d, J=7.7 Hz, 1H), 6.96 (d, J=10.4 Hz, 1H), 7.2 3 (dd, J=7.7, 7.7 Hz, 1H), 8.01 (d, J=2.8, 1H), 8.02 (dd, J=2.8, 9.0 Hz, 1H).

4′-(Bromomethyl)spirobenzopyran (9). To a THF solution (1 mL) of 8 (34 mg, 97 μmol) and CBr₄, (70 mg, 211 μmol) was dropped Ph₃P (50 mg, 191 μmol) at 0°. The reaction mixture was stirred at 0° for 30 minutes and at r.t. overnight. After evaporation of the reaction mixture, the residue was subjected to column chromatography (silica gel; eluent, hexane:EtOAc=5:1) to afford 9 (24 mg, 60%). MS(EI): 416 (76), 414 (M⁺, 74), 335 (41), 85 (100), 83 (99); HRMS(EI): M⁺414.0580 (calc.414.0579); ¹H NMR(CDCl₃) 1.25 (s, 3H), 1.32 (s, 3H), 2.73 (s, 3H), 4.51 (d, J=10.3 Hz, 1H), 4.65 (d, J=10.3 Hz, 1H), 5.84 (d, J=10.1 Hz, 1H), 6.51 (d, J=7.8 Hz, 1H), 6.79 (d, J=8.5 Hz, 1H), 6.87 (d, J=7.8 Hz, 1H), 6.97 (d, J=10.1 Hz, 1H), 7.20 (dd, J=7.8, 7.8 Hz, 1H), 8.01 (s, 1H), 8.03 (dd, J=2.8, 8.5 Hz, 1H).

6′-(Hydroxymethyl)spirobenzopyran (11). A solution of 15 (50 mg, 0.27 mmol) and CH₃I (100 ul, 1.6 mmol) in CH₂Cl₂ (2 mL) was refluxed for 12 hours. The reaction mixture was filtered, and the filtrate was dissolved in 0.5 N NaOH and stirred for 15 min. After extraction with CH₂Cl₂, the extract was evaporated to afford oil 10 as a crude product. With 5-nitrosalicylaldehyde (50 mg, 0.30 mmol), 10 was refluxed in EtOH (2 mL) for 2 hours, and then the reaction mixture was evaporated and subjected to column chromatography (silica gel; eluent, EtOAc) to afford 11 (70 mg, 75% based on 15). MS(EI): 352 (M⁺, 10), 337 (4), 83 (100); HRMS(EI): M⁺352.1434 (calc.352.1423); ¹H NMR (CDCl3) 1.19 (s, 3H), 1.30 (s, 3H), 2.76 (s, 3H), 4.70 (s, 2H), 5.87 (d, J=10.5 Hz, 1H), 6.62 (s, 1H), 6.77 (d, J=8.7 Hz, 1H), 6.94 (d, J=10.5 Hz, 1H), 7.07 (d, J=7.2 Hz, 1H), 8.01 (d, J=2.4, 1H), 8.02 (dd, J=2.4, 8.7 Hz, 1H).

6′-(Maleimidomethyl)spirobenzopyran (12). To a dry THF (1 ml) solution of PPh₃ (25 mg, 95 μmol), was added DIAD (18 ul, 95 μmol) over 2 minutes at −78° C., and the reaction mixture was stirred for 5 minutes. To this solution, 6′-(hydroxymethyl)spirobenzopyran 11 (34 mg, 97 μmol) in dry THF (0.3 ml) was added over 2 minutes, and the mixture was stirred for 5 minutes. Neopentyl alcohol (4 mg, 4 μmol) and maleimide (9 mg, 9 μmol) were added sequentially to the reaction mixture as solids. After stirred for 5 minutes, the reaction mixture was allowed to warm up to r.t. and stirred for additional 1 h. The reaction mixture was concentrated and then applied to preparative TLC (silica gel; hexane:EtOAc=1:1) to afford 12 (10 mg, 24%). MS(EI): 431 (M⁺, 10), 416 (3), 268 (8), 83 (100); HRMS(EI) M⁺431.1497 (calc.431.1481); ¹H NMR (CDCl₃) δ1.61 (s, 3H), 1.27 (s, 3H), 2.74 (s, 3H), 5.84 (d, J=10.2 Hz, 1H), 6.54 (s, 1H), 6.73 (s, 2H), 6.78 (d, J=8.6 Hz, 1H), 6.88 (d, J=7.6 Hz, 1H), 6.92 (d, J=10.2 Hz, 1H), 7.02 (d, J=7.6 Hz, 1H), 8.01 (d, J=2.4, 1H), 8.03 (dd, J=2.4, 8.6 Hz, 1H).

6′-(Bromomethyl)spirobenzopyran (13). To a THF solution (1.5 mL) of 11 (28 mg, 78 μmol) and CBr₄, (53 mg, 160 μmol) was dropped a THF solution (0.5 mL) of Ph₃P (42 mg, 160 μmol) at 0 C. The reaction mixture was stirred at 0° for 30 min and at r.t. overnight after evaporation of the reaction mixture, the residue was subjected to column chromatography (silica gel; eluent, hexane:EtOAc=5:1) to afford 13 (18 mg, 55%) with recovered 11 (10 mg, 36%). MS(EI): 416 (1), 414(M⁺, 1), 335 (2); HRMS(EI): M⁺414.0577 (calc.414.0579); ¹H NMR(CDCl₃) 1.19 (s, 3H), 1.29 (s, 3H), 2.76 (s, 3H), 4.52 (d, J=10.5 Hz, 1H), 4.56 (d, J=10.5 Hz, 1H), 5.86 (d, J=10.2 Hz, 1H), 6.58 (d, J=1.3 Hz, 1H), 6.79 (d, J=8.4 Hz, 1H), 6.92 (dd, J=1.3, 7.3 Hz, 1H), 6.94 (d, J=10.3 Hz, 1H), 7.04 (d, J=7.3 Hz, 1H), 8.01 (d, J=3.2, 1H), 8.04 (dd, J=3.2, 8.4 Hz, 1H).

6′-(Hydroxymethyl)spironaphthoxazin (16). A solution of 1 5 (62 mg, 0.33 mmol) and CH₃I (150 μl, 2.4 mmol) in CH₂Cl₂ (1 mL) was refluxed for 12 hours. The reaction mixture was filtered, and the filtrate was dissolved in 0.5 N NaOH and stirred for 15 min. After extraction with CH₂Cl₂, the extract was evaporated to afford oil 10 (43 mg) as a crude product. With 1-nitroso-2-naphthol (39 mg, 0.23 mmol), 10 was refluxed in EtOH (10 mL) for 3 hours, and then the reaction mixture was evaporated and subjected to column chromatography (silica gel; hexane:EtOAc 3:1) and preparative TLC (silica gel; hexane:EtOAc 2:1) to afford 16 (43 mg, 53% based on 15). MS(EI): 358(M⁺, 20), 343(15), 189(20), 83 (100); HRMS(EI): M⁺358.1670 (calc.358.1681); ¹H NMR(CDCl3) 1.35 (s, 3H), 1.36 (s, 3H), 2.78 (s, 3H), 4.70 (m, 2H), 6.63 (s, 1H), 6.88 (d, J=7.3 Hz, 1H), 7.01 (d, J=8.9 Hz, 1H), 7.07 (d, J=7.3 Hz, 1H), 7.40 (dd, J=7.9, 7.9 Hz, 1H), 7.58 (dd, J=7.9, 7.9 Hz, 1H), 7.67 (d, J=8.9 Hz, 1H), 7.75 (s, 1H), 7.75 (d, J=7.9 Hz, 1H), 8.56 (d, J=7.9 Hz, 1H)

6′-(Bromomethyl)spironaphthoxazin (17). To a THF solution (1 mL) of 16 (16 mg, 45 μmol) and CBr₄, (30 mg, 91 μmol) was dropped a THF solution (0.5 mL) of Ph₃P (23 mg, 88 μmol) at 0 C. The reaction mixture was stirred at 0° for 30 min and at r.t. overnight. after evaporation of the reaction mixture, the residue was subjected to preparative TLC (silica gel; hexane:EtOAc=2:1) to afford 17 (3 mg, 15%) with recovered 16 (11 mg, 69%). MS(EI): 422 (6), 420 (M⁺, 6), 407(4), 405(4), 199(40), 83(100); HRMS(EI): M⁺420.0839 (calc. 420.0837); ¹H NMR (CDCl₃) 1.33 (s, 3H), 1.35 (s, 3H), 2.77 (s, 3H), 4.51 (d, J=10.2 Hz, 1H), 4.55 (d, J=10.2 Hz, 1H), 6.59 (d, J=1.6 Hz, 1H), 6.92 (dd, J=1.5, 7.4 Hz 1H), 7.00 (d, J=9.2 Hz, 1H), 7.03 (d, J=7.9 Hz, 1H), 7.40 (ddd, J=1.1, 7.0, 8.2 Hz, 1H), 1H), 7.58 (ddd, J=1.5, 7.0, 8.2 Hz, 1H), 7.67 (d, J=9.2 Hz, 1H), 7.73 (s, 1H), 7.75 (d, J=8.2 Hz, 1H), 8.55 (d, J=8.2 Hz, 1H)

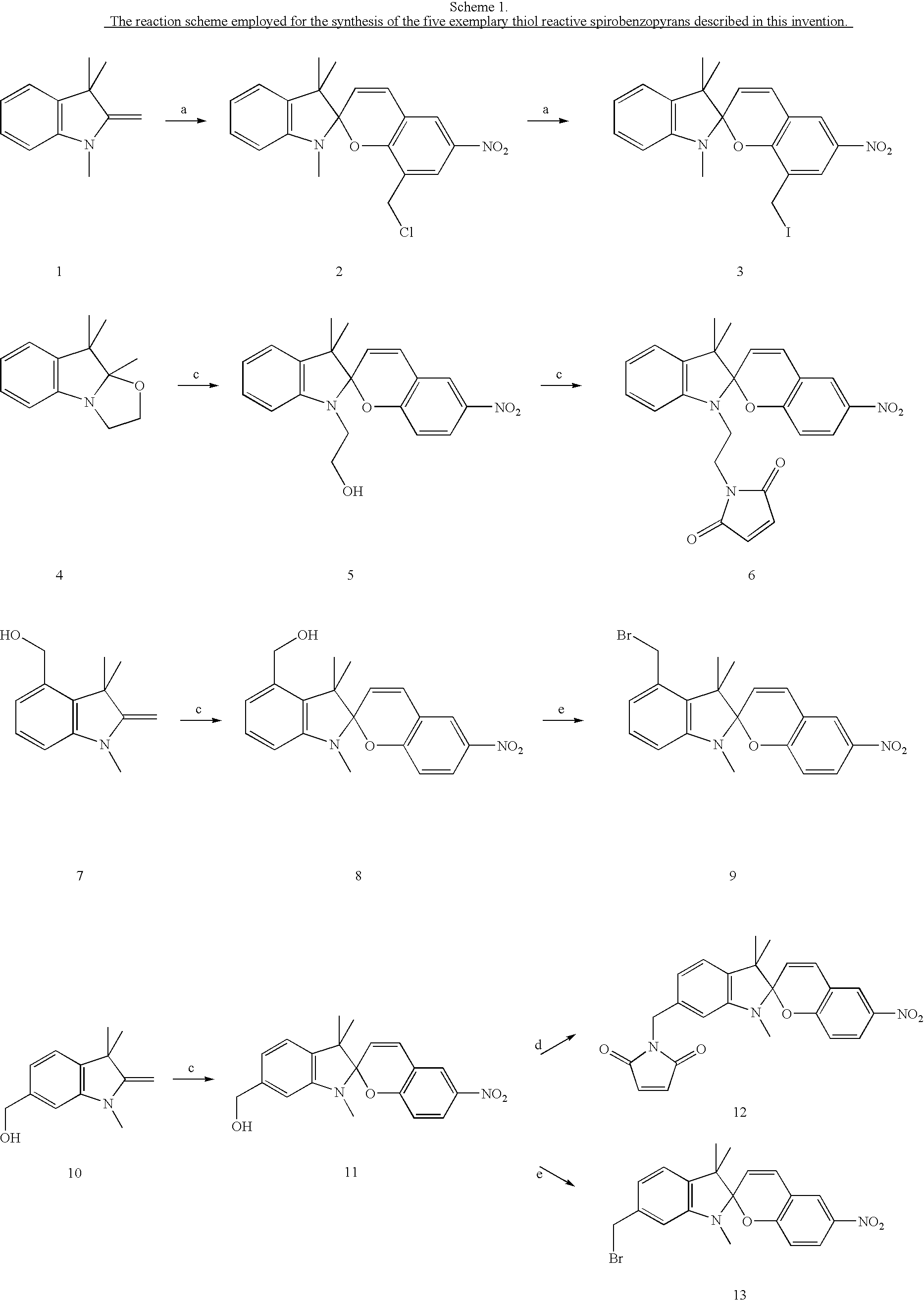

Reagents: (a) 3-chloromethyl-5-nitrosalicylaldehyde, THF; (b) Nal, acetone; (c) 5-nitrosalicylaldehyde, EtOH; (d) PPh₃, DIAD, maleimide, neopentyl alcohol, THF; (e) PPh₃, CBr₄, THF.

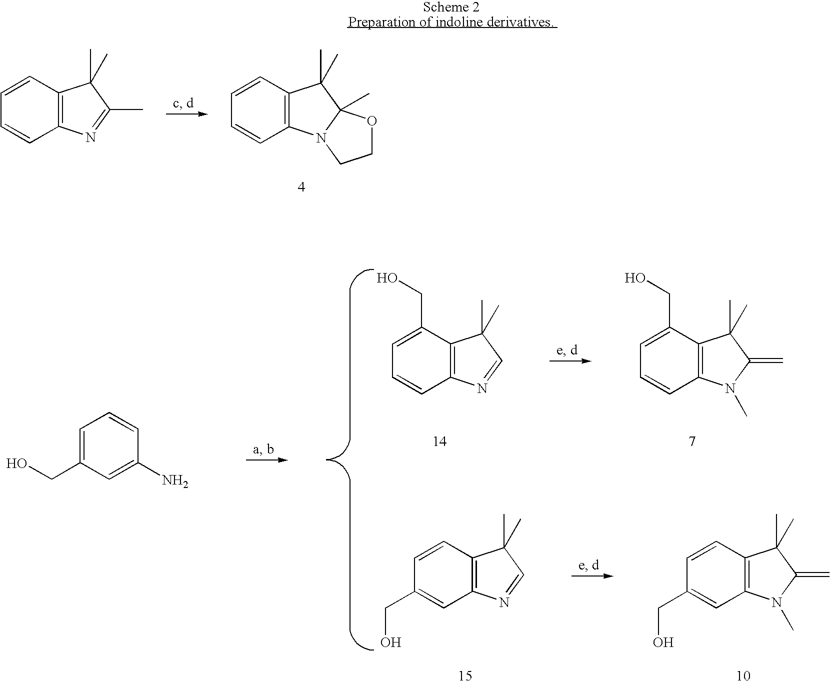

Reagents: (a) NaNO₂, SnCl₂.H₂O, HCl (b) 3-methyl-2-butanone, H₂SO₄, EtOH; (c) 2-iodoethanol, CH₃CN; (d) KOH, H₂O (e) Mel, CH₂Cl₂.

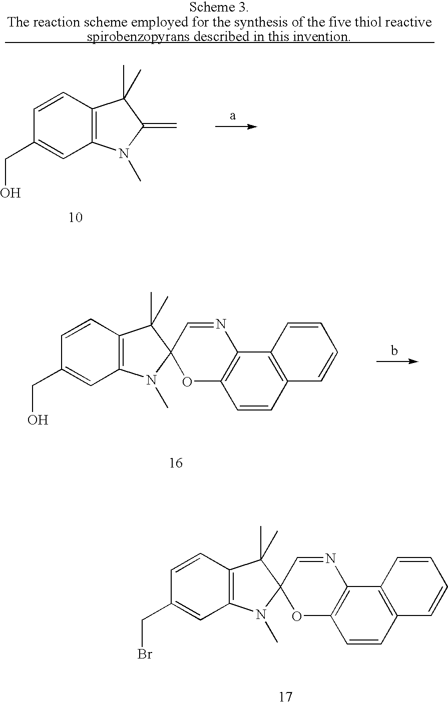

Reagents: (a) 1-nitroso-2-naphthol, EtOH; (b) PPh₃, CBr₄, THF.

Chelate-Spirocompounds

6-(bromomethyl)-2,3,3-trimethyl-3H-indole (1).

To a CH₂Cl₂ solution (2.0 mL) of 15 (35 mg, 185 μmol) and NBS, (40 mg, 220 μmol) was dropped Ph₃P (58 mg, 220 μmol) at 0° C. The reaction mixture was stirred at 0° C. for 1 hr and at r.t. 3 h. After evaporation of the reaction solvent, the residue was subjected to preparative TLC (silica gel, EtOAc) to afford I (42 mg, 89%). MS(EI): 253(7), 251(M⁺, 7), 172(M⁺-Br, 62), 83(100); HRMS(EI): M⁺251.0320 (calc. 251.0310); ¹H NMR (CDCl₃) δ1.31(s, 6H), 2.30(s, 3H), 4.57(s, 2H), 7.25-7.28(m, 2H), 7.56(s, 1H).

6-[N′,N′-bis(tert-buthyloxycarbonylmethyl)aminomethyl]-2,3,3-trimethyl-3H-indole (II). To a THF (12 ml) suspension of di-tert-butyl iminodiacetate (203 mg, 835 μmol) and ground K₂CO₃ (126 mg, 913 μmol) was added a THF (2 ml) solution of I (42 mg, 116 μmol) dropwise at refluxing condition, and the reaction mixture was further refluxed for 6 h. After evaporation of the reaction solvent, the residue was subjected to preparative TLC (silica gel, hexane:EtOAc 3:1) to afford I (46 mg, 67%). MS(EI): 31 5(M⁺—CO₂^{t}Bu,55), 215(53), 196(80), 172(100); HRMS(EI): [M-^{t}BuO₂C]⁺315.2076 (calc.315.2073); ¹H NMR (CDCl₃) δ1.29(s, 6H), 1.47(s, 18H), 2.27(s, 3H), 3.44(s, 4H), 3.94(s, 2H), 7.22(d, J=7.6 Hz, 1H), 7.32(dd, J=1.3, 7.6 Hz, 1H), 7.49(d, J=1.3 Hz, 1H).

### 3-[N,N-bis(tert-buthyloxycarbonylmethyl)aminomethyl]-5-nitrosalicylaldehyde (V)

To a THF (8 ml) solution of di-tert-butyl iminodiacetate (504 mg, 2.1 mmol) and Et₃N (0.54 ml, 3.9 mmol) was added a THF (2 ml) solution of 3-chloromethyl-5-nitrosalicylaldehyde (435 mg, 2 mmol) dropwise at refluxing condition, and the reaction mixture was further refluxed for 4 h. After filtration, the reaction solvent was evaporated to afford V as a mixture with Et₃N (10:7), which was used for the next reaction without purification. ¹H NMR(CDCl₃) δ1.50(s, 18H), 3.46(s, 4H), 4.07(s, 2H), 8.22(d, J=3.8 Hz, 1H), 8.65(d, J=3.8 Hz, 1H), 10.44 (s, 1H)

### 8,6′-BIPS-tetraester (VI)

A solution of II (20 mg, 48 μmol) and CH₃I (0.1 ml, 1.6 mmol) in CHCl₃ (1.5 mL) was heated at 65° C. for 14 h. To the reaction mixture 0.5 N NaOH (1 ml) was added and stirred for 15 min. After extraction with CH₂Cl₂, the extract was evaporated to afford oil III as a crude product, which was used for the next reaction without purification and characterization. A solution of crude V (25 mg containing Et₃N, ca. 51 μmmol) and the obtained III in THF (2 mL) was stirred for over night. The mixture was evaporated and purified by column chromatography (Sephadex; eluent, hexane:MeOH: CH₂Cl₂=2:1:1) to afford purple crystal VI (6 mg, 15% based on II). HRMS(ESI): [M+Na]⁺589.4429 (calc.859.4469); ¹H NMR (CDCl₃) δ1.18 (s, 3H), 1.26 (s, 3H), 1.39 (s, 18H), 1.49 (s, 18H), 2.70 (s, 3H), 3.23 (d, J=17.0 Hz, 2H), 3.29 (d, J=17.0 Hz, 2H), 3.46 (s, 4H), 3.59 (d, J=15.8 Hz, 1H), 3.66 (d, J=15.8 Hz, 1H), 3.87 (s, 2H), 5.84 (d, J=10.4 Hz, 1H), 6.63 (s, 1H), 6.81 (dd, J=1.4, 7.4 Hz, 1H), 6.91 (d, J=10.4 Hz, 1H), 6.97 (d, J=7.4 Hz, 1H), 7.92 (d, J=2.5 Hz, 1H), 8.30 (d, J=2.5, 1H).

### 8,6′-BIPS-TA (VII)

To a CH₂Cl₂ (0.3 ml) of VI (2.5 mg, 3.0 μmol) was added trifluoroacetic acid (TFA) (0.5 ml) and the reaction mixture was stirred at r.t. for 5 h. After evaporation of the solvent and TFA, the residue was subjected to column chromatography (Sephadex; eluent, hexane:MeOH: CH₂Cl₂=2:1:1) to afford VII (1 mg, 55%). ¹H NMR (CD₃OD) δ1.24-1.28 (m, 6H), 2.80 (s, 3H), 3.34 (s, 2H), 3.47 (s, 4H), 4.01 (s, 4H), 4.45 (s, 2H), 6.01 (d, J=10.3 Hz, 1H), 6.73 (s, 1H), 6.93 (d, J=7.6 Hz, 1H), 6.93 (d, J=10.3 Hz, 1H), 7.13 (d, J=10.3 Hz, 1H), 7.19 (d, J=7.6 Hz, 1H), 8.09 (d, J=2.7 Hz, 1H), 8.26 (d, J=2.7, 1H).

### 8,1′- BIPS-tetraester (VIII)

A MeCN (0.8 mL) solution of 2,3,3-trimethyl-3H-indole (16 mg, 0.1 mmol) and N,N-bis(tert-buthyloxycarbonylmethyl)-2-bromoethylamine (35 mg, 0.1 mmol), which was prepared according to xxx was heated at 75° C. for 1 day. After being cooled to r.t., the reaction mixture was stirred with 1N KOH (0.5 mL) for 30 min and extracted with CH₂Cl₂. The organic layer was evaporated to afford IV, which was used for the next reaction without further purification. A THF (2 ml) solution of V containing Et₃N (45 mg, ca. 91 μmol) and the obtained IV was stirred over night, and the mixture was evaporated and purified by column chromatography (Sephadex; eluent, hexane:MeOH:CH₂Cl₂=2:1:1) to afford purple crystal VIII (19 mg, 23% based on 2,3,3-trimethyl-3H-indole). HRMS(ESI): [M+Na]⁺859.4465 (calc.859.4469); ¹H NMR (CDCl₃) δ1.16(s, 3H), 1.24(s, 3H), 1.39(s, 18H), 1.43(s, 18H), 2.80-2.95(m, 2H), 3.25(s, 4H), 3.30-3.40(m, 2H), 3.42(s, 4H), 3.58(d, J=6.3 Hz, 1H), 3.65(d, J=6.3 Hz, 1H), 5.96 (d, J=10.5 Hz, 1H), 6.61(d, J=7.5 Hz, 1H), 6.82(ddd, J=1.0, 7.5, 7.5 Hz, 1H), 6.88(d, J=10.5 Hz, 1H), 7.04(dd, J=1.0, 7.5 Hz, 1H), 7.13(ddd, J=1.0, 7.5, 7.5 Hz, 1H), 7.90(d, J=3.0 Hz, 1H), 8.26(d, J=3.0 Hz, 1H).

### 8,1′- BIPS-TA (IX)

To a CH₂Cl₂ (0.5 ml) of VIII (3.7 mg, 4.4 umol) was added trifluoroacetic acid (TFA) (2 ml) and the reaction mixture was stirred at r.t. over night. After evaporation of the solvent and TFA, the residue was subjected to column chromatography (Sephadex; eluent, hexane:MeOH: CH₂Cl₂=2:1:1) to afford IX (1.8 mg, 67%). ¹H NMR (CD₃OD) δ1.26(s, 3H), 1.30(s, 3H), 3.33(s, 4H), 3.52-3.62(m, 4H), 3.76(s, 2H), 3.82(s, 4H), 6.11 (d, J=10.4 Hz, 1H), 6.74(d, J=7.7 Hz, 1H), 6.90(dd, J=7.7, 7.7 Hz, 1H), 7.12(d, J=10.4 Hz, 1H), 7.13-7.20(m, 2H), 8.10(d, J=2.5 Hz, 1H), 8.20(d, J=2.5 Hz, 1H).

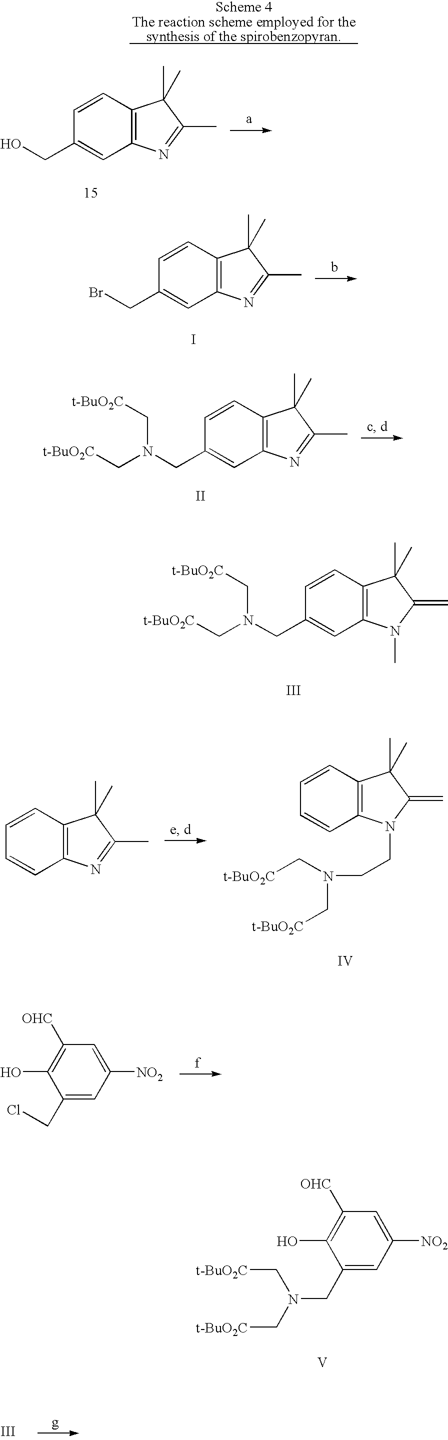

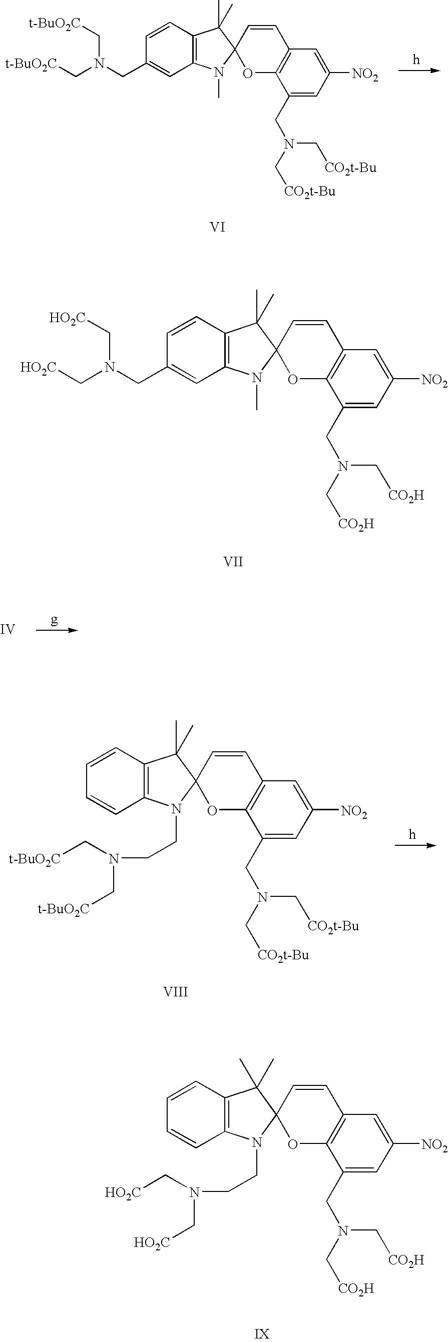

Reagents: (a) PPh₃,NMS, CH₂Cl₂; (b) Di-tert-butyl iminodiacetate, K₂CO₃, THF, reflux; (c) Mel, CHCl₃, reflux; (d) 0.5N NaOH (e) N,N-Bis[(tert-butoxycarbonyl)methyl]-2-bromoethylamine, CH₃CN; (f) Di-tert-butyl iminodiacetate; Et₃N, THF, reflux; (g) V, THF; (h) CF₃CO₂H, CH₂Cl₂.

Combinatorial Synthetic Approach

Indoline

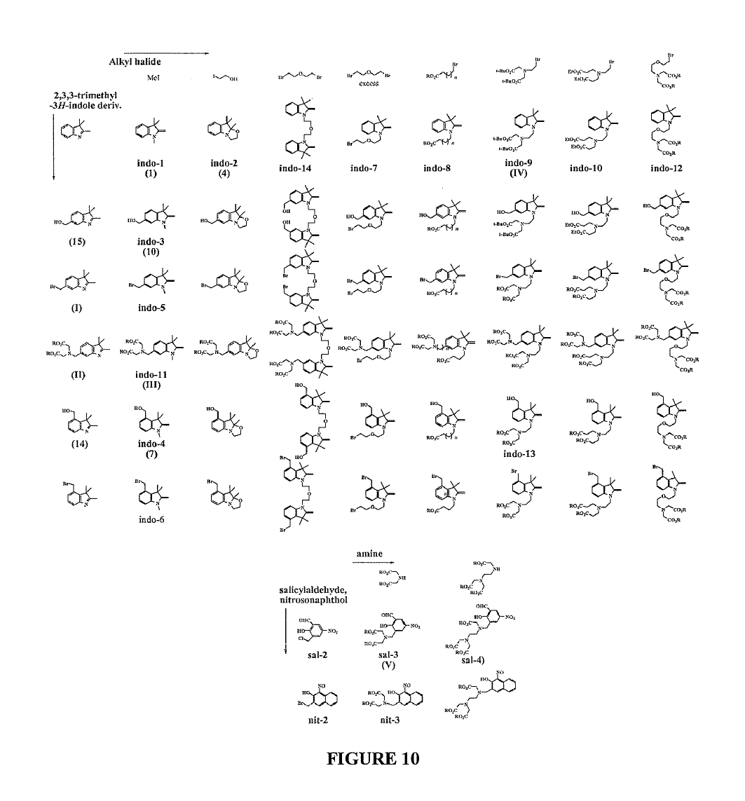

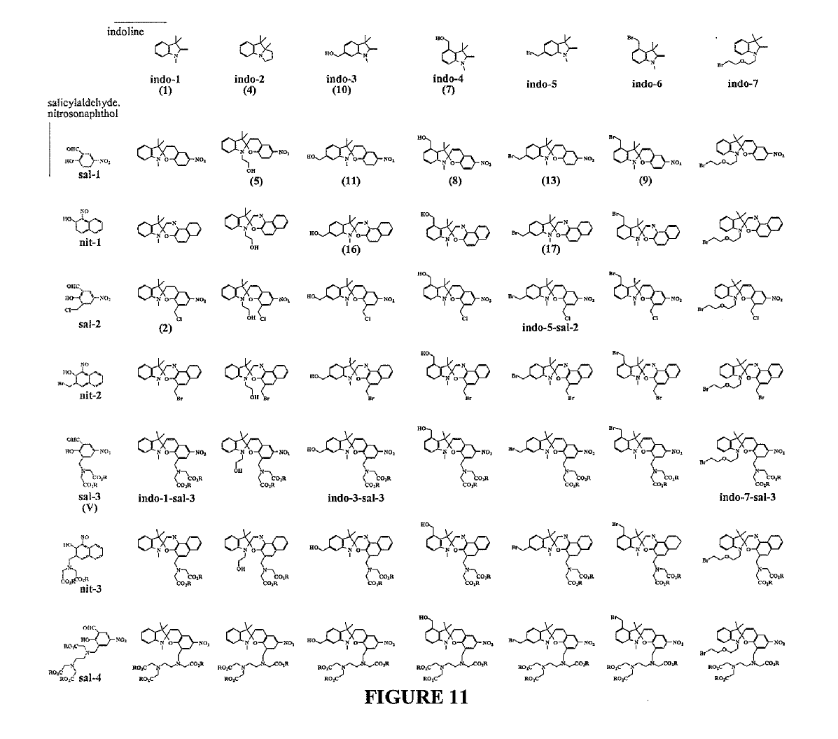

Indo-1 (1) is available from Sigma-Aldrich. All other indolines except for Indo-6, 12 and 13 were synthesized through the combinatorial approach (FIG. 10): coupling reaction of alkyl halide and 2,3,3-trimethyl-3H-indole. For example, Indo-5 was prepared from 1 and methyl iodide. Indo-7, Indo-8, Indo-10 and Indo-14 were synthesized from 2,3,3-trimethyl-3H-indole and the following alkyl halides respectively; 2-bromoethyl ether (excess), methyl 5-bromovalerate, N,N-bis(methyloxycarbonylethyl)-2-bromoethylamine, which is prepared from known compound 2-[N,N-bis(methyloxycarbonylethyl)amino]ethanol), and 2-bromoethyl ether (1 eq.). Detail of the synthesis of Indo-2 (4), Indo-3 (10), Indo-4 (7), Indo-9 (IV), and Indo-11 (III) are found in the section of thiol reactive and chelating probes.

General Synthetic Method

A solution (CH₂Cl₂ or CH₃CN) of indolenine and alkylhalide (excess amount) was refluxed for 12 h. After being cooled to r.t., the reaction mixture was stirred with 0.5 N NaOH (1 ml) for 15 min and extracted with CH₂Cl₂. The extract was evaporated to afford indoline as a crude product.

Additional Characteristic Data of Indolines:

Indo-5

¹H NMR (CDCl₃) δ1.53(s, 6H), 3.03(s, 3H), 3.85(s, 2H), 4.57(s, 2H), 6.43(d, J=7.8 Hz, 1H), 6.77(dd, J=7.8, 7.8 Hz, 1H), 7.12(d, J=7.8 Hz, 1H)

Indo-7

¹H NMR (CDCl₃) δ1.34(s, 6H), 3.41(t, 2H, 6.2), 3.71-3.74(m, 4H), 3.76(t, J=6.2 Hz, 2H), 3.86(d, J=1.6 Hz, 1H), 3.92(d, J=1.6 Hz, 1H), 6.65(d, J=7.5 Hz, 1H), 6.78(dd, J=7.5, 7.5 Hz, 1H), 7.09(d, J=7.5 Hz, 1H), 7.13(dd, J=7.5, 7.5 Hz, 1H)

Indo-8 (n=2)

¹H NMR(CDCl₃) δ1.34(s, 6H), 1.66-1.71(m, 4H), 2.34-2.39(m, 2H), 3.50-3.54(m, 2H), 3.67(s, 3H), 3.84(s, 1H), 3.87(s, 1H), 6.54(d, J=6.1 Hz, 1H), 6.76(dd, J=7.6, 7.6 Hz, 1H), 7.10(d, J=7.6 Hz, 1H), 7.12(d, J=7.6 Hz, 1H)

Indo-10

¹H NMR (CDCl₃) δ1.33(s, 6H), 2.47(t, J=7.2 Hz, 4H), 2.64(m, 2H), 2.85(t, J=7.2 Hz, 4H), 3.67(s, 6H), 3.58-3.70(m, 4H), 6.58(d, J=7.6 Hz, 1H), 6.77(dd, J=7.6, 7.6 Hz, 1H), 7.08(d, J=7.6 Hz, 1H), 7.12(d, J=7.6 Hz, 1H)

Indo-14

¹H NMR (CDCl₃) δ1.33(s, 12H), 3.63-3.68(m, 8H), 3.83(s, 1H), 3.87(s, 1H), 6.58(d, J=7.5 Hz, 1H), 6.79(dd, J=7.5, 7.5 Hz, 1H), 7.10(d, J=7.5 Hz, 1H), 7.11 (dd, J=7.5, 7.5 Hz, 1H)

Salicylaldehyde and Nitrosonaphthol

Sal-1, nit-1 and Sal-2 are commercially available from Sigma-Aldrich and TCl-America. The synthesis of sal-3 (V) is found in the section of chelating probes.

Spriobenzopyran and Spironaphthoxazine

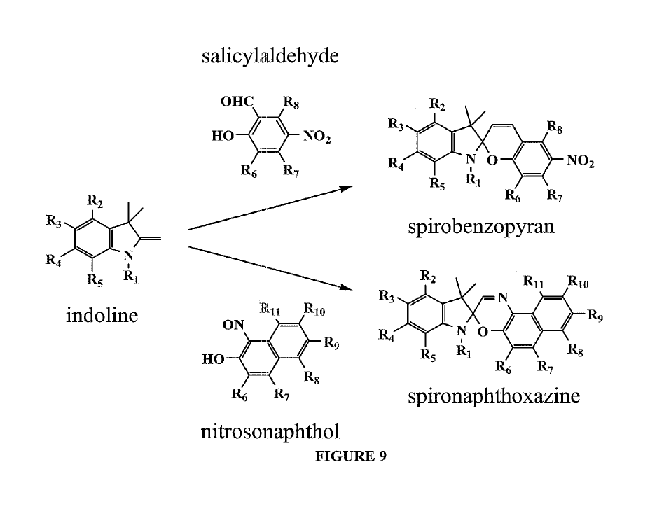

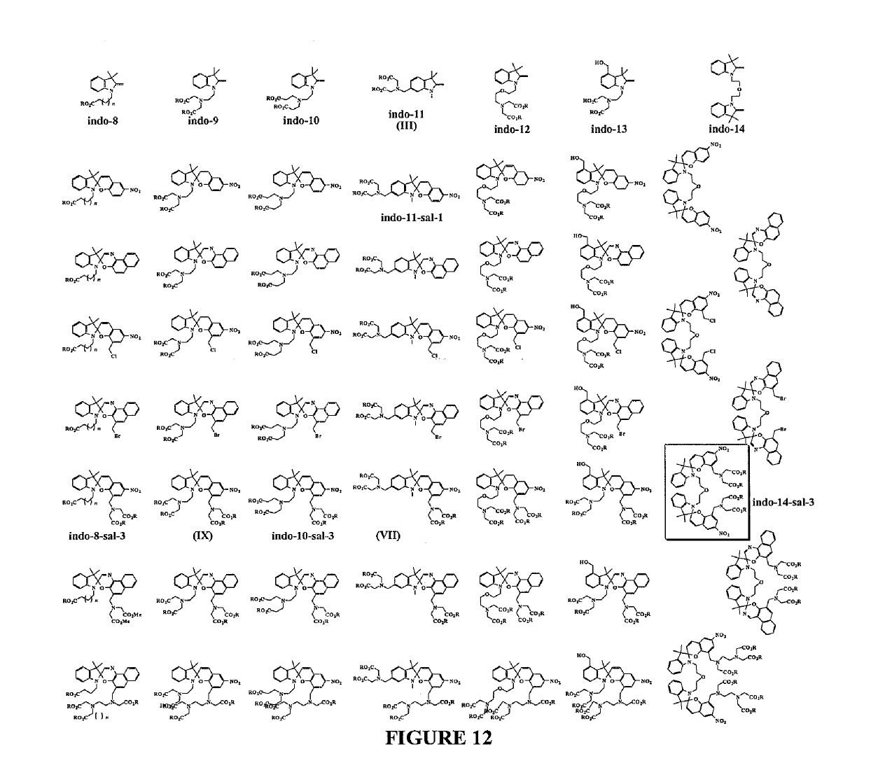

Spriobenzopyran and spironaphthoxazine are prepared via coupling reaction of indoline with salicylaldehyde or nitrosonaphthol (FIGS. 9, 11 and 12). Detail of the synthesis of Indo-1-sal-2 (2), Indo-2-sal-1 (5), Indo-3-sal-1 (11), Indo-3-nit-1 (16), Indo-4-sal-1 (8), Indo-9-sal-3 (R=tert-Bu, IX), and Indo-11-sal-3 (R=tert-Bu, VII) are provided in the section of thiol reactive and chelating probes. Indo-5-sal-1 (13), Indo-5-nit-1 (17) and Indo-6-sal-1 (9) were synthesized via bromination of Indo-3-sal-1 (11), Indo-3-nit-1 (16) and Indo-4-sal-1 (8) (see thiol reactive probe section), but it is likely that those compounds are available directly from the corresponding indolines.

General Synthetic Method

A THF or EtOH solution of indoline and salicylaldehyde (1.2 eq) or nitrosonaphthol (1.2 eq) is stirred at r.t. over night or at the refluxing condition for 6 h. After the evaporation of the reaction solvent, the residue was subjected to column chromatography (SiO₂ or Sephadex LH-20) to afford spirocompound.

Additional Characteristic Data of Spirocompounds:

Indo-1-sal-3 (R=Me)

MS(EI): 495(M⁺, 5), 422 (2), 84(100); HRMS(EI): M⁺495.1998 (calc.495.2006); ¹H NMR (CDCl₃) δ1.20(s, 3H), 1.27(s, 3H), 2.69(s, 3H), 3.27(s, 4H), 3.60(s, 3H), 3.59(d, J=14.2 Hz, 1H), 3.66(d, J=14.2 Hz, 1H), 5.87 (d, J=10.1 Hz, 1H), 6.54(d, J=7.6 Hz, 1H), 6.86(dd, J=7.6, 7.6 Hz, 1H), 6.92(d, J=10.1 Hz, 1H), 7.07(d, J=7.6 Hz, 1H), 7.17(dd, J=7.6, 7.6 Hz, 1H), 7.94(d, J=2.6 Hz, 1H), 8.12(d, J=2.6 Hz, 1H)

Indo-1-sal-3 (R=tert-Bu)

¹H NMR (CDCl₃) δ1.19(s, 3H), 1.27(s, 3H), 1.38(s, 18H), 2.70(s, 3H), 3.23(s, 4H), 3.61(s, 2H), 5.85 (d, J=10.1 Hz, 1H), 6.53(d, J=7.4 Hz, 1H), 6.84(ddd, J=1.2, 7.4, 7.4 Hz, 1H), 6.92(d, J=10.1 Hz, 1H), 7.06(dd, J=0.9, 7.4 Hz, 1H), 7.15(ddd, J=1.2, 7.4, 7.4 Hz, 1H), 7.92(d, J=2.6 Hz, 1H), 8.25(d, J=2.6 Hz, 1H)

Indo-3-sal-3 (R=Et)

HRMS(ESI): [M+H]⁺554.2515 (calc.554.2502); ¹H NMR (CDCl₃) δ1.21 (t, J=6.9 Hz, 6H), 1.22 (s, 3H), 1.27(s, 3H), 2.11 (t, J=6.0 Hz, 1H), 2.72(s, 3H), 3.27(s, 4H), 3.17 (d, J=17.5 Hz, 2H), 3.28 (d, J=17.5 Hz, 2H), 3.56 (d, J=14.4 Hz, 1H), 3.60 (d, J=14.4 Hz, 1H), 4.06 (q, J=6.9 Hz, 4H), 4.65 (d, J=6.0 Hz, 2H), 5.89 (d, J=10.2 Hz, 1H), 6.63 (d, J=1.1 Hz, 1H), 6.86 (dd, J=1.1, 7.4 Hz, 1H), 6.94 (d, J=10.2 Hz, 1H), 7.05 (d, J=7.4 Hz, 1H), 7.95 (d, J=2.6 Hz, 1H), 8.08 (d, J=2.6 Hz, 1H)

Indo-5-sal-2

¹H NMR (CDCl₃) δ1.21 (s, 3H), 1.31 (s, 3H), 2.71, (s, 3H), 4.34 (d, J=11.8 Hz, 1H), 4.40 (d, J=11.8 Hz, 1H), 4.48 (d, J=9.7 Hz, 1H), 4.52 (d, J=9.7 Hz, 1H), 5.59 (d, J=10.1 Hz, 1H), 6.54 (d, J=1.6 Hz, 1H), 6.92 (dd, J=1.6, 7.6 Hz, 1H), 6.96 (d, J=10.1 Hz, 1H), 6.99 (d, J=7.6 Hz, 1H), 8.01 (d, J=2.8, 1H), 8.15 (d, J=2.8 Hz, 1H)

Indo-7-sal-3 (R=tert-Bu)

HRMS(ESI): [M+H]⁺716.2513 (calc.716.2546); ¹H NMR (CDCl₃) δ1.19(s, 3H), 1.25(s, 3H), 1.39(s, 18H), 3.25(s, 4H), 3.27-3.81 (m, 6H), 3.43(t, J=6.0 Hz, 2H), 3.62(s, 2H), 5.95 (d, J=10.2 Hz, 1H), 6.61(d, J=7.5 Hz, 1H), 6.85(ddd, J=1.0, 7.5, 7.5 Hz, 1H), 6.91(d, J=10.2 Hz, 1H), 7.06(dd, J=1.0, 7.5 Hz, 1H), 7.15(ddd, J=1.0, 7.5, 7.5 Hz, 1H), 7.92(d, J=2.6 Hz, 1H), 8.25(d, J=2.6 Hz, 1H)

Indo-8-sal-3 (n=2, R=Me)

MS(EI): 595(M⁺,21), 522 (13), 86(100); HRMS(EI): M⁺595.2546 (calc.595.2530); ¹H NMR(CDCl₃) δ1.21(s, 3H), 1.27(s, 3H), 1.57-1.73(m, 4H), 2.31(t, J=6.7 Hz, 2H), 3.12(t, J=6.7 Hz, 2H), 3.30(s, 4H), 3.58(d, J=11.4 Hz, 1H), 3.62(s, 6H), 3.65(s, 3H), 3.66(d, J=11.4 Hz, 1H), 5.89 (d, J=10.2 Hz, 1H), 6.56(d, J=7.5 Hz, 1H), 6.86(dd, J=7.5, 7.5 Hz, 1H), 6.92(d, J=10.2 Hz, 1H), 7.08(d, J=7.5 Hz, 1H), 7.16(dd, J=7.5, 7.5 Hz, 1H), 7.94(d, J=2.8 Hz, 1H), 8.15(d, J=2.8 Hz, 1H)

Indo-10-sal-3 (R=Me)

HRMS(ESI): [M+H]⁺697.3057 (calc.697.3085); NMR (CDCl₃) δ1.17(s, 3H), 1.26(s, 3H), 2.37(t, J=7.2 Hz, 4H), 2.50-2.65(m, 2H), 2.74(t, J=7.2 Hz, 4H), 3.14-3.25(m, 2H), 3.31(s, 4H), 3.61(s, 6H), 3.62(s, 6H), 3.61-3.65(m, 2H), 5.88 (d, J=10.6 Hz, 1H), 6.57(d, J=7.6 Hz, 1H), 6.85(dd, J=7.6, 7.6 Hz, 1H), 6.93(d, J=10.6 Hz, 1H), 7.07(d, J=7.6 Hz, 1H), 7.16(dd, J=7.6, 7.6 Hz, 1H), 7.94(d, J=2.7 Hz, 1H), 8.16(d, J=2.7 Hz, 1H)

Indo-11-sal-1 (R=Et)

HRMS(ESI): [M+Na]⁺546.8217 (calc.546.2216); NMR (CDCl₃) δ1.19 (s, 3H), 1.27 (s, 3H), 1.29 (t, J=7.0 Hz, 6H), 2.75 (s, 3H), 3.61 (s, 4H), 3.92 (s, 3H), 4.19 (q, J=7.0 Hz, 4H), 5.86 (d, J=10.6 Hz, 1H), 6.67 (s, 1H), 6.79 (d, J=8.6 Hz, 1H), 6.84 (d, J=7.3 Hz, 1H), 6.93 (d, J=10.6 Hz, 1H), 7.01 (d, J=7.3 Hz, 1H), 8.01-8.05 (m, 2H)

Indo-14-sal-3 (R=tert-Bu)

HRMS(ESI): [M+Na]⁺1223.5823 (calc.1223.5892); NMR (CDCl₃) δ1.13(s, 3H), 1.14(s, 3H), 1.22(s, 3H), 1.23(s, 3H), 1.38(s, 36H), 3.20-3.60(m, 8H), 3.23(s, 8H), 3.58-3.60(s, 4H), 5.79 (d, J=10.3 Hz, 1H), 5.85 (d, J=10.3 Hz, 1H), 6.54(d, J=6.9 Hz, 1H), 6.57(d, J=6.9 Hz, 1H), 6.77(d, J=10.4 Hz, 1H), 6.84(d, J=10.4 Hz, 1H), 6.82-6.88(m, 2H), 7.05(d, J=7.4 Hz, 2H), 7.09(dd, J=7.4 Hz, 2H), 7.12(dd, J=7.4, 7.4 Hz, 1H, 7.88(m, 2H), 8.24(m, 2H)

### EXAMPLE II

### Labeling of Proteins with Thiol Reactive Spirobenzopyrans

Rabbit muscle G-actin was purified according to Marriott¹⁴ in G-buffer (2 mM Tris, 0.2 mM CaCl₂, and 0.2 mM ATP at pH 8.0). The concentration of G-actin was determined by absorption spectroscopy using an extinction coefficient of 3400 M⁻¹ cm⁻¹ at 290 nm¹⁴. 1 ml of a 20 μM solution of G-actin was treated with 20 μL of a 10 mM DMF stock solution of each thiol reactive, spirobenzopyran probe [3, 6, 9, 12, 13]. The reaction mixture was left in the dark for 2 hours at room temperature. The protein was centrifuged for 10 min at 2000 g at 4° and applied to a Bio-Rad PD-10 column equilibrated in G-buffer containing 1 mM DTT. If necessary the conjugate was dialyzed against 1 L of G-buffer at 4°. The labeled G-actin solution was clarified by centrifugation (100000 g for 1 hour) before absorption spectrometry. The extinction coefficient for the SP probe is taken as 35,000 M⁻¹cm⁻¹ for the near ultraviolet absorption maximum and 52,000 M⁻¹cm⁻¹ for the maximum visible wavelength for the MC probe¹⁵.

RESULTS AND DISCUSSION for Example I and II

The goal of the synthetic work was to prepare a family of optical switches for the specific labeling of biomolecules. The reactive groups (bromo-, iodo- and maleimido), which were introduced to different sites on the spirobenzopyran ring, were used to attach a common photochrome to thiol groups within biomolecules. This family of photochromic reagents allows control of the chromophore dipole geometry within the bioconjugate. This represents a new development in bioconjugate chemistry that will be useful for spectroscopic studies of biomolecular structure and dynamics.

Synthesis of Thiol Reactive Spiropyrans.

The synthetic approach used to prepare the thiol reactive optical switches is summarized in schemes 1 and 2. The syntheses involve coupling the indoline derivatives (1, 4, 7, 10) with the corresponding salycilaldehydes to yield the four key spirobenzopyrans (2, 5, 8, 11)¹⁶⁻¹⁸. The thiol reactive spirobenzopyrans (3, 6, 9, 12, 13) are prepared from the corresponding spirobenzopyrans via the halogen exchange reaction, bromination of alcohol or modified Mitsunobu reaction¹⁹.

Optical Spectroscopy of the Spirobenzopyran Chromophore in Solvents

The characterization of the effects of specific solvent interactions on the MC absorption spectrum will be important to interpret the nature of MC interactions within a protein. The average energy of the lowest energy absorption band for MC contains information on the dielectric constant of the solvent and the presence of specific solvent effects, such as H-bonds and dipole-dipole interactions. The ground and excited state dipole moment for polar aromatic probes such as MC are often defined by the nature and location of polar groups within the conjugated ring system²⁰. In the case of MC, the dipole will most likely be defined by the positive nitrogen atom and the negative nitro-group—these two groups may be considered as monopoles since they are well-separated by the aromatic rings and olefin (FIG. 1A). The permanent charge on the positive monopole and highly polarized oxygen on the negative monopole are most likely responsible for the fact that the dipole moment of MC is higher in the ground state (20 D) than the excited state (14 D)²¹.

Absorption and fluorescence spectroscopy can provide important information on the nature of molecular interactions between the SP and MC states and their molecular environment^{15,22}. For example, the similarity in the MC absorption spectra of the five spirobenzopyrans (FIG. 1A; 3, 6, 9, 12, 13) dissolved in ethanol suggests that alkyl substitution on the MC does not change its aromaticity nor the interactions of the MC probe with solvent molecules, although N-alkylation does slightly red-shift the absorption spectrum (FIG. 2A). Therefore, any solvent-induced shift in the absorption spectrum of MC must result from differences in the dielectric constant and specific MC-solvent interactions. Support for this hypothesis is provided in a study on the dependence of the absorption spectrum of MC (compound 12) on the solvent (water, 2-propanol, 1,2-propanediol, acetonitrile and dichloromethane; FIG. 2B). The absorption spectrum of the MC chromophore is shown to be sensitive to the nature of the solvent; blue-shifts are observed within polar solvents while apolar solvents elicit red-shifts. Further analysis of these data reveals that these spectral shifts are associated with specific MC-solvent interactions. For example, the absorption spectrum of MC in 1,2-propanediol is blue-shifted compared to in 2-propanol even though their dielectric constants are reasonably similar (32 and 20.1 respectively)—the inventors believe that this blue-shift results from the greater H-bonding capacity of 1,2-propanediol compared to 2-propanol. The MC spectrum is even further blue-shifted in water which, the inventors believe, reflects the stronger hydrogen bonds and higher dielectric medium. The most red-shifted absorption spectra are found in apolar, low dielectric constant solvents, such as dichoromethane. Interestingly, Gorner¹⁵ has shown that the quantum yield for the SP-MC transition also depends on the solvent polarity and ranges from 0.75 for toluene to 0.1 for acetonitrile. Together these spectral studies show that the average energy of the MC absorption spectrum is sensitive to the changes in the dielectric constant of the solvent and to specific solvent dipolar interactions.

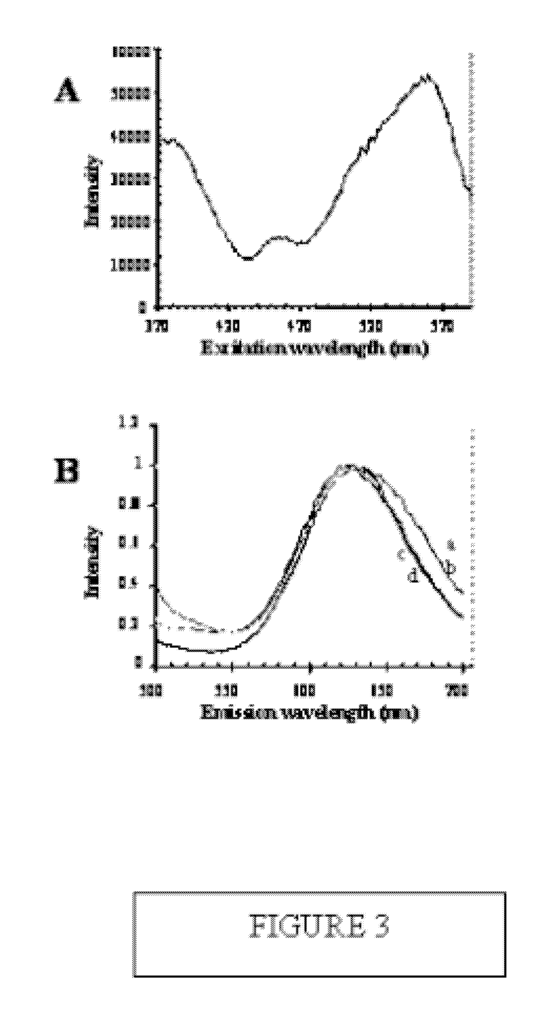

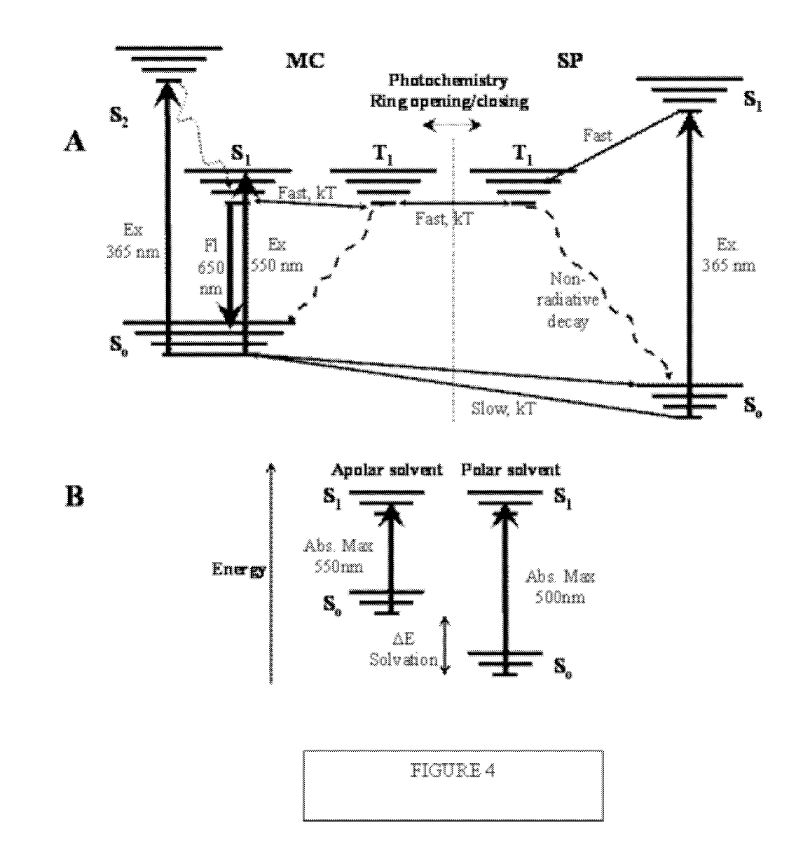

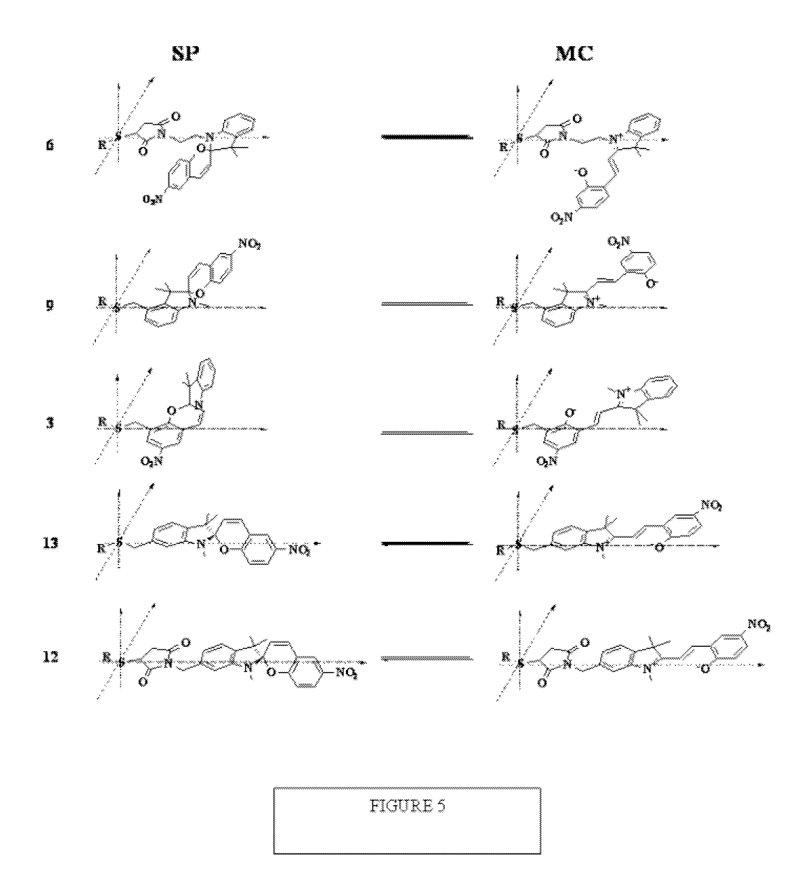

Further information on MC-solvent interactions can be derived from studying the fluorescence properties of MC. From a practical standpoint, fluorescence from ¹MC* is best studied by exciting the spirobenzopyran with mid-ultraviolet light (S₀—S₂ transition)—this condition maintains a uniform population of MC excited state molecules (see Jablonski diagram, FIG. 4A). Contrary to the ground state studies, the inventors find that the average energy of the MC fluorescence is insensitive to the solvent (water, 2-propanol, 1,2-propanediol and acetonitrile; data not shown) and to the location of MC probe on G-actin (FIG. 3B).

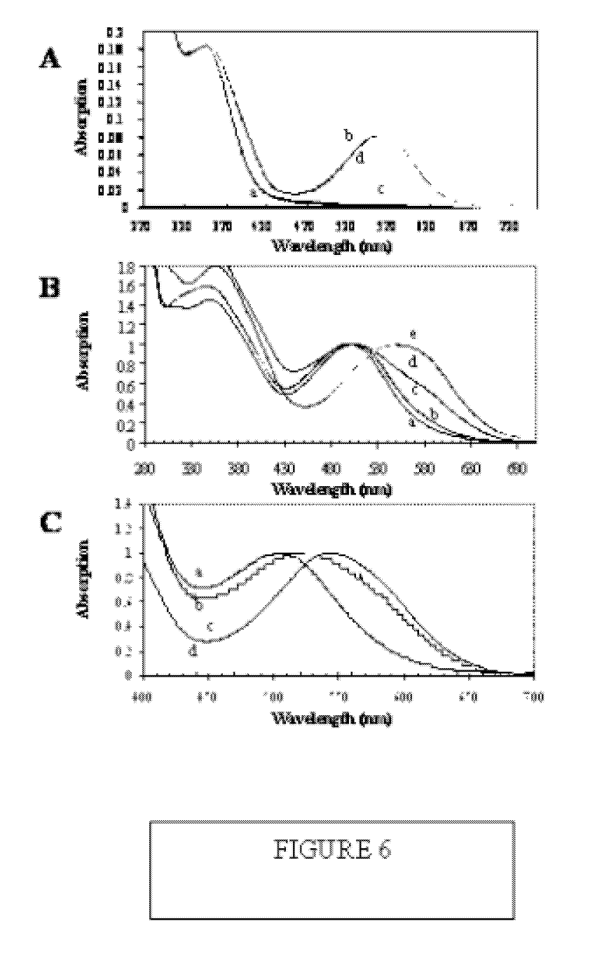

An explanation for the anomalous polar solvent-induced blue-shifts in the MC absorption spectrum and the insensitivity of MC fluorescence to polar solvents is provided by considering an unusual property of the MC dipole moment—Bletz et al have shown that the MC dipole is lower in the excited state (14 D) compared to the ground state (20 D)²¹. Dipolar interactions between the MC dipole and polar, H-bonding groups (e.g. water and the peptide bond in proteins) should therefore be stronger in the ground state compared to the excited state. Furthermore the difference in the interaction energy for specific dipolar interactions between solvent molecules and the MC dipole will be greater in the ground state compared to the excited state as the inventors show in the absorption and fluorescence studies (e.g. FIGS. 2B, 5B, C and 3B).

Although the observed fluorescence of MC is weak (FIG. 3A), primarily because the quantum yield for the photochemical conversion to SP is already high¹⁵, the emission will nonetheless prove to be useful in understanding the photophysics of MC as well as providing a sensitive signal to quantify MC levels in complex samples e.g. within a cell.

Effect of Linkage Geometry on the Absorption Spectrum of Spirobenzopyran Conjugates

Having shown that the MC absorption spectrum is sensitive to specific dipolar interactions related studies were performed to characterize the dipolar environment around MC probes specifically attached to biomolecules.

G-Actin: The reaction between a ten-fold molar excess of thiol reactive, spirobenzopyran reagents and 20 μM G-actin at 20° is complete within 2 hours. The concentration of spirobenzopyran in each conjugate is determined from the value of the absorption maximum of the SP form of G-actin conjugates using an extinction coefficient of 35,000 M⁻¹cm⁻¹ at 350 nm¹⁵ (FIG. 5A) The absorption spectra of the MC and SP states are not affected by the labeling ratio (spirobenzopyran/G-actin, which varies from 0.6 to 0.8 within the different conjugates. Cysteine-374 is located at an important site on G-actin and has been implicated in several important interactions of actin that include protomer-contacts within F-actin²³, the binding of the actin filament capping proteins Gelsolin and CapG²⁴ and the motor protein, myosin II²⁵. The purpose of this study is to show that an analysis of the absorption properties of spirobenzpyran differentially linked to G-actin can provide important information on specific dipolar interactions between MC and G-actin around the probe attachment site. Irradiation of the five spirobenzopyran conjugates with 365 nm light for 30 seconds generates the highly colored MC probe (curve b in FIG. 5A). On the other hand, excitation of these MC conjugates with 546 nm light for 30 seconds results in a photochemical transition back to the colorless SP state (curve c in FIG. 5A). Optical control of the MC and SP probes on G-actin is efficient and reversible and this cycle of UV and Visible excitation can be repeated over many cycles (curve d in FIG. 5A). The MC absorption spectrum on G-actin is very similar over optical switching cycles and since the MC absorption spectrum is highly sensitive to the molecular environment the inventors therefore believe that optical switching between SP and MC faithfully reproduces the specific dipolar interactions between the MC probe and the protein. The inventors note however that the slight difference in the UV region of the MC-G-actin spectrum (FIG. 5A) that a small population of MC probes may undergo secondary photochemistry e.g. bleaching as is found for most organic probes.

Given the sensitivity of the MC absorption spectrum to specific solvent interactions, it is not surprising the average energy and shape of the lowest energy MC-G-actin absorption transition is also dependent on the MC-cysteine-374 linkage geometry (FIG. 5B). This arises because the different locations of the thiol reactive groups position the MC probes at unique sites on G-actin. The MC probes will therefore engage in specific interactions with apolar and polar groups in their vicinity that may include weak Van der Waals contacts and stronger dipolar interactions between MC and peptide bonds, polar amino acid side groups, surface water and charged amino acid residues²⁶. The absorption studies show that the average energy of the MC absorption spectrum on G-actin varies from 20,000 cm⁻¹ (500 nm) for compound 9, to 18,132 cm⁻¹ (551.5 nm) for compound 12, which corresponds to a free energy difference of over 1,868 cm⁻¹ or nearly 10 kT. Interestingly, the difference in the average absorption energy between compounds 12 and 13 on G-actin is also large (1400 cm⁻¹), in spite of the fact that the thiol reactive group is situated on an identical atom—this result suggests that the further displacement of MC in compound 12 on G-actin afforded by the maleimido linker, positions the MC dipole in 12 to a site on G-actin where it engages in stronger dipolar interactions compared to compound 13.

The near ultraviolet SP absorption spectra in the five spirobenzopyran G-actin conjugates differ to a lesser extent than that found for MC, which the inventors believe is a result of the lower SP ground state dipole moment at 5 D compared to that of MC at 20 D²¹.

BSA: BSA conjugates of the spirobenzopyran reagents were prepared in a similar fashion to that described for G-actin yielding similar labeling ratios (FIG. 5C). The absorption spectrum of the five MC probes attached to a cysteine residue on BSA are centered at 544.5 nm (18,365 cm⁻¹) for compound 6,534 nm (19,861 cm⁻¹) for compound 12, 520 nm (19,231 cm⁻¹) for compounds 9; 503.5 nm (19,861 cm⁻¹) for compound 13. The difference in the energy of the absorption bands for these conjugates of 1496 cm⁻¹ is a little less than that found between the same probes on G-actin but again is significant in terms of the free energy of complex formation between BSA and its ligands. These confirm the conclusion that the protein matrix is made up of a complex dipolar landscape.

Optical Switching of Dipolar Interactions within Spirobenzopyran Conjugates:

The inventors propose a model to understand the origin of different interactions of MC within a protein that is based on a qualitative comparison of the different linkage geometries between the five spirobenzopyran probes and the thiol group on the protein. As shown in FIG. 6, the different linkages force the MC and SP probes to adopt different locations in the conjugate where they engage in different interactions. The inventors hypothesize that the strength of the MC interaction with the protein is considerably stronger compared to the SP state because of the difference in the dipole moments of the MC ground state (20 D) versus the SP ground state (5 D). The orientations of the MC and SP dipoles schematized in FIG. 6 are derived on the assumptions that: (1), the sulphur atom on the biomolecule is located at the origin while the position of the spirobenzopyran is constrained by forcing the atom containing the thiol group to lie on the x-axis; (2), the structures of the SP and MC molecules are the same as those derived from crystallographic studies. Under these conditions, the direction of the MC dipole will be reversed in conjugates of compounds 6 and 9, and will survey a broad range of orientations in the other conjugates (3, 12 and 13). This model is supported by the findings of the MC solvent and protein absorption studies showing that the protein matrix has a diverse dipolar landscape that can vary widely from polar to apolar over a very narrow volume element.

The results from the MC-protein absorption studies suggest that dipolar interactions between MC and the protein are specific and site selective—the free energy (difference) measured for MC probes attached to a unique cysteine residue with different linkage geometries is similar to that found in complexes of actin with actin binding proteins and other ligands. However, the interaction between the MC probe and the protein can be significantly reduced by optically converting MC to SP, which will assume a different location and engage in weaker interactions with the protein. The challenge for ongoing studies is to prepare spirobenzopyran protein conjugates in which the MC or the SP state competes effectively on the protein for the binding site of a regulatory ligand, as depicted in FIG. 1 B.

Thermally-Driven Transitions Between MC and SP

The rate for the thermal equilibration of MC to SP in ethanol was measured using the decrease in the maximum MC absorption value as a function of time in the dark (FIGS. 7A, B). These data are fit using a single exponential function and yield a decay rate of 0.0037 sec⁻¹. This rate, however, is too fast for many of the envisioned applications of optical switches in biology. To determine the effect of protein interactions on the thermally driven rate between MC and SP the inventors conducted a related kinetic study for the G-actin and BSA conjugates of the five spirobenzopyran. In almost all cases the MC absorption either unchanged or slightly decreases in these conjugates during the 60 minute measurement period (the example shown in FIG. 7C is the G-actin conjugate of compound 9). This result suggests that the MC—SP transition is hindered in these G-actin conjugates most likely as a result of strong dipolar interactions of the MC probe within the protein. The inventors propose that these interactions impose an energy barrier for the MC—SP transition. The inventors conclude, therefore that transitions between the SP and MC states of spirobenzopyran in protein conjugates can be selectively controlled by irradiation with near ultraviolet and visible light. Furthermore since photochemical reactions responsible for the SP and MC transitions occur in the excited triplet states of SP and MC¹⁵, the inventors expect that optical switching between the SP and MC states in spirobenzopyran conjugates with concomitant modulation of functional interactions or activity will occur on a timescale of 1-10 microseconds.

Highlighting Applications of Photochromic Probes in FRET:

The ability to differentially project an acceptor probe with a well-defined dipole moment to different locations on a protein can be used as part of a new approach to improve the precision of proximity determinations using FRET; First, the ability to shift the absorption maximum of a common MC chromophore using different probe-protein linkage geometries can be used to change the overlap integral between the donor and acceptor probes and to “tune” the Foerster distance between an identical pair of donor and acceptor probes²⁸. To illustrate this potential the inventors used Foerster theory²⁷ to highlight the effects of MC absorption spectral shifts in G-actin conjugates of compounds 9 and 13 (maximum absorption of 500 nm and 551.5 nm respectively; MC ∈ₘₐₓ=52,000 M⁻¹cm⁻¹; K²−⅔; n=1.4, φ=0.7) on the overlap integral (J) with a simulated tetramethylrhodamine (TMR) donor spectrum fluorescence maximum at 575 nm)—j increases from 1.10×10¹⁵ M⁻¹·cm⁻¹·nm⁴ in compound 9 to 4.21×10¹⁵ M⁻¹·cm⁻¹·nm⁴ in compound 13, which would lead to a tuning of the Rₒ in this simulated TMR-MC-acceptor pair from 4.8 nm to 6.0 nm for compounds 9 and 13 respectively. Clearly by choosing other MC-conjugates of G-actin shown in FIG. 5B it will be possible to tune the Rₒ between these two distances. Second, by controlling the linkage geometry between an identical acceptor chromophore and the protein (FIG. 6) it should be possible to generate a family of spirobenzopyran conjugates for which the average orientation of the MC dipole is quasi-random. Global analysis of FRET data between an identical donor and the five MC probes labeled at a unique site in a complex can be used to satisfy the commonly employed assumption that the orientation factor (k² value) between a donor and acceptor pair can be taken as ⅔—the adoption of this new experimental approach should reduce the uncertainty associated with this assumption.

Summary for Examples I and II

A family of thiol reactive spirobenzopyran reagents is described that can be specifically attached to biomolecules where they undergo efficient and reversible, light-directed structural transitions between two states (SP and MC) that have widely different structural and physical properties. Differences in the properties and interactions between SP and MC within a bioconjugate are being exploited as part of a new approach to achieve reversible, optical switching of biomolecular interactions. The inventors show that the strength and nature of the dipolar interactions between MC and the biomolecule depend on the MC linkage geometry and that these interactions prevent the thermally-driven transition between MC and SP. The difference in the free energy of interactions between MC probes and G-actin is comparable to that found for complexes of G-actin with regulatory proteins (˜6 kcal/mol)—together the studies described in this work demonstrate the feasibility of using site-selectively labeled photochromes as optical switches for the reversible, modulation of biomolecular interactions e.g., protein-protein, protein-Ca²⁺. The inventors also highlight how the family of spirobenzopyran reagents described in this work can be used to tune the Foerster distance and to provide an experimental system to evaluate the K² value in FRET based analysis of molecular complexes.

### Example III

### Calcium Estimation and Binding

The concentration of intracellular Ca²⁺ rapidly increases during the activation of membrane-activated cell signaling pathways that leads to motility, muscle contraction and exocytosis. The change in [Ca²⁺] is modest (2˜5-fold) and is usually only effective close to the plasma membrane unless intracellular Ca²⁺ stores are released. Ca²⁺-binding to signaling proteins is an early step in the regulation of cellular processes and usually leads to structural changes in the protein that trigger a cascade of functional protein interactions and activities that ultimately lead to a physiological response. Understanding the mechanisms underlying calcium ion signaling therefore requires techniques capable of mapping and manipulating [Ca²⁺] within the cell and performing correlative molecular analysis to show how local changes in [Ca²⁺] are coupled to the activation of Ca²⁺-binding signaling proteins and pathways and to the global physiological response. The Tsien laboratory revolutionized the field of Ca²⁺ signaling through the development and application of optical probes for imaging or perturbing Ca²⁺ (Tsien, 1989)—these probes include fluorescent Ca²⁺-indicators such as Indo-1 that bind to Ca²⁺ in a rapid and reversible reaction and are effective in interrogating intracellular [Ca²⁺] over many excitation cycles at non-buffering levels, and photomodulatable Ca²⁺-chelators that release or sequester Ca²⁺ in a light-driven reaction.

Research aimed at understanding the mechanisms that underlie Ca²⁺-triggered physiological processes such exocytosis and muscle contraction, which occur on a microsecond timescale, can only be realized using a chemical relaxation approach. Temperature, pressure and flow perturbation techniques are either unsuitable or non-specific in changing intracellular [Ca²⁺] and so considerable effort has been expended in developing photoactivatable Ca²⁺-chelators, such as Nitr-5 and NP-EGTA, to generate Ca²⁺ transients at defined sites with cells. However, these probes can usually only be used to generate a single perturbation of Ca²⁺ because the photoisomerization reaction, which occurs with a rate of 10⁴ s⁻¹ at best, is irreversible and generates photoproducts that are either reactive or maintain an affinity for Ca²⁺ (Nitr-5). These properties not only limit the usefulness of the caged Ca²⁺ probe but their effects must be evaluated in separate, control experiments.

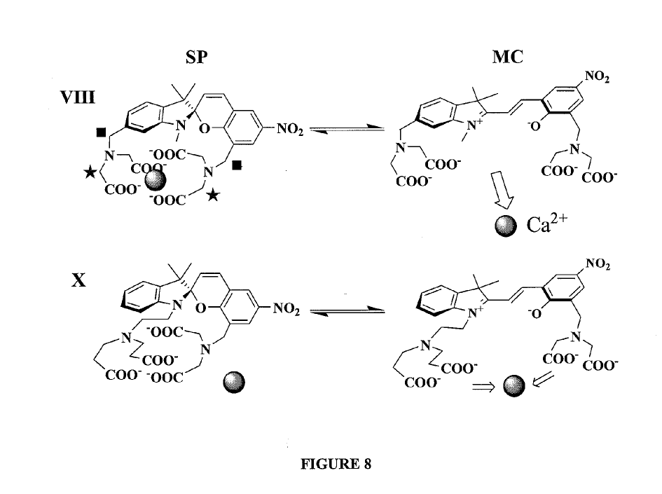

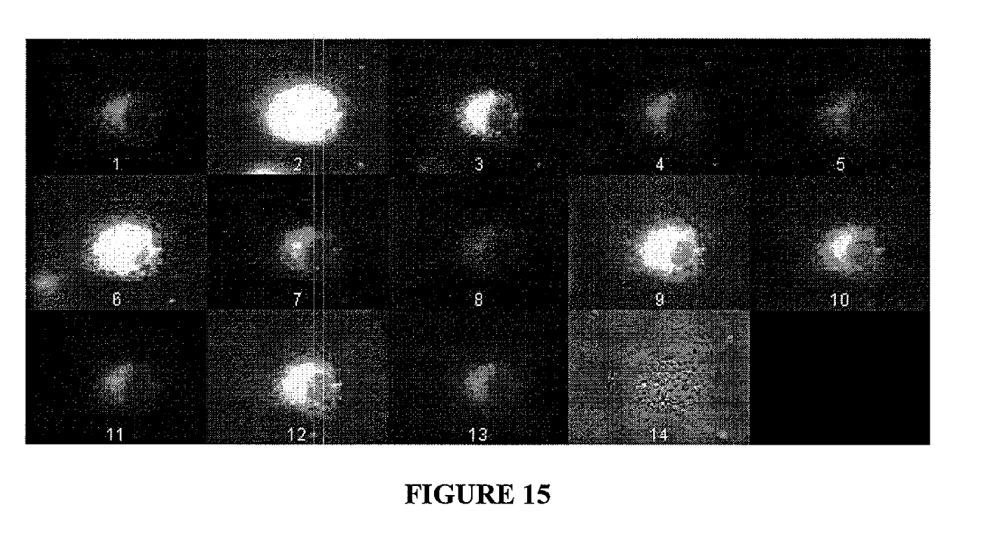

The inventors believe that the ideal Ca²⁺-perturbation probe would incorporate both Ca²⁺ sequestering and release activities that are controlled through reversible, efficient and rapid, light-driven reactions without the release of secondary products. As part of a new program to develop such probes, the inventors introduce a new approach in the design of optical probes capable of generating specific and reversible, light-directed perturbations of Ca²⁺ and Ca²⁺-binding proteins within cells. The approach is based on interesting properties of benzospiropyrans, which undergo rapid and reversible transitions between a colorless spiro-(SP) state and a colorful merocyanine (MC) state (Inouye, 1994). Transitions between the two states of the switch can be controlled by exciting the SP state with 365 nm light (SP—MC) and the MC state with 546 nm light (MC—SP). A family of optical switches was designed and synthesized with the aim of positioning the four carboxyl groups on the optical switch such that they exhibit a high affinity for Ca²⁺ in either the SP or MC state. Since the two aromatic rings in the benzospiropyran are orthogonal the inventors reasoned that this state would not bind Ca²⁺ very tightly whereas the positioning of the four carboxyl groups in the planar MC state would lead to strong Ca²⁺ chelator (FIG. 8). The different synthetic approaches used in these studies are summarized in the materials and methods section. FIG. 8 also illustrates optical switching of calcium using two optical switch chelates—the two probes in this figure have been synthesized and chemically characterized. In addition the inventors describe an approach to synthesize numerous new chelates using chemical combinatorial chemistry shown in the FIGS. 10, 11, 12 and 15—specific calcium probes are shown in FIG. 15. Introduction of optical switches incorporating both a SBP and a SNZ switch may be used to develop switches incorporating >2 way control (off-ON)—thus by independently triggering the MC to SP transitions in the SBP and the SNZ is should be possible to make a 4 way switch (SP/SP, SP/MC, MC/SP and MC/MC)—these may correspond to ON and OFF and standby or to create variable control (OFF, ⅓ on, ⅔ ON and FULL ON).

The dissociation constant for the SP—Ca²⁺ complex of ˜500 μM for compound X shown in FIG. 8—this was determined in a competition assay using the red-shifted fluorescent indicator Ca²⁺ indicator, Rhod-2.

Irradiation of the SP calcium switch (Compound X in FIG. 8) loaded into Hela cells with 365 nm light for 0.5 seconds resulted in the quantitative generation of the MC state as seen in the fluorescence emission of the cell—continued irradiation of these cells with 546 nm light stimulates the fluorescence and the conversion of MC back to the non-fluorescent SP state—repeat cycles of UV and VIS excitation of this cell show the ability to rapidly and reversibly manipulate the calcium binding properties of compound X. (See FIG. 15) Similarly, compound VIII and the bis-SBP chelate highlighted in the combinatorial chemistry figure above, will also exhibit affinities for calcium in either the SP or MC state that are more suitable for cell applications. Nonetheless, compound X may be used to manipulate calcium and other divalent metal ions with light—applications are envisioned for chips that can be recycled, using light, for the analysis of metal ions in solution or air.

An attractive feature of the Ca²⁺ switches described in this invention is the very fast rate of optical switching between the strong and weakly binding Ca²⁺ states. Studies by Horner (2001) suggest that the photoisomerization of benzospiropyrans occurs via triplet state reactions within 11 μs. If Ca²⁺ is released from the MC state on a similar timescale then this class of probe will lead to an improvement in the rate of Ca²⁺-perturbations by at least 3 orders of magnitude (Adams and Tsien, 1989).

A complicating factor in the use of benzospiropyrans is that the MC—SP reaction may also occur in the ground state driven by thermal fluctuations (Sakata et al, 2004). This property may lead to the leaching of Ca²⁺ from MC—however, the inventors note this effect will have limited impact in most applications since the rate for the thermally driven process is slow (0.02 s¹⁻) at 37 c compared to the excited state (10⁵ s⁻¹). Furthermore the ground state reaction provides an additional degree of control of the Ca²⁺-switch. The inventors also note that a further reduction in the rate of the thermally driven MC—SP reaction can be realized by attaching the optical switch to proteins (Sakata et al, 2004). Also, the rate for the MC to SP transition for SNZ is 1000 times faster than SBP.

The family of Ca²⁺-optical switch was introduced into living cells as methyl esters. The efficiency of the loading was determined by fluorescence imaging of the MC state using and excitation wavelength of 546 nm and emission of >600 nm. Since excitation of MC leads to the MC—SP transition and loss of fluorescence this analysis should be conducted using a single pulse of 546 nm light. In most cell studied the optical switch entered the cell within a few minutes and while initially it was localized within vesicles within 30 minutes the fluorescence was generally uniformly distributed within the cell. The efficiency of the de-esterification of the carboxyl groups was not determined but assumed, on the basis of functional Ca²⁺ responses, to be complete within the incubation period.

The family of Ca²⁺-optical switch described in this invention has built-in design features that can be used to tune the maximum of the action spectrum for the MC—SP transition. The nitro group generates a weakly fluorescent MC state, which can be used to quantify the kinetics of Ca²⁺-perturbations and to image the distribution of the MC state within living cells. Interestingly the maximum MC absorption spectrum of switches lacking the nitro group is shifted to 620 nm and leads to a non-fluorescent MC state. However, this class of photochrome exhibits a remarkable degree of photostability that allows fully reversible optical switching between the SP and MC states over numerous irradiation cycles.

A second important design feature is the incorporation of reactive functional groups onto the optical switch, which can be used to prepare conjugates of proteins and other biomolecules as well as surfaces containing amino or thiol groups. These conjugates can be used to target the optical switch to specific sites in the cell e.g. membrane, actin cytoskeleton, or to restrict the optical switch to specific sites on a chip or surface for applications in biotechnology.

### CONCLUSION FOR EXAMPLE III

The inventors have introduced a new class of calcium ion chelating probe that undergo rapid and reversible, light-directed transitions between two structural states that exhibit widely different affinities for calcium ions. The advantages of this approach to perturbing [Ca²⁺] compared to the caged Ca²⁺ approach include: 1), a single probe that can be used to sequester or release Ca²⁺ using light; 2), the transitions are fully reversible and can occur exclusively in the excited state or a combination of an excited state and a thermally driven ground state reaction; 3), the transitions are rapid (11 μs) and proceed with almost perfect quantum efficiency; 4), the transitions do not involve the release of photoproducts and are therefore free of artifacts associated with 2-nitrophenyl based caged groups; 5), the action spectrum for the MC—SP transition can be tuned over a broad wavelength range (500 nm-750 nm) to limit interference from other optical probes in the sample.

Kinetic mapping of Ca²⁺-mediated signaling pathways also requires analysis of downstream protein targets. Some years ago the inventors introduced a technique to generate spatially and temporally defined perturbations of specific proteins using light-directed of caged proteins. The caged protein approach is also affected, but to a lesser extent, by the limitations outlined for caged Ca²⁺ chelators. The inventors will use this approach to generate perturbations of Ca²⁺-binding proteins, troponin C, calmodulin and CapG, which are known to underlie the regulation of muscle contraction and cell motility respectively. The inventors will also show how a new class of optical probe can be used to generate conjugates of cTnC, calmodulin and CapG whose Ca²⁺-binding properties can be rapidly and reversibly modulated using light-directed.

Combinatorial Synthesis of Calcium Chelates.

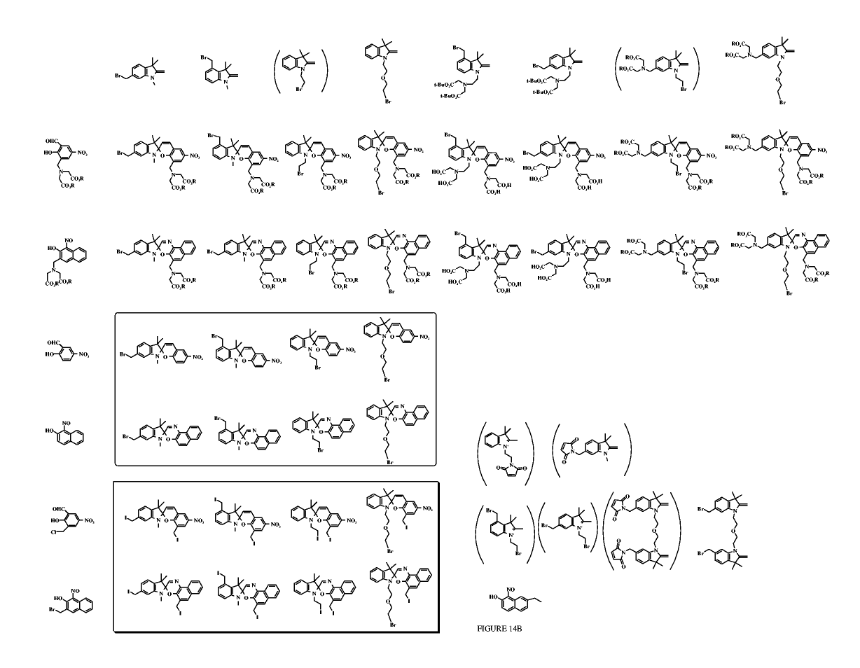

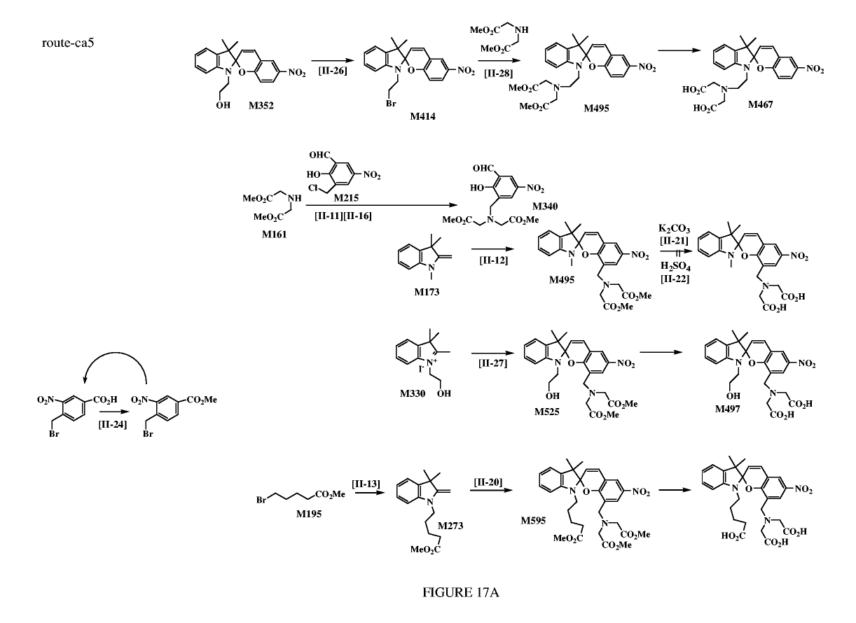

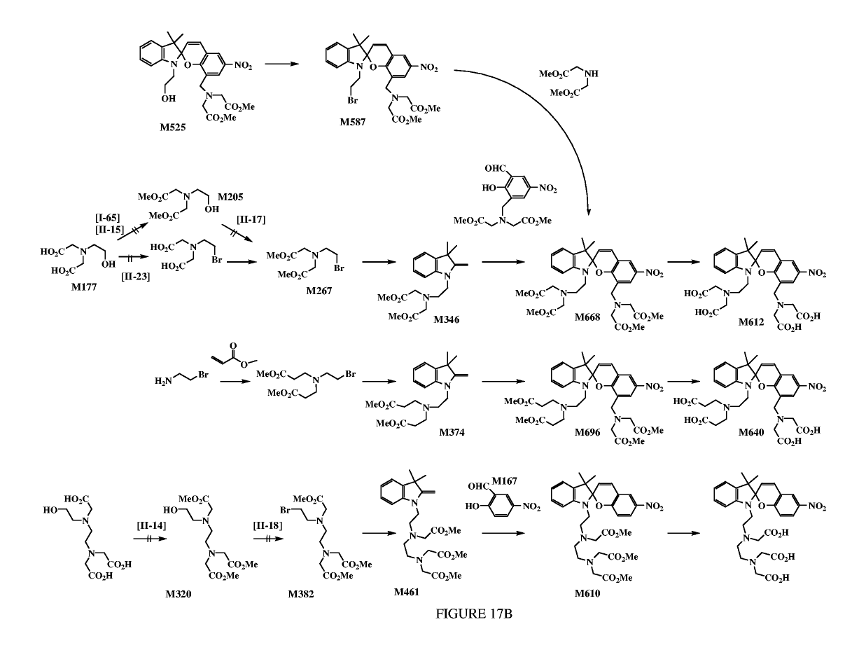

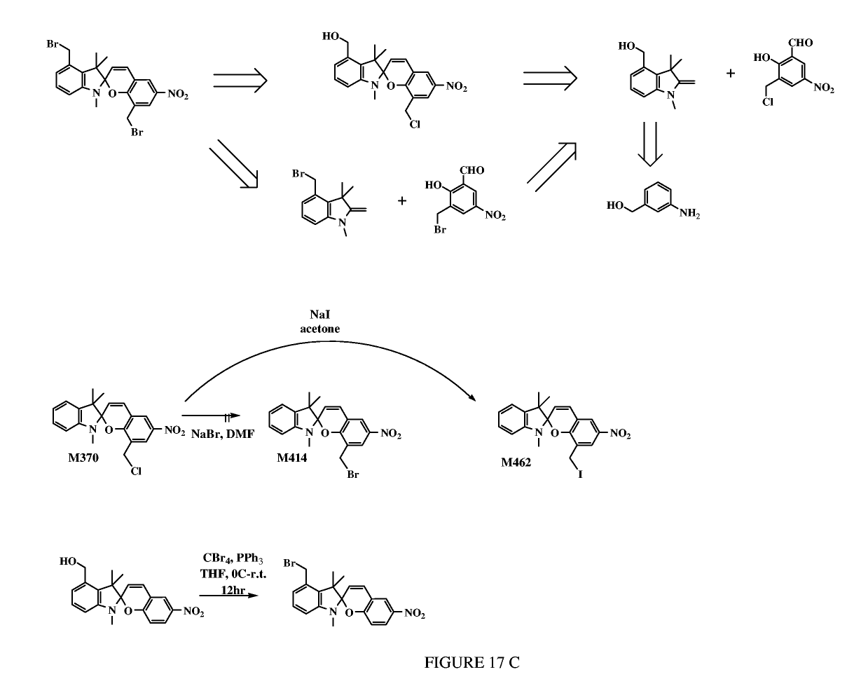

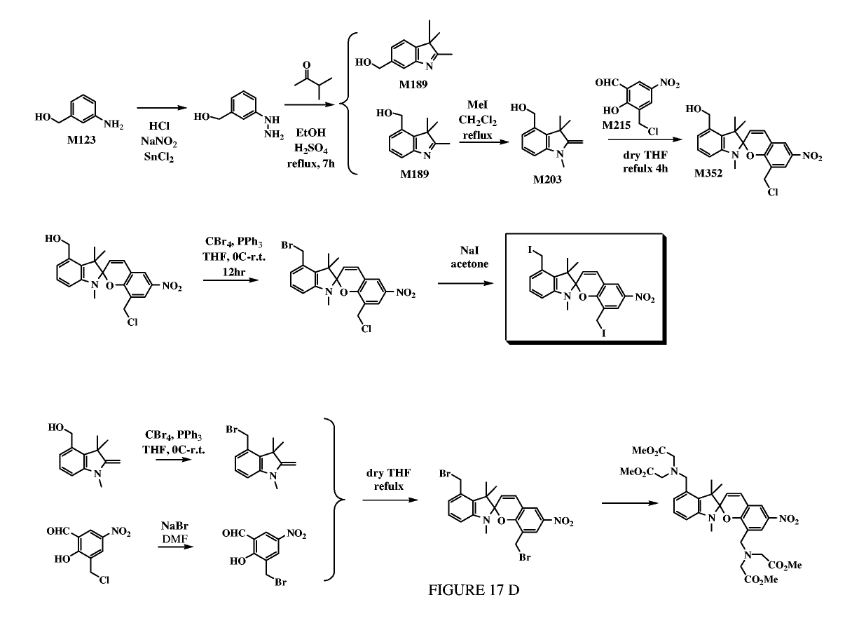

Combinatorial libraries are illustrated in FIGS. 14 and B and FIGS. 17 A, B, C and D. Thiol reactive probes and any optical switch compound such as nitrospirobenzopyran and spironaphthoxazines may be further substituted to generate derivatives, thereby greatly increasing the combinatorial library of these compounds. Thus, for example various substitutions on the spironaphthoxazine ring may greatly expand the number of compounds in the library and may be useful in increasing the absorption wavelength of the SP and MC states—and in increasing functionality of these compounds, such as by incorporating thiol reactive groups in a calcium chelator. Such permutation and combinations will eventually lead to and allow for multiplexed optical control—which can be seen most clearly in FIG. 17. As shown in this figure, the MC absorption spectra for the nitrospirobenzopyran (NSB) and the spironaphthoxazine (SNZ) are well separated (by 100 nm) such that they can be excited independently. Independently excited, NSB and SNZ in turn may be exploited in applications within cell biology, proteomics, genomics and in nanotechnology to control multiple molecular species e.g. calcium ions and some other protein activity, surface based control of two or more metal ions bound to two classes of optical switch chelate based on NSB and SNZ.

Overall, the present invention envisions a new class of chemical library for drug screening. The ability to synthesize>million member library of SBP and SNZ compounds using the approach outlined in this patent represents new opportunities for drug screening and discovery. Specifically the drugs allow for:
(1) Complex structures in the SP states with orthogonal disposed aromatic rings harboring diverse functional groups
(2) Facile synthesis for members of the library from separate libraries of indolines and salicylaldehydes—incorporation of reactive groups e.g. the thiol reactive probes described herein, can be used to expand the library—i.e. react with a library of small molecule mercaptans. For example, the inventors have 16 indoline and 6 salicyladehyde building blocks in the exemplary library that can make 96 different SBP and 96 SNZ compounds. Moreover, since there are at least 1000 different small molecule mercaptans commercially available—this will lead to a 2 million member library—incorporation of new indolines in this combinatorial approach. Other common techniques may be used to expand the library. Thus for example, those compounds incorporating an extra phenyl ring may be used to expand the library. Library of calcium chelates is depicted in FIG. 14, and synthesis schemes of these chelates and their intermediates for use in optical probes is depicted in FIGS. 17 A, B, C and D.
(3) In the present invention, false positives can be eliminated rapidly by switching the SP to MC state—given the dramatic difference in the structures between the SP and MC states it is unlikely that a true target will bind to both SP and MC states, whereas a false positive will appear as positive for both the SP and MC states.

### EXAMPLE IV

### Design, Synthesis and Characterization a New Class of Ca²⁺ Optical Switch

Cell protrusion is characterized by fluctuations in the concentrations and activities of, and/or interactions between, membrane receptors, Rac1, Ca²⁺, PIP₂, cofilin and actin filament barbed end capping proteins. These dynamic events are confined to the lamellipodium and are somehow coupled to the rapid and polarized polymerization of actin filaments in the vicinity of the activated membrane receptor (Barkalow et al, 1996;Hartwig et al, 1995; Machesky & Insall, 1999; Cox et al, 1997; Pollard, 2003; Ghosh et al, 2004; Vallotton et al, 2004). A major challenge in cell motility research is to develop innovative approaches to study the temporal and spatial regulation of these molecular events within the ˜6 fL volume of a typical lamellipodium (3 μm×10 μm×0.2 μm; Abraham et al, 1999; Zhang et al, 2002)—these techniques must be capable of detecting, mapping and resolving interactions for fewer than 1000 barbed ends within the few seconds it takes to form a protrusion.

The late Fred Fay and his colleagues and others have shown that the increase in cell Ca²⁺ shortly after the activation of membrane receptors correlates with a dramatic increase in the rate of actin filament polymerization and contraction of actomyosin at the cortex (Brundage et al, 1991; 1993; Walker et al, 2001; Hendey et al, 1993; Maxfield, 1993). While Ca²⁺ activates many cytoskeleton proteins, most notably calmodulin (Hahn et al, 1992), the Ca²⁺-dependent barbed-end capping proteins, such as CapG and Gelsolin are likely to be the primary targets for cell protrusion (Young et al, 1994)—deletion of either or both of genes leads to a dysfunctional regulation of protrusion in macrophage cells (Witke et al, 1995; 2001). PIP₂ is also associated with the regulation of actin filament polymerization (Janmey, 1994; Yin & Janmey, 2003) and is believed to dissociate capping proteins from their complexes with the barbed-end (Schafer et al, 1996; Cooper & Shafer, 2000) and trigger the actin filament-mediated comet-like motion of vesicles in cells (Rozelle et al, 2000). The focus of this proposal is to understand how changes in the concentration, interactions and activities of Ca²⁺ and PIP₂ are integrated in the lamellipodium and coupled to the regulation of the barbed end of actin filament, actin polymerization and cell protrusion.

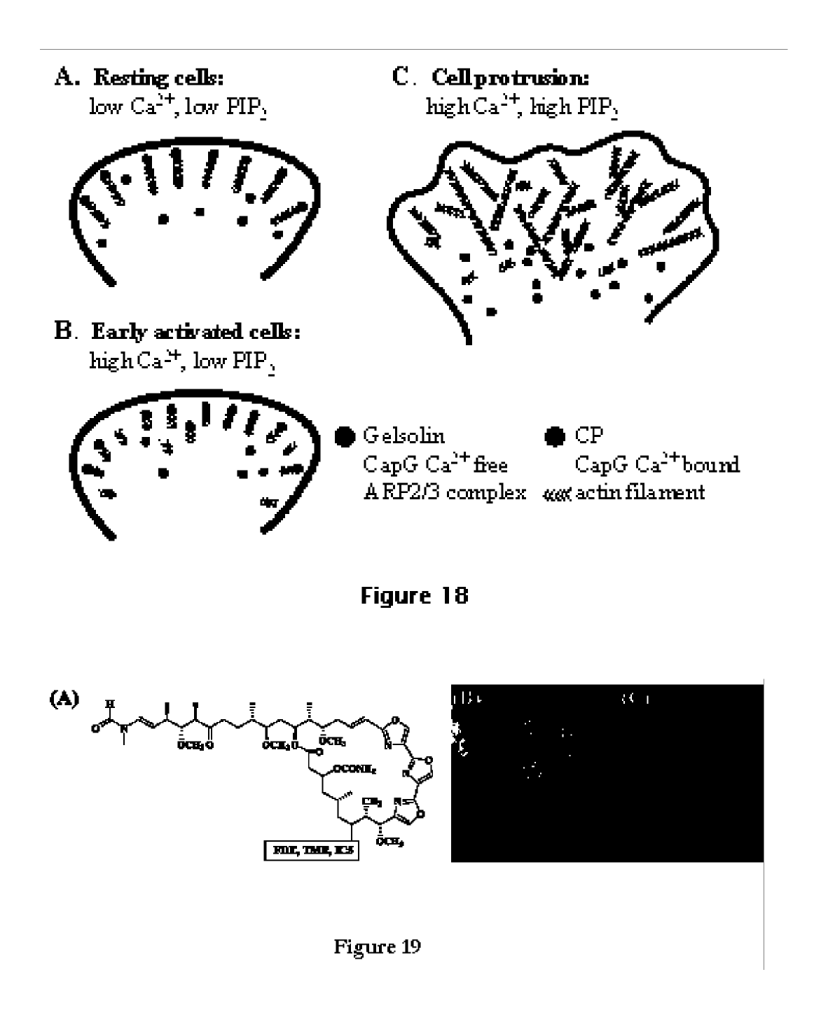

A framework to understand the roles of Ca²⁺, PIP₂ and key cytoskeleton-associated proteins in the regulation of cell protrusion is shown in FIG. 18.

Regulation of molecular interactions at the barbed-end of the actin filament: On the basis of the model shown in FIG. 1A, we suggest that the fast growing, barbed-end of the actin filament in resting cells is prevented from polymerizing by a specific interaction with CapG, capping protein (CP) or Gelsolin (DiNubile & Southwick, 1986; Yin & Janmey, 2003; Hug et al, 1995; Mejillano et al, 2004). Dissociation of these proteins is believed to precede the explosive polymerization of actin filaments that accompanies the formation of lamellipodia and ruffles (FIG. 1C).

CapG:CapG is a 40 kD, Ca²⁺ regulated barbed-end capping protein that is found at high levels in macrophage cells where it regulates actin mediated membrane ruffling during phagocytosis and motility. CapG, unlike Gelsolin, does not sever actin filaments. In vitro studies show that CapG binds to the barbed-end of the actin filament in a Ca²⁺-dependent fashion with a k_{d} of 1 μM (Southwick & DiNubile, 1986). Unlike Gelsolin and CP, the complex between the barbed end and CapG dissociates at low Ca²⁺. The interaction of CapG with actin is perhaps the simplest and the most tractable experimental model system to study the regulation of actin filament barbed-ends during protrusion since: (1), CapG is a monomeric protein that exhibits a straightforward interaction with G- and F-actin (Yu et al, 1990); (2), CapG binds to actin only in the presence of Ca²⁺ and does not sever filaments; (3), CapG binds to the same site on G- and F-actin, which facilitates structural and mechanistic investigations on the regulation of this complex (Tanaka et al, 2003); (4), the CapG-actin complex, like other capping proteins, is regulated by PIP₂; (5), The regulation of the CapG-actin complex can be studied in macrophage cells derived from CapG and/or Gelsolin-null mice, which are known to exhibit defects in ruffling and protrusion (Witke et al, 2001).

PIP₂: PIP₂ regulates actin filament dynamics by dissociating Gelsolin, CapG, and CP from the barbed end of the actin filament (Yin, 1987; Yin & Janmey, 2003; Hartwig et al, 1995; 1996; Shafer & Cooper, 2000; Sun et al, 1999). Consistent with this mode of regulation, platelet cell activation is accompanied by a rise in PIP₂, which precedes actin polymerization and protrusion (Bartalow et al, 1995). Further support for this model comes Botelho et al (2000), who showed that PIP₂ regulates actin polymerization during phagocytosis, and a study involving the PI's laboratory (Rozelle et al, 2000), that equated an increase in cellular PIP₂ with actin polymerization and the motility of vesicles in cells.

Ca²⁺-independentregulation of barbed ends: Ghosh et al (2004) has shown that free barbed-ends are generated in the lamellipodium by the weak severing activity of cofilin. This discovery is important because it provides an explanation for the large increase in the number of free barbed ends during cell protrusion that cannot be accounted for by the one time severing/capping activity of gelsolin (Bartalow et al, 1995; Yin &Janmey, 2003). Cofilin, on the other hand, may engage in multiple, Ca²⁺-independent actin filament severing events that generate free barbed ends that are capped by CP (Mejillano et al, 2004), or else lead to a Ca²⁺-independent polymerization of actin filaments. We will investigate the role of Ca²⁺-independent generation of free barbed ends in cell protrusion using light direct activation of a constitutively active caged cofilin in cells and using new fluorescent KabC probes (Tanaka et al, 2003) to image the distribution of free barbed ends by uncaging cofilin in cells.

Functional redundancy:The mechanisms underlying Ca²⁺ and PIP₂-mediated regulation of molecular interactions at the barbed-end during cell protrusion are not fully understood—these investigations are further complicated because CapG, CP, radaxin and gelsolin cap filament barbed ends using the same site on actin (Schafer et al, 1996; Tsukita et al, 1989; Klenchin et al, 2003; Tanaka et al, 2003; Kim et al, 2004). On the other hand, gene knock out studies show that CapG, gelsolin and CP each play essential roles in regulating cell protrusion and motility (Mejillano et al, 2004; Witke et al, 1995; 2001). We will address the potential problem of functional redundancy by focusing our studies on the Ca²⁺-dependent capping interactions of CapG in macrophage cells derived from CapG-null, Gelsolin-null, and Gelsolin/CapG-double-null mice (Dr. Walter Witke has agreed to provide the PI with these mice and protocols to isolate and culture primary macrophages. We will use the RNAi approach of Mejillano et al (2004) to suppress CP function in cells. In addition to macrophage cells, we will conduct key experiments within the highly protrusive Neuro-2a neuroblastoma (Rosner et al, 1995). These cells have large and dynamic lamellipodia and are amenable to transfection and microinjection.

A biophotonics approach to understand the molecularregulation of cell protrusion: We will employ a biophotonics approach to correlate changes in the interactions between Ca²⁺, PIP₂ and CapG with events at the barbed end of the actin filament. These studies, which involve collaborations with Prof. Peter So and Yuling Yan, will involve optimizing imaging techniques including Foerster resonance energy transfer (FRET), fluorescence recovery after photobleaching (FRAP), photoactivation of fluorescence (PAF) and speckle (Yan & Marriott, 2003; Lanni & Ware, 1984; Theriot & Mitchison, 1991; Waterman-Storer & Salmon, 1997) for time-correlated optical perturbations of caged compounds and proteins (Adams et al, 1997; Marriott et al, 2003) and our new optical switches.

Rapid and reversible, optical switching of Ca²⁺: Ca²⁺-transients can be artificially generated within cells using light-directed activation of caged Ca²⁺ chelators (Adams et al, 1997; Ellis-Davies, 2003). However, the relatively slow and irreversible photochemistry of the 2-nitrophenyl caging groups and their toxic photoproducts limit the usefulness of this approach. The ideal Ca²⁺ perturbation probe would be one whose affinity for Ca²⁺ changes rapidly and reversibly via optical transitions between two switch states. Transitions between these two states and the accompanying change in their Ca²⁺ binding affinity would more closely mimic the nature of Ca²⁺ transients that exist in cells. We are designing optical switches based on the spirobenzopyran photochrome that incorporate this desired property (FIG. 18)—spirobenzopyrans undergo rapid and reversible transitions between a colorless spiro (SP) state and a colorful merocyanine (MC) state (Inouye, 1994; Giodarno et al, 2002; Medintz et al, 2004). These optical switches are controlled by exciting the SP state with 365 nm light (SP to MC) and the MC state with 546 nm light (MC to SP). The SP and MC states of the proposed Ca²⁺ optical switches (Scheme 1; FIG. 8) should exhibit a considerable difference in their Ca²⁺-binding affinities (FIG. 8). The advantage of our optical switching approach for perturbing cell Ca²⁺ compared to caged Ca²⁺ chelators include: 1), Optical transitions between the SP and MC states of the switch are fully reversible; 2), Optical transitions between the two states of the switch are rapid (11 μs) and proceed with high quantum efficiencies; 3), Optical transitions between the switch states do not release photoproducts; 4), The absorption band for the MC to SP transition can be tuned over a broad wavelength and are red shifted so as to limit interference from other optical probes in the sample—control of the MC to SP action spectrum provides an opportunity to selectively control multiple switches within a single cell; 5), a single optical switch is used to sequester and release Ca²⁺; 6), The probes incorporate a fluorescence readout (MC state) that serves to check the status and operation of the switch while providing a means to determine the rate for transitions between the two switch states.

Rapid and reversible modulation of protein interactions using optical switches: Spirobenzopyran conjugates provide an opportunity to optically control the interactions and activities of specific proteins in complex environments (Willner et al, 1994)—our interest in these probes center on developing optical switches to control interactions between Ca²⁺ and barbed end capping proteins during cell protrusion. We have found that the action spectrum of these switches can be tuned over a wide wavelength range using spirobenzopyran and spironaphthoxazine based reagents. Since the MC states of spirobenzopyran and spironaphthoxazine protein conjugates can be selectively controlled using different excitation wavelengths, this feature will allow control of multiple optical switch conjugates and their interactions and activities within a cell. We propose to develop a simple approach to prepare optical switch conjugates focusing our initial studies on the simple Ca²⁺ binding protein parvalbumin.

Specifically we propose to position the MC state of a thiol reactive spirobenzopyrans to a specific site on a engineered parvalbumin where it engages in MC—, but not SP, dependent interactions that block Ca²⁺ binding to the optical switch conjugate (schematized in FIG. 1).

New probes to image molecular events at the barbed-end of actin filaments during cell protrusion: The studies outlined in this proposal require the development of fluorescent probes that selectively report on changes in the distribution of free barbed ends during cell protrusion. Ideally, these probes would act as indicators of uncapping events at the barbed end. We have previously shown that actin-targeted, macrolide drugs typified by KabC (FIG. 6A) function as unregulated biomimetics of Gelsolin, while the stable G-actin-KabC complex functions as an unregulated barbed-end capping protein without severing activity (Tanaka et al, 2003; Klenchin et al, 2003). At low concentrations of fluorescent KabC used in cell studies, the probe will bind directly to G-actin and should function as a high-contrast probe for quantifying the distribution and number of free barbed ends within the cell. We aim to show how the TMR and FDE KabC probes provide unrivalled contrast in probing molecular interactions at the barbed-end of the actin filament—these probes will be used to understand the roles of Ca²⁺ and PIP₂ in the spatial and temporal regulation of the CapG-barbed-end complex during cell protrusion.

Design, synthesize and characterize optical switches for Ca²⁺

The ideal probe to modulate Ca²⁺ would incorporate both Ca²⁺ sequestering and release activities that are controlled through rapid and reversible, light-driven reactions without the release of secondary products. As part of a new program to develop such probes, we introduce a family of tetracarboxylic chelating reagents based on the spirobenzopyran photochrome that undergo rapid and reversible, light-directed transitions between two stable isomeric states, a colorless spiro-(SP) state and a colorful merocyanine (MC) state (Inouye, 1994)—these probes are designed to exhibit different affinities for Ca²⁺ in the SP and MC states. We suppose that this property can be introduced by positioning two pairs of N-linked carboxyl groups on the optical switch with a geometry that favors Ca²⁺ binding in either the SP or the MC state. In the case of the compound X, the ester precursor of the tetracarboxylic chelator, we reasoned that the close proximity of the two pairs of carboxyl groups in the planar MC state would favor Ca²⁺ binding over the SP state—we reasoned that the opposite would be true for compound VIII as indicated in FIG. 10. The synthetic approaches used in these studies are summarized in Scheme IV. All compounds are generated in excellent yield and are fully characterized by NMR and mass spectrophotometry.

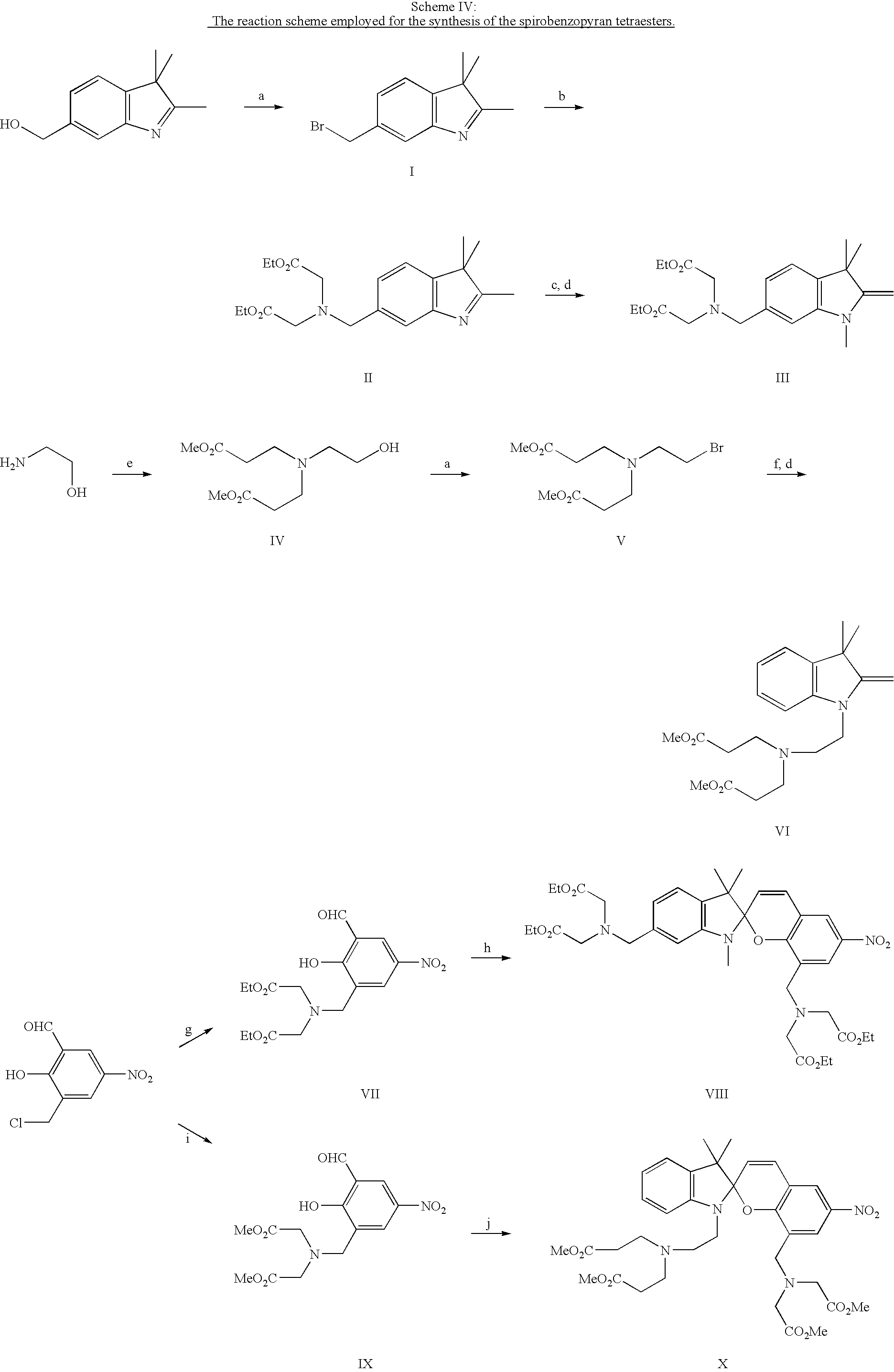

Reagents: (a) PPh₃, CBr₄, CH₂Cl₂; (b) diethyl iminodiacetate, K₂CO₃, THF, reflux; (c) methyl iodide, CHCl₃, reflux; (d) 0.5 N NaOH; (e) methyl acrylate, MeOH, 40° C.; (f) 2,3,3-trimethyl-3H-indole, CH₃CN, reflux; (g) diethyl iminodiacetate, Et₃N, THF, reflux; (h) III, EtOH; (i) dimethyl iminodiacetate hydrochloride, Et₃N, THF, reflux; (j) VI, EtOH.

Analytical Data:

6-(bromomethyl)-2,3,3-trimethyl-3H-indole (I): Yield: 17%; MS(EI): 253(7), 251(M⁺,7), 172(M⁺—Br,62), 83(100); HRMS(EI): M⁺251.0320 (calc. 251.0310); ¹H NMR (CDCl₃) δ1.31 (s, 6H), 2.30(s, 3H), 4.57(s, 2H), 7.25-7.28(m, 2H), 7.56(s, 1H).

6-[N′,N′-bis(ethyloxycarbonylmethyl)aminomethyl]-2,3,3-trimethyl-3H-indole (II): Yield: 54%; MS(EI): 360(M⁺,5), 287(56), 273(15), 172(100); HRMS(EI): M⁺360.2046 (calc.360.2049); ¹H NMR(CDCl₃) δ1.28(t, J=7.2 Hz, 6H), 1.31(s, 6H), 2.30(s, 3H), 3.58(s, 4H), 3.97(s, 2H), 4.18(q, J=7.2 Hz, 4H), 7.24(d, J=7.5 Hz, 1H), 7.30(d, J=7.5 Hz, 1H), 7.51(s, 1H).

6-[N′,N′-bis(ethyloxycarbonylmethyl)aminomethyl]-1,3,3-trimethyl-exo-methyleneindoline (III):Unstable, used for the next reaction without purification and characterization.

2-[N,N-bis(methyloxycarbonylethyl)amino]ethanol (IV): Used for the next reaction without purification. ¹H NMR (CDCl₃) δ2.48(t, J=6.5 Hz, 4H), 2.60(t, J=6.5 Hz, 4H), 2.80(t, J=7.7 Hz, 2H), 3.60(t, J=7.7 Hz, 2H), 3.69(s, 6H).

N,N-bis(methyloxycarbonylethyl)-2-bromoethylamine (V): Yield: 91%; MS(EI): 297(7), 295(M⁺,7), 224(88), 222(89), 202(100); HRMS(EI): M⁺295.0408 (calc. 295.0419); ¹H NMR(CDCl₃) δ2.47(t, J=6.5 Hz, 4H), 2.84(t, J=6.5 Hz, 4H), 2.85(t, J=7.7 Hz, 2H), 3.35(t, J=7.7 Hz, 2H), 3.69(s, 6H).

N-[N′,N′-bis(methyloxycarbonylethyl)aminoethyl]-3,3-dimethyl-exo-methyleneindoline (VI): Unstable, used for the next reaction without purification and characterization.

3-[N,N-bis(ethoxycarbonylmethyl)aminomethyl]-5-nitrosalicylaldehyde (VII): Used for the next reaction without purification. ¹H NMR (CDCl₃) δ1.28(t, J=7.0 Hz, 6H), 3.57(s, 4H), 4.05(s, 2H), 4.20(q, J=7.0 Hz, 4H), 8.27(d, J=2.9 Hz, 1H), 8.55(d, J=2.9 Hz, 1H), 10.28 (s, 1H).

Spirobenzopyran 8,6′-tetraester (VIII): Yield: 22% based on II; HRMS(ESI): [M+Na]⁺747.3202 (calc.747.3217); ¹H NMR (CDCl₃) δ1.18 (s, 3H), 1.19 (t, J=7.1 Hz, 6H), 1.26 (s, 3H), 1.28 (t, J=7.1 Hz, 6H), 2.70 (s, 3H), 3.26 (d, J=7.6 Hz, 2H), 3.33 (d, J=7.6 Hz, 2H), 3.57 (s, 4H), 3.63 (s, 2H), 3.89 (s, 2H), 4.06 (q, J=7.1 Hz, 4H), 4.18 (q, J=7.1 Hz, 4H), 5.85 (d, J=10.3 Hz, 1H), 6.63 (s, 1H), 6.81 (d, J=7.5 Hz, 1H), 6.91 (d, J=10.3 Hz, 1H), 6.98 (d, J=7.5 Hz, 1H), 7.93 (d, J=2.5 Hz, 1H), 8.18 (d, J=2.5, 1H).

3-[N,N-bis(methoxycarbonylmethyl)aminomethyl]-5-nitrosalicylaldehyde (IX): Used for the next reaction without purification. ¹H NMR (CDCl₃) δ3.62(s, 4H), 3.78(s, 6H), 4.12(s,2H), 8.35(d, J=2.7 Hz, 1H), 8.61(d, J=2.7 Hz, 1H), 10.36 (s, 1H).

Spirobenzopyran 8,1′-tetraester (X): Yield: 10% based on V; HRMS(ESI): [M+H]⁺697.3057 (calc.697.3085);

¹H NMR (CDCl₃) δ1.71(s, 3H), 1.26(s, 3H), 2.37(t, J=7.2 Hz, 4H), 2.50-2.65(m, 2H), 2.74(t, J=7.2 Hz, 4H), 3.14-3.25(m, 2H), 3.31(s, 4H), 3.61(s, 6H), 3.62(s, 6H), 3.61-3.65(m, 2H), 5.88(d, J=10.6 Hz, 1H), 6.57(d, J=7.6 Hz, 1H), 6.85(dd, J=7.6, 7.6 Hz, 1H), 6.93(d, J=10.6 Hz, 1H), 7.07(d, J=7.6 Hz, 1H), 7.16(dd, J=7.6, 7.6 Hz, 1H), 7.94(d, J=2.7Hz, 1H), 8.16(d, J=2.7Hz, 1H).

De-esterification: At the time of submission we have not established conditions for the quantitative removal of all four methyl or ethyl ester groups in compounds VIII and X. This will be necessary to determine the Ca²⁺-bimding constants for the SP and MC states. We have established however, that the spirobenzopyran group is sensitive to the basic condition that is usually employed for ester hydrolysis. We are now employing acid hydrolysis as an alternative since the SP and MC states are stable in acid medium (Raymo et al, 2004). Even more recently, we have achieved the synthesis of the t-butyl ester compound closely related to compound X, which should yield the de-esterified form in the presence of trifluoroacetic acid.

Cell loading: The carboxylic esters of the putative Ca²⁺ chelators facilitate the entry of the optical switch into cells. In the case of the ethyl ester (VIII), the fact that the probe remains within the cell after washing with medium suggests that one or more of the ester groups are removed by intracellular esterases. Loaded cells do not exhibit a red fluorescence until they are irradiated with a short pulse of 365 nm light (FIG. 7B), which we argue arises from the MC state. MC fluorescence is extinguished following irradiation with 546 nm light sue to the excited state photoisomerization to the colorless SP state. Alternate irradiation of loaded cells with 365nm and 546 nm light leads control of MC fluorescence (FIG. 7B) over many irradiation cycles. If the SP and MC states do show differences in their kd for Ca²⁺, then the high fidelity optical switching between the SP and MC states seen for fluorescence (FIG. 7B) should allow the investigator to control Ca²⁺ binding in the switch using light.

Local Increases in Ca²⁺ are Coupled to the Activation of Actin Filament Polymerization and the Global Response of Cell Protrusion

Design, Synthesis and Application of Fluorescent Barbed-End Probes:

7-(4-aminomethyl)-1H-1,2,3-triazol-1-yl analogue of kabiramide C (AMT-KabC) KabC was converted into 7-azido KabC via Mitsunobu reaction (Ko, 2002) by using hydrazoic acid as nucleophile in the presence of PPh₃ and DIAD under nitrogen atmosphere. Then 7-azido KabC was reacted with 3-(fluoren-9-yl-methoxycarbonyl)aminopropyne in the presence of catalytic amount of copper (I) iodide and Et₃N (Horne et al, 2003, Tornoe et al, 2002, and Rostovsev et al, 2002) to afford amino protected KabC. This compound was confirmed by the presence of aromatic proton signals of Fmoc in the ¹H NMR spectrum. Deprotection of Fmoc with 20% piperidine in dry CH₂Cl₂ gave AMT-KabC (FIG. 6). The ¹H NMR spectrum confirmed the triazole proton (●) signal at δ 7.47 ppm and methylene proton (*) signal at δ 4.02 ppm. Compounds were confirmed by ¹H NMR and HRMS (ESI). Following Scheme V depicts synthesis of an amino containing kabiramide (5) for coupling to fluorescent probes.

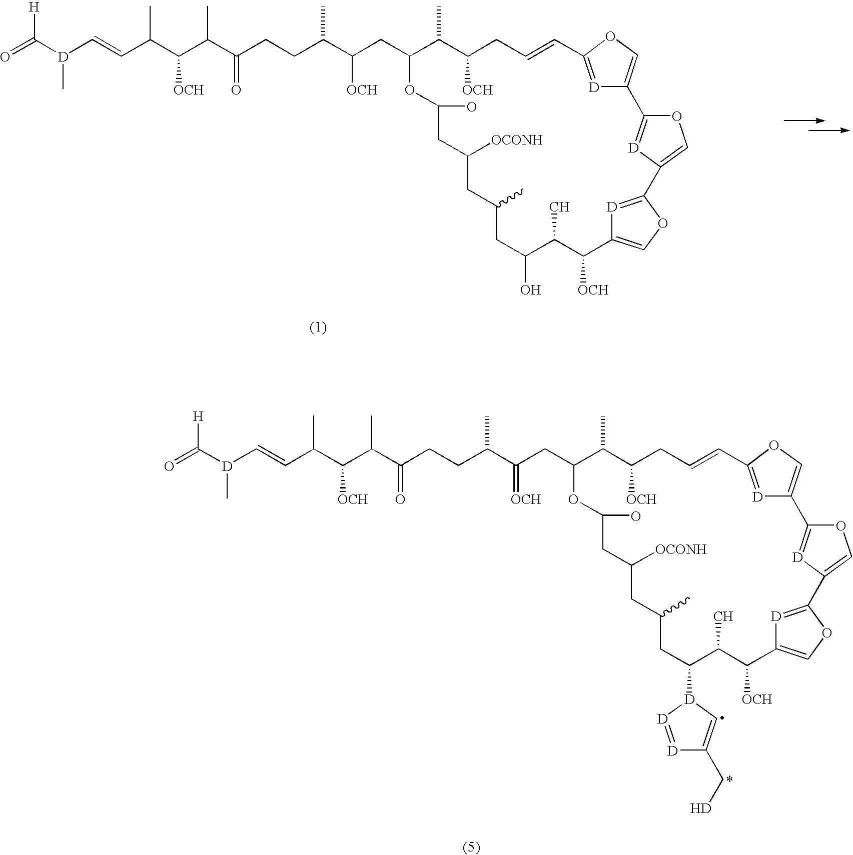

Fluorescent KabC: New probes for the barbed end of actin filaments The KabC derivatives of tetramethylrhodamine (TMR), fluorescein diester (FDE) and IC5 (FIG. 7A) were prepared from the corresponding succinimide esters (Molecular Probes; Dojindo) as described for TMR-KabC: a solution of 5-(and-6)-carboxytetrametylrhodamine, succinimidyl ester (0.6 mg, 1.14 μmole) in DMSO 100 μL was added to a solution of AMT-KabC (1 mg, 0.98 μmole) in dry CH₂Cl₂ 100 μL. The reaction mixture was stirred at room temperature under nitrogen atmosphere. After 8 hours, the mixture was concentrated and isolated by preparative TLC on silica gel (CH₂Cl₂:MeOH=20:3) and ODS (MeOH:H₂O=7:3) to give the tetramethylrhodamine derivative of KabC

FDE-KabC readily permeates the plasma membrane of living cells and fluoresces only after one or both acetate groups are hydrolyzed by intracellular esterases—a robust fluorescein fluorescence is visible within a few minutes of cell loading. The TMR—KabC traverses the plasma membrane and is retained floowoing washing with fresh medium. Confocal imaging of the FDE-KabC (FIG. 19B) and TMR-KabC (FIG. 19C) probes within Hela cells shows that the emission of both probes is focused at the plasma membrane and especially at sites of dynamic cell protrusion (FIGS. 19B, C). Since stress fibers and other stable actin filament containing structures are not labeled to any significant degree by these probes and from the findings of our previous studies (Klenchin et al, 2003 and Tanaka et al, 2003), we suggest that the KabC probe binds to the free barbed ends that are generated at sites undergoing active actin polymerization.

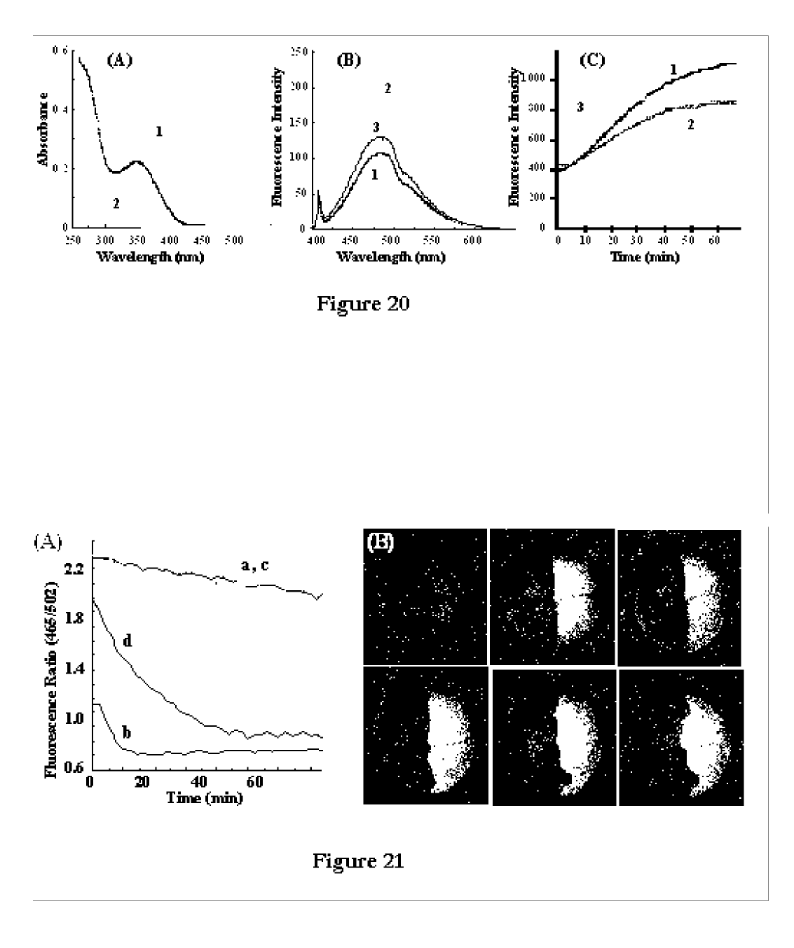

Preparation and characterization of caged CapG: Caged CapG was prepared using a modification of our published method for caging actin and profilin (Marriott, 1994; Marriott et al, 2003). In brief a purified solution of CapG (1 mg/ml) was dialyzed against 20 mM borate buffer, pH 8.5 overnight. The protein was centrifuged and the OD_{280 nm} measured to determine the protein concentration (FIG. 8A). A 0.1 M acetone solution of NVOC—Cl was freshly prepared and added to the CapG with vigorous vortexing to a final concentration of 1 mM. This was usually realized by 2-3 additions over a 5-minute period. After 30 minutes at room temperature in the dark, the protein was centrifuged and dialyzed against 2×500 ml of G-buffer overnight. The absorption spectrum of the protein was recorded using the dialysis buffer as a reference and the amount of NVOC was determined by measuring the A_{350 nm} (E_{350 nm}=5,000 M⁻¹cm⁻¹). The concentration of protein was determined using the Bio-Rad Bradford assay. The introduction of 3-4 NVOC groups was sufficient to inhibit the formation of G-actin-CapG using two independent assays based on sensitivity of Prodan-actin to CapG binding. In FIG. 20B, CapG binding to Prodan-G-actin is seen to shift the emission spectrum, from 496 nm to 465 nm, and is accompanied by a 1.5 fold (FIG. 20B, curve 1)—this signal can be used to analyze the effects of CapG on actin polymerization kinetics as shown in FIG. 20C. Thus while CapG inhibits forms a stable complex with G-actin (FIG. 20B, curve 2; k_{d} of 100 nM data not shown) and inhibits the polymerization of G-actin (FIG. 20C) the caged CapG described above binds poorly to G-actin and has a limited effect on the rate of actin polymerization (FIGS. 20B, curve 3).

Light Directed Activation of Constitutively Active Caged Cofilin

Caged cofilin (constitutively active S3A) was prepared independently of Ghosh et al (2004) using our standard NVOC—Cl approach (Marriott, 1994) that is described above for caged CapG. Constitutively active cofilin increases the rate of F-actin depolymerization as seen in the fluorescence emission ratio (465/502 nm) of Prodan actin following dilution below the critical concentration (FIG. 21, curve b). The same kinetic assay was used to show that caged cofilin (curve c) did not accelerate the rate of F-actin depolymerization compared to the control F-actin (curve a). Uncaged cofilin restores a substantial fraction of the cofilin activity as seen in curve d. We found that 3-5 NVOC groups were sufficient to inhibit cofilin activity. In vivo analysis of caged cofilin is seen in a study on development in fertilized xenopus oocytes shown in FIG. 21B. Oocytes were microinjected with caged fluorescein dextran (Molecular Probes) and concentrated caged cofilin (5 mg/ml) at the 4 cell stage—we estimate that the intracellular concentration of cofilin is the two injected cells is between 30˜50 μM. Irradiation of all four cells in the embryo with 365 nm light for 2 seconds generated fluorescein and green fluorescence, and by implication cofilin, from their caged precursors in the two injected cells. This level of irradiation has no effect on the development and the very small amount of photoproduct released does not perturb cell function (Roy et al, 2001). While the non-injected cells developed normally, as indicated by normal cell divisions, the two cells injected with fluorescein and cofilin failed to undergo further divisions (FIG. 21b). We conclude from these studies that the increased concentration of cofilin generated by uncaging inhibits actin filament dynamics and cytokinesis (Bamburg, 1999). Control experiments using constitutively active cofilin produce inhibited cytokinesis whereas wild type cofilin, which is inactivated by phosphorylation at Ser-3, has no effect on cell division. Microinjection of caged fluorescein and subsequent uncaging using 365 nm light has no effect on cell division. These results are consistent with studies on the cofilin-mediated inhibition of cytokinesis (Bamburg, 1999) and changes of actin filament dynamics in motile cells (Ghosh et al (2004).

Methodology:

Relationships between Ca²⁺-mediated capping activities, actin filament dynamics and cell protrusion are studied by locally triggering: (a), cell Ca²⁺ using caged and optical switch Ca²⁺ probes (Adams et al, 1997; Ellis-Davies, 2003); (b), caged and optical switch conjugates of CapG; (c), Caged Rac1; (d), caged cofilin; (e), caged PIP₂. Fluorescent tags attached to KabC, CapG and actin in CapG/Geloslin double null macrophage cells, NIH 3T3 and the highly protrusive, Neuro-2a cell line, will be used to map events at the barbed-end of the filament and the rate of actin polymerization in response to a rise in cell Ca²⁺ and PIP₂. These studies also integrate phase contrast images that collectively allows correlation of local changes in cell-Ca²⁺ are coupled to the regulation of actin filament polymerization and integrated to achieve the global response of cell protrusion.

Methodologies for Designing, Synthesis and Characterization of Optical Switches for Reversibly Modulating Ca²⁺

Preliminary results described the synthesis of potential Ca²⁺-optical switches—these probes were designed to place two different N-linked pairs of carboxylic esters at different sites the same spirobenzopyran scaffold. We envision using these optical switches to rapidly and reversibly modulate Ca²⁺ in cells according to FIG. 8. This requires that we prepare the free carboxylic acids (or K⁺) forms of the probes and determine their affinities for Ca²⁺ and Mg²⁺ in the SP and MC states.

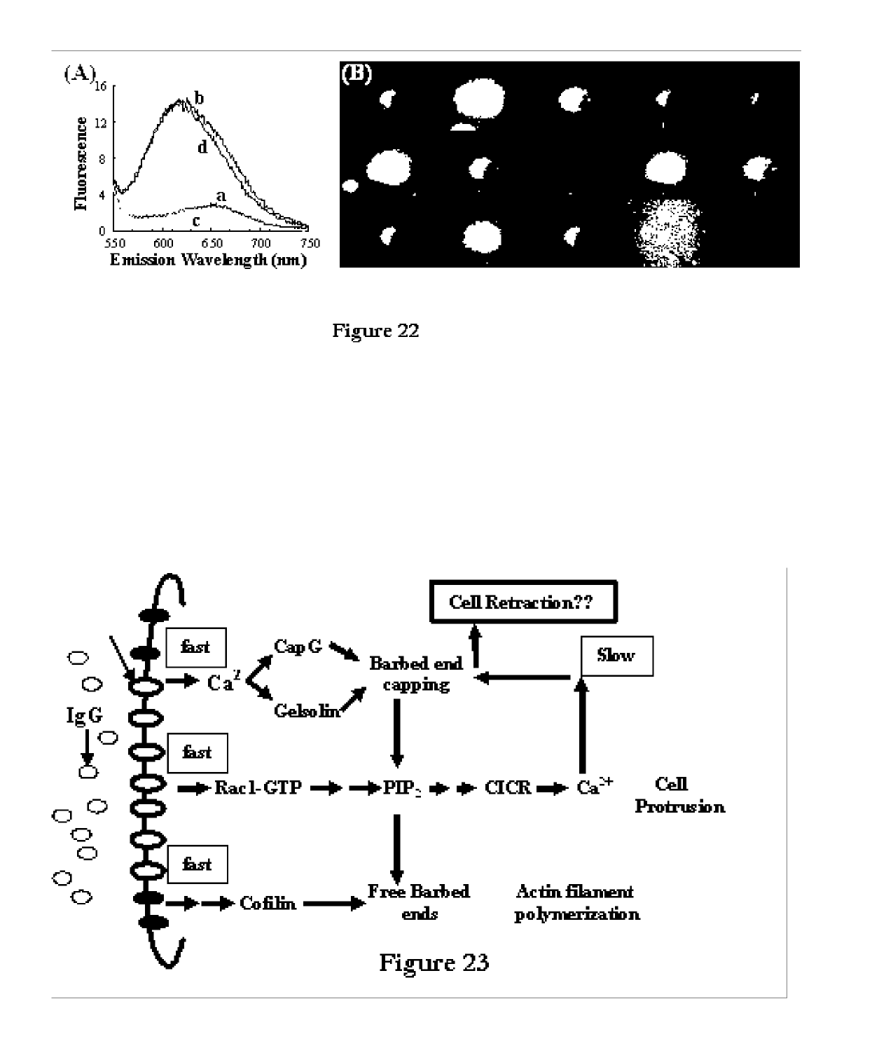

These measurements are made using a competition binding assay with fluorescent divalent metal ion indicators (Fluo-3, X-Rhod-1 and X-Rhod-2; Molecular Probes). Recently established nitrospirobenzopyran probe has been listed as sensitive to the basic conditions required to remove saponification of the esters. Other reagents may also be used to hydrolyze these esters. These studies may be limited to using acid hydrolysis (the probe is stable in acid medium; Raymo et al, 2004) and, since we know at least some esters are cleaved in vivo by intracellular esterases (FIG. 22), we will also employ commercially available esterases covalently linked to beads (Sigma). Finally we have recently synthesized the t-butyl ester of a closely related analog of compound X and we have preliminary data to suggest that the t-butyl group is efficiently cleaved in acid media. In addition we will use light directed cleavage of 2-nitrophenyl esters of Vil and X (Ottl et al, 1998).

A family of optically switches for Ca²⁺ that exhibit the following properties may be generated, wherein:
(1) Ca²⁺-binding constant between the SP and MC states of >10-fold; Ca²⁺ versus Mg²⁺>100-fold
(2) Ca²⁺-binding constants within the family of probes that vary from 100 nM to 100 μM
(3) MC-state absorption maxima between 500˜650 nm
(4) Rate constants for Ca²⁺-binding and release as high as 10⁶ s⁻¹

Optimization of the Ca²⁺-binding constants for the SP and MC states, discrimination between Ca²⁺ and Mg²⁺ and rate constants for optical switching can be realized by varying the coordination geometry of the two pairs of carboxylic acids. For compound Vil these groups are close in the SP state and far apart in the MC state and we envision that the SP state would bind more tightly to Ca²⁺ than the MC state. On the other hand the very short distance between the two pairs of carboxylic acids in the SP of compound X, and the more optimal distance observed in the MC state, coupled with the employment of longer and more flexible linker groups is designed to improve the Ca²⁺ affinity of MC over SP. The MC state of compound X positions the pairs of carboxyl groups at the same molecular distance on the planar aromatic ring as those in Fluo-3.

The combinatorial approach to the synthesis of these optical switches allows is to mix and match different functional groups within libraries of indolines and salicylaldehydes derivatives. Thus by selecting appropriately di-carboxyl indoline and salicylaldehyde reagents, we can rapidly and systematically control the location and geometry of carboxyl groups on the spirobenzopyran as well as the length and flexibility of the linker between the nitrogen atom and the pair of carboxyl groups (indicated as* in FIG. 10), and/or the nitrogen atom and the spirobenzopyran (indicated as ▪ in FIG. 10). This feature will be used to further optimize the chelating and release properties of spirobenzopyran based optical switches for Ca²⁺.

The absorption properties of the MC state of the Ca²⁺ switch can be tuned using different 1-nitroso-2-naphthol. For example the MC state of spironaphthoxazine (Compound 17) exhibits an absorption maximum of about 620 nm. Furthermore, the probe undergoes a far more rapid thermal reversion to the SP state (within seconds) compared to the nitrospirobenzopyran group (time constant of 370 seconds). We will investigate the photophysics of optimized Ca²⁺ switches based on spironaphthoxazine. We anticipate, based on our observations of the thermally-induced MC to SP transition that the rate of photochemistry for the spironaphthoxazine will be faster that the 11 μs reported for the spirobenzopyran group (Gorner, 2001).

Design and development of a multi-photon, pulse-probe imaging microscope for optical switches:

Concentration defined perturbations of Ca²⁺, PIP₂ and CapG will be generated by irradiating these loaded cells at defined sites with rapid pulses of 355 nm light. In addition a separately controlled port will be incorporated to deliver pulses of 532 nm light delivered by a 50 mW, frequency-doubled cw-Nd—YAG laser (Laser 2000). Separate timing controls for each laser is used to alternate the 355 nm and 532 nm pulses for optical switching of Ca²⁺ and CapG light for switches probes and conjugates for Ca²⁺, PIP₂ and CapG. These pulse-probe imaging techniques may be used for multi-photon excitation. Optical switches provide new opportunities and potential improvements over the caged approach that include reversible optical control of the levels, interactions and activities of Ca²⁺ or protein, faster perturbation kinetics, optical readout of one state of the switch, ability to control multiple switches within a single cells using the nitro and non-nitro forms of the optical switch. In addition to the studies described here, Ca²⁺ optical switches will also be used within the optical switch microscope to study the effects of generating spatially and temporally-defined Ca²⁺ transients on barbed end capping reactions in studies detailed above.

Design, Synthesis and Characterization of Thiol Reactive, Optical Switches and their Conjugates

Optical switching to modulate the interactions and activities of cytoskeleton proteins: Having demonstrated the principle and practice of optically switching specific dipolar interactions between MC and G-actin, this property may be used for optical switching of cytoskeleton-associated Ca²⁺-binding proteins as illustrated in FIG. 1B.

The difference in average energy of the MC absorption in the five G-actin conjugates (1,868 cm⁻¹) greatly exceeds that measured for the SP state in the same conjugates (504 cm⁻¹). This energy difference for the MC-G-actin interaction is comparable to that found for the interaction of G-actin with ligands and actin binding proteins (Pollard, 2003). Accordingly, the strong interaction between the MC group and specific polar groups within a protein conjugate is used to compete with the interactions underlying the binding of the MC-conjugate with a functional ligand or protein. Proteins may be engineered such that a functional interaction is perturbed for MC but not SP—therefore, optical switching between the SP and MC states would serve to reversibly modulate interactions of the spirobenzopyran conjugate.

A family of spirobenzopyran reagents may be used to project the MC dipole moment to different sites from a common attachment site where they engage in specific dipolar interactions with the protein. The origin of the remarkable differences in the dipolar interactions between these MC probes and the protein is illustrated in a qualitative study of the relative orientations of the MC and SP probes within a hypothetical protein (FIG. 5)—the orientations of the SP and MC probes were generated using the following conditions: (1), the sulfur atom on a single cysteine residue in the protein is fixed at the origin of the probe-protein reference coordinate (x,y,z). (2), the position of the spirobenzopyran molecule is constrained in this reference coordinate by forcing the atom in the aromatic ring harboring the thiol reactive group to lie on the x-axis; (3), the structures of the SP and MC probes are identical within each conjugate and are the same as those derived from crystallographic studies. Under these conditions, the direction of the MC dipole would be reversed in the protein conjugates of compounds 6 and 9, while the MC probes in the conjugates harboring compounds 3, 12 and 13 would survey a considerable volume of the protein matrix around the cysteine residue. We have chosen two simple proteins to achieve this goal. The first, parvalbumin (PA), is the simplest Ca²⁺-binding EF-hand protein (Cox et al, 1990). PA is best known as a regulator of cardiac muscle contraction but slightly different isoforms have been found to have functional roles in the actin cytoskeleton on non-muscle cells (Blum et al, 1994). PA has only 119 amino acids, a single high affinity Ca²⁺ binding site and no cysteine residues—we envision using PA as a protein-based buffer for Ca²⁺. We have analyzed the crystal structure of carp PA (Ahmed et al, 1990) and identified several non-conserved amino acids that flank the EF-hand fold—these residues will be mutated to cysteine. Analysis of crystal structure suggests that labeling of these mutants (highlighted in red) with a thiol reactive switch will position the MC probe close to the Ca²⁺-chelating groups (highlighted in yellow below), whereas the SP state will fall short of this target. By varying the geometry of the MC probe using the different thiol reactive reagents, at least one MC—PA conjugate where the MC dipole will engage in a dipolar interaction with one of these residues and thereby reducing the affinity of PA for Ca²⁺ may be generated. Macrophage and other cells microinjected with the parvalbumin optical switch will be used to modulate Ca²⁺ levels in studies similar to those outlined above for Ca²⁺ optical switches.

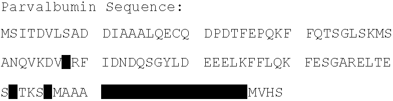

Optical perturbation of CapG function: CapG is known to bind Ca²⁺ in a similar manner to gelsolin—the Ca²⁺ ion is actually located at the interface of actin with gelsolin (McLoughlin et al, 1993). Existing cysteine residues in CapG will be replaced with Serine. 3-4 different residues in the vicinity of the Ca²⁺ binding site in CapG to cysteine may be mutated and tested to study whether the Ca²⁺- and barbed end binding activities of these mutants, or not, before proceeding to the labeling with spirobenzopyran. CapG spirobenzopyran conjugates that exhibit Ca²⁺ and barbed end binding properties in the SP but not the MC state will be identified. In a second approach to preparing a CapG optical switch, we will generate CapG mutants harboring single cysteine residues in the long α-helix of domain 1. This helix is known to interact with actin in the cleft that forms between subdomains 1 and 3. The spirobenzopyran conjugates of these CapG mutants will be tested for their ability to bind to actin in the SP but not the MC state. CapG conjugates whose functional interactions can be optically switched will be used to rapidly and reversibly perturb the barbed end capping activity of the conjugate in vitro and within CapG-null and gelsolin-null macrophage cells (Witke et al, 1995; 2001). The barbed end capping activity of the CapG optical switch will be quantified by imaging the distribution of TMR—KabC or FDE-KabC as described earlier.

Optical Switching of MC Fluorescence in Spirobenzopyran Protein Conjugates

The MC fluorescence of spirobenzopyran conjugates can be rapidly and reversibly modulated using alternate cycles of 365 nm and 546 nm light (FIGS. 22A, B; Chibisov & Gorner, 1997). Imaging techniques may be used to exploit this unique property. First spirobenzopyran conjugates as probes for speckle microscopy may be used. Second these conjugates may be used in a technique that combines PAF (Theriot & Mitchison, 1991) and FRAP (Stavreva & McNally, 2004). High fidelity, optical switching of MC-fluorescence in spirobenzopyran conjugates will be used in the following applications:
(1) FRAP/PAF: Spirobenzopyran actin conjugates as PAF/FRAP probes to study actin filament dynamics during cell protrusion
(2) Speckle microscopy: To associate the fluorescence signal observed for single to few molecules with a specific MC conjugate

The G-actin conjugate of compound 9 (FIGS. 22A, B) will be introduced into Neuro-2a, NIH 3T3 cells and macrophage cells by microinjection. These investigations will be based on our all-quartz optics Zeiss axiovert 35 microscope incorporating separate excitation ports for 365 nm and 546 nm excitation of the image field and a quartz Neofluor ×100 objective (Heidecker et al, 1996; Choidas et al, 1998). Cells will be loaded with spirobenzopyran-actin conjugate at a similar level to conventional protocols (2˜3 mg/ml) or lower in the case of speckle microscopic (Waterman-Storer & Salmon, 1997). Also this system may be investigated using 1-photon (535 nm) and 2-photon (355 nm) excitation modes to control the MC and SP states respectively.

Parallel FRAP-PAF Microscopy Using Fluorescence Optical Switches

The optical switching of MC fluorescence in spirobenzopyran protein conjugates may be used in dynamic optical imaging techniques that combine measurements of FRAP and PAF on the same actin photochromic probe in the cell same. Conducting independent measurements of the diffusion of an identical spirobenzopyran conjugate will overcome several limitations of the PAF and FRAP techniques including the issue of toxicity caused by the release of toxic photoproducts, triplet oxygen in the case of FRAP (Stavreva & McNally, 2004) and 2-nitrosobenzophenone in the case of caged fluorophores (Theriot & Mitchison, 1991). The ability to optically switch a spirobenzopyran conjugate or labeled ligand between the fluorescing MC and non-fluorescing SP state via high quantum yield photo-isomerization reactions, without the generation of toxic photoproducts, provides an internal control that is simply not possible using PAF or FRAP alone. The spirobenzopyran conjugates of G-actin will be injected into Neuro-2a, macrophage and NIH 3T3 cells to show the properties and performance of a FRAP/PAF technique and measure the diffusion rate for F-actin retrograde flow during protrusion. These measurements will be made in cells subject to optical perturbations of Ca²⁺, PIP₂, CapG perturbations will be compared to other studies (Lanni & Ware. 1984; Wang, 1985; Theriot & Mitchison, 1991). The application of multiphoton microscopy to achieve more rapid, high energy pulses of 535 nm and 355 nm light will be used to improve the temporal resolution of optical switching.

Speckle microscopic imaging of fluorescence optical switches: The fluorescence emission from a pixel element containing one or a few TMR-actin or spirobenzopyran-actin molecules will be imaged using speckle microscopy according to Waterman-Storer and Salmon et al (1997). An important factor for successful applications of the speckle microscopy technique is a requirement to show that the fluorescence emission emanating from a region of interest originates from the few to single fluorescent protein conjugates rather than an endogenous or spurious signal. A simple solution to address this issue that exploits the ability to control the fluorescence of MC in a protein conjugate using irradiation of the image field with 365 nm and 546 nm light (FIGS. 22A, B). Although the fluorescence of MC is attenuated by 546 nm light (through photoisomerization to the SP state) this reaction is not instantaneous and the preliminary data (FIGS. 22A, B) shows that high quality MC fluorescence images can be obtained during the 6 second, 546 nm-induced decay of the MC state (FIG. 22B). These measurements will be greatly enhanced when conducted in the multi-photon instrument. Because MC has a lower quantum yield compared to conventional speckle probes longer integration times may be required to obtain comparable images although this will be compensated for in MC probes by a significant reduction in photobleaching and the absence of photoproducts. However the emission properties of the other spirobenzopyran (scheme 3) that characterize conjugates may have higher quantum yields for MC emission.

Image processing: Image analysis and quantitative fluorescence microscopy (FRET and FP; Marriott et al, 1994; Yan & Marriott, 2003b) may be used for the analysis of time-series image data obtained after specific perturbations of cell Ca²⁺, PIP₂ and cytoskeleton proteins to calculate kinetic rate constants for the ensuing reactions on a pixel-by pixel basis—this allows the process or reaction to be represented and analyzed in terms of an absolute physical parameter i.e. where each pixel represents an independently determined rate constant for the reaction (Marriott et al, 1994; Yan & Marriott, 2003).

Establish the Molecular Mechanism Underlying the Regulation of Cell Protrusion

The biophotonics technologies described in above of this invention will be used to test whether the increased levels of cell Ca²⁺ and PIP₂ arising from the activation of membrane receptors regulate molecular interactions at the barbed end of the filament, actin filament dynamics and ultimately protrusion. The studies, summarized pictorially in FIG. 23 outline the roles of Ca²⁺-dependent capping, PIP₂-mediated and cofilin mediated uncapping of barbed ends in regulating actin filament dynamics and cell protrusion, the sequence, timing and localization of molecular events that lead to cell protrusion; and the local activation of signaling molecules is coupled to actin filament dynamics and the global response of cell protrusion

The dynamic, quantitative imaging studies using novel optical probes are designed to generate a spatial and temporal resolved analysis of the distributions, interactions and activities of specific signaling molecules and proteins during protrusion. Furthermore by incorporating light-directed perturbation techniques (caged and optical switches) into these imaging studies we can locally control the levels of signaling molecules and show how, or if, these signals are integrated in the lamellipodium and coupled to the global response of cell protrusion. These investigations may be performed using model macrophage cells that will cover molecular events beginning with IgG activation of membrane receptors (Serrander et al 2000) through subsequent interactions and activities of Rac1, Ca²⁺ and PIP₂, CapG, actin filament dynamics and culminating in cell protrusion.

Actin polymerization is likely to be regulated by time dependent variations in Ca²⁺ and PIP₂ levels within the lamellipodium that either promote CapG barbed end capping or dissociate the CapG complex respectively. Thus according to the model (FIG. 23; top) receptor activation is expected to trigger a modest increase in Ca²⁺ close to the membrane, which in turn activates the filament severing activity of gelsolin at this location—CapG will also be activated and bind along with CP to any free barbed ends that are generated at this location. Somewhat later, activated Rac1 increase the level of PIP₂ close to the membrane through, for example the activity of PIP5-kinase (FIG. 23, middle; Rozelle et al, 2001). PIP₂ binds to and dissociates CapG, gelsolin and CP bound to the barbed end at this site. The free barbed ends will then rapidly polymerize though dendritic growth (Pollard , 2003) causing cell protrusion (FIG. 18). Cofilin may work in concert with PIP₂ to create barbed ends, or through a Ca²⁺ and PIP₂ independent fashion earlier in the pathway (Ghosh et al, 2004). Finally PIP₂ is hydrolyzed by phospholipase C generating Diacylglycerol and inositol triphosphate, which stimulates the further release of Ca²⁺ by IP₃ and calcium induced calcium release (CICR) from intracellular stores. This is a slower process and leads to a sustained increase in cell Ca²⁺. We expect that the timing of the breakdown of PIP₂ causes the cessation of actin polymerization due to the increased amount of active CapG, which will bind to free barbed ends. The loss of PIP₂ will significantly reduce the amount of barbed end uncapping according to the model.

These studies may be performed within CapG-null and CapG-Gelsolin double null macrophage cells from the Witke lab (Witke et al, 2001) and Neuro-2a neuroblastoma (ATTC). Many of the transients invoked in our model will be generated optically through light directed activation of caged precursors and/or optical switches of Ca²⁺, PIP₂, cofilin and CapG previously loaded into cells. Our microscope workstation is set up for simultaneous uncaging and fluorescence imaging (Marriott & Heidecker, 1996) and will be used to generate rapid and localized perturbations within Neuro-2a and macrophage cells. Analysis of accompanying molecular events at the barbed end will be quantified by time-resolved FRET imaging of appropriately labeled probes (see below; actin, TMR-KabC and CapG; Heidecker et al, 1995) using speckle and/or 2-photon confocal microscopies with Peter So.

Fluorescent Probes to Quantify Binding at the Barbed-End of the Actin Filament:

Fluorescent KabC probes provide unrivalled contrast for imaging molecular events at the barbed end as shown by their actin binding specificity in Tanaka et al (2003) and Klenchin et al (2003). Quantitative fluorescence imaging of the membrane permeable fluorescein-KabC, shown in FIG. 19, and TMR—KabC will be used to map the distributions of free and barbed end bound KabC-G-actin during ligand or light-directed activation of protrusion within protrusive cells. An increase the intensity of the fluorescence of KabC probes at sites close to the site of Ca²⁺, PIP₂ uncaging, or IgG activation of membrane receptor (Serrander et al 2000) would indicate an increase in the number of free barbed ends at that site. A cell loading protocol described in Choidas et al (1998) and Tanaka et al (2003) will be used to ensure that these probes function as spectators, and not actors, in the physiological drama of cell protrusion. The new TMR—KabC will also be used as an acceptor probe for GFP-actin in FRET based imaging of capping and uncapping events at the barbed-end of the filament (Heidecker et al, 1995; Yan & Marriott, 2003a). In addition FRET between GFP-actin (Choidas et al, 1998) and TMR-CapG will be used to independently monitor events at the barbed end of the filament in response to ligand, or optical activation of second messengers and CapG in cells.

The Role of CapG in Regulating Actin Filament Dynamics During Cell Protrusion.

The approach involves imaging the distributions and interactions between CapG and the barbed end using microinjected or genetically encoded fluorescent conjugates of CapG, actin and KabC. Specifically, we will use: (1), GFP-fusions of actin (Choidas et al, 1998) and CapG; (2), functional fluorescent CapG conjugates (fluorescein, TMR and IC5-CapG microinjected into macrophage cells; (3), FDE- and TMR-labeled KabC (preliminary achievements). Capping activity in macrophage cells will be mapped using either the fluorescence emission from: (a), FDE- or TMR—KabC; (b), microinjected TMR—CapG; (c), GFP—CapG. Changes in the distribution of actin filaments will be imaged using either GFP-actin in the case of (a) and (c) or microinjected IC3-actin in the case of (b). This combination of probes will also serve to image interactions between the barbed end and CapG using sensitized FRET emission between pairs of appropriately labeled proteins. Simultaneous acquisition of the phase contrast image will allow us to correlate molecular events associated with the uncapping of actin filaments to the explosive polymerization of F-actin and the associated protrusion of the lamellipodium. The role of CapG in regulating actin polymerization in cell protrusion will be further advanced in studies using light-directed, in vivo perturbation of Ca²⁺ , PIP₂ and CapG from their caged precursors, or optical switches in the case of Ca²⁺ and CapG (described above), that are loaded into cells by microinjection or as membrane permeable probes. These goals will be met through the following studies:
(1) Mapping changes in the distributions of IC3-CapG and GFP-actin (Choidas et al, 1998) during light-directed activation of caged Ca²⁺. Image based measurements of FRET between (a) GFP-actin and IC3-CapG and (b), IC3-actin and FDE-KabC or TMR—KabC or IC5-KabC will serve to map the distributions of actin filaments and free barbed-ends before and after photoactivation.
(2) Correlating changes in the dynamics of the CapG-actin filament complex with changes in the organization of actin (GFP-actin images) and cell protrusion (phase) in response to IgG-mediated activation of membrane receptors that lead to cell protrusion.

The criteria for using GFP-actin as a probe of actin structure and dynamics (Choidas et al, 1998) have been discussed. The purified CapG and actin conjugates will also be tested for their binding activity using in vitro assays based on FRET between the donor and acceptor conjugates and the effect of the CapG conjugate on the fluorescence of Prodan-actin (FIG. 20B). However, GFP has limitations and it effects the activity of ABPs (Choidas et al, 1998). Accordingly, the inventors have established cell loading conditions where fluorescein- and TMR—KabC function as a probe of barbed end capping and actin filament dynamics without impairing other functions of the actin cytoskeleton (Tanaka et al, 2003). TMR and fluorescein conjugates of CapG will be characterized using the assays detailed in Marriott et al (1998) and shown in part in FIGS. 20 and 21. Light directed activation of caged proteins (Marriott et al, 1992) may be used as controls to ensure that the observed effects of the irradiation are solely due to the activation of the second messenger or protein and not due to other factors e.g. photoproducts detailed in Roy et al (2001).

Role of Cell Ca²⁺ in Regulating Actin Filament Dynamics During Cell Protrusion

Localized, light directed generation of concentration jumps of Ca²⁺ from commercially available caged NP-EGTA and DM-Nitrophen and optical switching of Ca²⁺ using the probes developed as shown above and from the optical switch conjugate of parvalbumin may be used to mimic the effect of receptor-mediated rises in cell Ca²⁺ within macrophage cells undergoing cell protrusion. This approach will allow us to test whether an increase in level of Ca²⁺ at the plasma membrane at the barbed end by CapG (FIG. 23 upper) that blocks actin polymerization and membrane protrusion. Higher levels of Ca²⁺ will also be generated using this approach in order to test the wheather sustained and high levels of Ca²⁺ and lower levels of PIP₂ serve to suppress cell protrusion by overwhelming free barbed ends with Ca²⁺ bound CapG and gelsolin. Specifically, 1- and 2-photon, FRET and speckle imaging modalities will be conducted to map and quantify the effects of increasing Ca²⁺ from caged precursors or optical switches at defined sites in the lamellipodium on: (a), the number and distribution of barbed-ends; (b), changes in the dynamics of actin filaments at the site of photoactivation; (c), changes in the protrusive activity of the plasma membrane. These studies will be conducted using CapG-null and CapG/Gelsolin double-null cells from transgenic mice received from the Witke laboratory (Witke et al, 2001) and in Neuro-2a cells. The ability to rapidly and reversibly switch Ca²⁺ with optical switch chelates will provide a more accurate picture to evaluate the effects of Ca²⁺ transients within the lamellipodium. Most protrusion will occur when Ca²⁺ and PIP2 levels are high (FIG. 18) but that a decrease in PIP₂ or a sustained increase in Ca²⁺ caused by CICR will activate a substantial fraction of the barbed end capping protein in a cell and thereby overwhelm the free barbed ends and thereby halt cell protrusion.

Ca²⁺-independent generation of free barbed ends: Caged cofilin conjugates may be used together with the specific fluorescent KabC probes to image and quantify free barbed ends that result from the activation of cofilin (FIG. 23 lower). These studies will provide an independent analysis of the far reaching conclusions drawn by the Ghosh et al (2004) on the role of cofilin in cell protrusion and motility. The Condeelis model (Ghosh et al, 2004) could explain the origin of the numerous barbed ends generated during cell motility that cannot be accounted for by the severing activity of gelsolin (FIG. 23). This question will be addressed by quantifying the number of barbed ends generated by the activation of caged cofilin in the absence and presence of optically generated Ca²⁺ transients (Caged forms and optical switches). This comparative analysis of will be realized through the unique barbed end binding properties of the fluorescent KabC probes. As necessary the caged cofilin may also be prepared as described in Ghosh et al (2004).

Specifically these experiments involve: Mapping changes in the distributions of IC3-CapG and GFP-actin (Choidas et al, 1998) during light-directed activation of caged cofilin. Image based measurements of FRET between (a) GFP-actin and IC3-CapG and (b), IC3-actin and FDE-KabC or TMR—KabC or IC5-KabC will serve to map the distributions of actin filaments and free barbed-ends before and after activation of cofilin. These experiments will be performed in wild type and gelsolin-null macrophage cells in at low and elevated levels of Ca²⁺ and PIP₂ by uncaging DM-Nitrophen or NP-EGTA and caged PIP₂, or else using spirobenzopyran optical switches for chelating Ca²⁺ and parvalbumin (Aims 1 and 2) will be quantified using Fluo-3 and X—Rhod-1 probes loaded into cells by microinjection or as AM-esters.

Establish the Role of PIP₂ in Uncapping Barbed Ends During Cell Protrusion

Light directed concentration jumps of PIP₂ from caged PIP₂ may be used to mimic the effect of receptor-mediated signaling pathways in macrophage cells. This approach studies whether an increase in PIP₂ close to the plasma membrane of the leading edge dissociates CapG from the barbed end of the actin filament triggering polymerization and membrane protrusion (FIG. 23 middle). This may be done with imaging studies that are designed to map and quantify the effects of increasing PIP₂ at defined sites in the lamellipodium on: (a), the number and distribution of barbed-ends; (b), changes in the dynamics of actin filaments at the site of photo-activation; (c), changes in the protrusive activity of the plasma membrane. Modulation of molecular interactions at the barbed end and associated changes in actin dynamics will be probed by FRET based analysis of appropriately labeled KabC CapG and actin as outlined above. Controlled photo-activation of Rac1 (see below) will be used to study the role of Rac1-mediated generation of PIP₂ during cell protrusion (FIG. 23 middle). Image based measurements of FRET between (a) GFP-actin and TMR—CapG and (b), IC3-actin and FDE-KabC or IC5-KabC will serve as suitable FRET systems to analyze the distributions of actin filaments, free and CapG capped barbed-ends before and after photoactivation of caged PIP₂ in the absence and presence of calcium.

Caged PIP₂: Surprisingly caged PIP₂ has not been described in the literature yet this would be a most interesting and useful probe to study PIP₂ regulation of essential processes including motility and synaptic signaling. For this, a caged PIP₂ derivative will be synthesized through the reaction of 1-(2-nitrophenyl)diazoethane phosphate with PIP₂ (Calbiochem) in chloroform/water mixture as described and routinely practiced (Walker et al, 1989).

Caged Rac1: Light directed activation of caged Rac1 will be used to generate localized jumps in PIP₂ mediated and to correlate this event with barbed-end uncapping. Constitutively active Racl (G12V) and dominant negative mutants of human Racl have been prepared in >30 mg quantities as described in Faix et al (2001). Caged Rac1 will be prepared using three different methods. (a), Cysteine-189 of Rac1 will be modified using our thiol reactive caging group (Marriott & Heidecker, 1996)—membrane anchoring through this cysteine residue is absolutely required for Rac1 function. Cysteine 189 and a limited number (3-4) other cysteine residues will be labeled with bromomethyl-3,4-dimethoxynitrobenzene (Marriott & Heidecker, 1996). The activity of the constitutively active Rac1 (control) and caged Rac1 conjugate will be measured using our in vitro DGAP1 binding assay (Faix et al, 2001) or by membrane ruffling activity when microinjected into serum deprived cells (Ridley, 1995; Ridley & Hall, 1992) before and after irradiation with a pulse of uv light (Roy et al, 2001); (b), Constitutively active Rac1 will be modified at one or two lysine residues with the photo-cleavable reagent BNBA-NHS (Marriott et al, 1992). The Rac1 conjugate will then be crosslinked to a TMR-labeled, thiolated dextran (Otti et al, 1998) in order to physically block binding sites on the Rac1 molecule. The activity of the unmodified, caged and uncaged Rac1-dextran complex will be determined as described above. Uncaging experiments in cells will be subject to the same controls described in Roy et al (2001). PIP₂ may also be quantified using a fluorescent peptide indicator of PIP₂ described by Tuominen et al (1999).

The Signaling Pathway Leading to Actin Mediated Cell Protrusion

The signaling pathway leading to cell protrusion by correlating changes may be dissected in the spatial and temporal distributions of these molecules and ions and their interactions to the generation of free barbed ends, the polymerization of actin filaments and cell protrusion as outlined in FIG. 23 (top, middle and bottom). The sequence and timing of molecular events of protrusion in gelsolin/CapG double null macrophage cells beginning with IgG activation of membrane receptors to using correlative, 1- and 2-photon, time-resolved, multi-mode fluorescence imaging microscopy with the previously described fluorescent and caged conjugates of Rac1, CapG, actin, PIP₂ (Echelon) and KabC as well as Ca²⁺ indicator probes e.g. X—Rhod-1 or Fluo-3, may be established. These studies will include the application of light directed activation of caged second messengers and proteins outlined above, to short circuit the receptor activated signaling pathway. Furthermore, optical switches for Ca²⁺ and CapG will be employed, which represents a new and improved approach for light directed perturbation of specific proteins and ions. By imaging the kinetics and robustness of cell protrusion in response to rapid and spatially defined perturbations of CapG, cofilin, Rac1, PIP₂ and Ca²⁺ from their caged or optical switch precursors, an accurate temporal and spatially resolved map of the IgG receptor-activated signaling pathway that leads to cell protrusion will be generated. By varying the amount of second messenger and signaling protein using different energy pulses of uncaging or optical switching light, local activities and interactions of signaling ions and molecules are integrated in the lamellipodium and coupled to the regulation of actin polymerization may be determined. Specifically, these studies will involve:
(1) Mapping changes in the distribution of IC3-CapG and actin (GFP-actin fluorescence) during receptor mediated activation of cell motility. Image based measurements of: (i), FRET between GFP-actin and IC3-CapG (ii), and TMR— and FDE-KabC will serve as an quantitative probe of the distribution of capped barbed-filament ends at the leading edge
(2) Correlating changes in the dynamics of the CapG-actin filament complex (FRET signal) with changes in free-barbed ends, actin filament dynamics and protrusion of the leading edge (phase) at the leading edge in response to local, light-directed perturbations of caged Ca²⁺ and Ca²⁺-optical switches
(3) Correlating changes in the dynamics of the CapG-actin complex with changes in free-barbed ends, actin filament dynamics and cell protrusion (phase) in response to activation of a caged Rac1.
(4) Correlating changes in the dynamics of the CapG-actin filament complex in free-barbed ends with actin filament dynamics and protrusion (phase) in response to light directed activation of caged cofilin in the absence and at elevated levels of Ca²⁺ and PIP₂.
(5) Correlating changes in the dynamics of the CapG-actin filament complex (FRET signal) with changes in free-barbed ends, actin filament dynamics and protrusion of the leading edge (phase) at the leading edge in response to local, light-directed perturbations of caged PIP₂.
(6) Correlating changes in the dynamics of the CapG-actin filament complex (FRET signal) with changes in free-barbed ends, actin filament dynamics and protrusion of the leading edge (phase) at the leading edge in response to local optical perturbations of caged CapG and/or a CapG optical switch
(7) Correlating changes in the dynamics of the CapG-actin filament complex (FRET signal) with changes in free-barbed ends, actin filament dynamics and protrusion of the leading edge (phase) at the leading edge in response to IgG-mediated receptor activation of membrane ruffling.

The sequence of events in receptor mediated signaling of cell motility is likely to be in the following order: 1. IgG Receptor activation; 2. Rac1 activation; 3. Activation of cofilin; 4. Increase in Ca²⁺; 5. Increase in PIP₂; 6. Uncapping actin filament barbed-ends; 7. Polymerization of actin filaments in the lamellipodium; 8. Cell protrusion. Furthermore, these events may be confined to the plasma membrane.

Specific Methods

Instrumentation. ¹H NMR spectra were measured on a Brucker Ac 300 MHz; mass spectra were carried out on a Micromass AutoSpec for El, a Micromass LCT for ESI, or a Bruker REFLEX II for MALDI. Absorption spectra were recorded on a Hewlett-Packard 82152 diode array spectrophotometer or a Shimadzu 1601PC instrument. Fluorescence spectroscopy was performed on an SLM-AB2 instrument (Thermoelectron, Madison, Wis.). Light-directed optical switching is achieved by irradiating the sample (120-1000 μL) with the 365 nm or 546 nm lines of a 100 W Hg-arc lamp (Zeiss).

Live cell microscopy: The multi-model microscope workstation for imaging transmission and fluorescence images of living cells (Choidas et al, 1998) incorporates a 100 W Hg-arc lamp allowing for simultaneous fluorescence and flash photolysis of caged compounds. A double-view dichroic mirror assembly is used that separately projects the GFP and TMR images onto a single camera (Heidecker et al, 1995). This technique is particularly useful for recording molecular interactions between GFP-actin and IC3-protein conjugates by real-time imaging of GFP-fluorescence and TMR-sensitized emission. Cells are maintained at 37° in a perfusion chamber (Choidas et al, 1998).

Cloning. All molecular biology methods used in this proposal are routinely used in the PI's laboratory (see papers by Prassler et al, 1998; Stocker et al, 1999; Westphal et al, 1997; Faix et al, 2001). Cloning and gene expression: Genes encoding CapG, Gelsolin, Rac1 and cofilin are cloned from a mouse brain cDNA library (Stratagene) and the clone for mouse β-parvalbumin obtained from ATCC. The genes are cloned into expression vectors and expressed following induction with IPTG. Gelsolin, cofilin and CapG genes are amplified by PCR using a mouse brain cDNA library (Invitrogen) as a template and gene specific primers and cloned in the HindIII, BamHI site of the pQE30 vector that has an N-terminal His-tag. The M15 bacterial strain is used to express the genes.

Protein purification: A 1.6 L culture is induced with 1 mM IPTG at 30° or 37° for 5 to 6 h. The soluble proteins are purified using Ni—NTA (Qiagen manual). For example the gene encoding CapG is cloned from a mouse cDNA library (Invitrogen) and expressed and purified as a soluble His-tagged protein in E. coli using an NTA-sepharose column. About 50 mg of pure CapG is purified from a 600 ml culture. Rabbit muscle G-actin is purified according to Marriott (1994). The concentration of G-actin was determined by absorption using an extinction coefficient of 3400 M⁻¹ cm⁻¹ at 290 nm¹⁴. The purity and activity of actin is determined by SDS-PAGE and polymerization assays.

Antibodies: New polyclonal antibodies have been developed against Gelsolin, cofilin, actin, Rac1 and CapG that work well in Western blots. A new polyclonal antibody has also been developed against the NVOC group and should prove to be useful to quantify uncaging reactions.

Protein labeling: Actin, CapG, Gelsolin and cofilin are labeled with the thiol and amino reactive donor dyes: Acrylodan, 5′-TMR-maleimide (Molecular Probes) or IC3-maleimide, IC5-maleimide and IC3-NHS (Dojindo) and thiol reactive spirobenzopyrans using standard protocols in the inventors' laboratory (Marriott et al, 1988). All fluorescent conjugates are analyzed for labeling ratio (<1:1) and binding to F-actin. The activity of the conjugates is assessed using Prodan-actin assays. The extinction coefficient for SP is taken as 35,000 M⁻¹cm⁻¹ at 350 nm and 52,000 M⁻¹cm⁻¹ at 530 nm for MC.

Cells: The molecular basis of actin-based protrusion will be studied using several model cell lines including macrophage cells. These will be isolated from wild type mice, CapG-null mice, Gelsolin-null mice and CapG/Gelsolin double null mice may be used. Macrophage cells exhibit a dramatic IgG-mediated ruffling that is suppressed in CapG-null mice. This activity is restored after microinjecting CapG. Neuro-2a cells (Rosner et al, 1995) are obtained from ATTC.

Furthermore, the compounds and a method of using the photochromic probes of the present invention may have other applications aside from use calcium ion chelating probes. Additionally, it would be apparent to one of ordinary skill in the art to alter the methods and compositions which have been described herein in the preferred embodiment. Such alterations include altering the starting compound and making substitutions, without departing from the spirit of the invention, or altering the positional chemistry, stereochemistry and conformations of the compounds. Further alterations include creating salts of these compounds by techniques and methods known to one of ordinary skill in the art. Thus, although the invention has been herein shown and described in what is perceived to be the most practical and preferred embodiments, it is to be understood that the invention is not intended to be limited to the specific embodiments set forth above. Rather, it is recognized that modifications may be made by one of skill in the art of the invention without departing from the spirit or intent of the invention and, therefore, the invention is to be taken as including all reasonable equivalents to the subject matter of the appended claims.

### REFERENCES

(1) Zhang, J., Campbell, R. E., Ting, A. Y., and Tsien, R. Y. (2002) Creating New Fluorescent Probes for Cell Biology. Nat. Rev. Mol. Cell Bio. 3, 906-918.
(2) Yan, Y., and Marriott, G. (2003) Analysis of protein interactions using fluorescence technologies. Curr. Opin. Chem. Biol. 7, 635-640.
(3) Roy, P., Rajfur, Z., Jones, D., Marriott, G., Loew, L., and Jacobson, K. (2001) Local photorelease of caged thymosin beta4 in locomoting keratocytes causes cell turning. J. Cell Biology 153, 1035-1048.
(4) Miyawaki, A., and Tsien, R. Y. Monitoring protein conformations and interactions by fluorescence resonance energy transfer between mutants of green fluorescent protein. Method. Enzymol. 327, 472-500.
(5) Marriott, G., Miyata, H., and Kinosita, K., Jr. (1992) Photomodulation of the nucleating activity of a photocleavable crosslinked actin dimer. Biochemistry Int. 26,943-951.
(6) Marriott, G., and Heidecker, M. (1996) Light-directed generation of the actin-activated ATPase activity of caged heavy meromyosin. Biochemistry35, 3170-3174.
(7) Giordano, L., Jovin, T. M., Irie, M., and Jares-Erijman, E. A. (2002) Diheteroarylethenes as thermally stable photoswitchable acceptors in photochromic fluorescence resonance energy transfer (pcFRET). J. Am. Chem. Soc. 124, 7481-7489.
(8) Medintz, I. L., Trammell, S. A., Mattoussi, H., and Mauro, J. M. (2004) Reversible modulation of quantum dot photoluminescence using a protein-bound photochromic fluorescence resonance energy transfer acceptor. J. Am. Chem. Soc. 126, 30-31.
(9) Dale, R. E., Eisinger, J., and Blumberg, W. E. (1979) The orientational freedom of molecular probes. The orientation factor in intramolecular energy transfer. Biophysical J. 26, 161-193.
(10) Inouye, M. (1994) Spiropyran derivatives as multifunctional artificial receptors for biologically important species. Mol. Cryst. Liq. Cryst. A 246,169-172.
(11) Willner, I., Rubin, S., Wonner, J., Effenberger, F., and Baeuerle, P. (1992) Photoswitchable binding of substrates to proteins: photoregulated binding of a-D-mannopyranose to concanavalin A modified by a thiophenefulgide dye. J. Am. Chem. Soc. 114, 3150-3151.
(12) Angelini, N., Corrias, B., Fissi, A., Pieroni, O., and Lenci, F. (1998) Photochromic polypeptides as synthetic models of biological photoreceptors: a spectroscopic study. Biophysical J. 74, 2601-2610.
(13) Song, L., Jares-Erijman, E. A., and Jovin, T. M. (2002) A photochromic acceptor as a reversible light-driven switch in fluorescence resonance energy transfer (FRET). J. Photoch. Photobio. A 150, 177-185.
(14) Marriott, G. (1994) Caged protein conjugates and light-directed generation of protein activity: preparation, photoactivation, and spectroscopic characterization of caged G-actin conjugates. Biochemistry 33, 9092-9097.
(15) Gorner, H. (2001) Photochromism of nitrospiropyrans: effects of structure, solvent and temperature. Phys. Chem. Chem. Phys. 3, 416-423.
(16) Inouye, M., Ueno, M., Tsuchiya, K., Nakayama, N., Konishi, T., and Kitao, T. (1992) Alkali-metal cation recognition induced isomerization of spirobenzopyrans and spironaphthoxazins possessing a crown ring as a recognition site: multifunctional artificial receptors. J. Org. Chem. 57, 5377-5383.
(17) Tanaka, J., Yan, Y., Choi, J., Bai, J., Klenchin, V. A., Rayment, I., and Marriott, G. (2003) Biomolecular mimicry in the actin cytoskeleton: Mechanisms underlying the cytotoxicity of kabiramide C and related macrolides. P. Natl. Acad. Sci. U.S.A. 100, 13851-13856.
(18) Raymo Francisco, M., Giordani, S., White Andrew, J. P., and Williams David, J. (2003) Digital processing with a three-state molecular switch. J. Org. Chem. 68, 4158-4169.
(19) Walker, M. A. (1995) A High Yielding Synthesis of N-Alkyl Maleimides Using a Novel Modification of the Mitsunobu Reaction. J. Org. Chem. 60, 5352-5355.
(20) Weber, G., and Farris, F. J. (1979) Synthesis and spectral properties of a hydrophobic fluorescent probe: 6-propionyl-2-(dimethylamino)naphthalene. Biochemistry 18, 3075-3078.
(21) Bletz, M., Pfeifer-Fukumura, U., Kolb, U., and Baumann, W. (2002) Ground- and First-Excited-Singlet-State Electric Dipole Moments of Some Photochromic Spirobenzopyrans in Their Spiropyran and Merocyanine Form. J. Phys. Chem. A 106, 2232-2236.
(22) Chibisov, A. K., and Goerner, H. (1997) Photoprocesses in Spiropyran-Derived Merocyanines. J. Phys. Chem. A 101, 4305-4312.
(23) Marriott, G., Zechel, K., and Jovin, T. M. (1988) Spectroscopic and functional characterization of an environmentally sensitive fluorescent actin conjugate. Biochemistry 27, 6214-6220.
(24) Tanaka, J., Yan, Y., Choi, J., Bai, J., Klenchin, V. A., Rayment, I., and Marriott, G. (2003) Biomolecular mimicry in the actin cytoskeleton: Mechanisms underlying the cytotoxicity of kabiramide C and related macrolides. P. Natl. Acad. Sci. U.S.A. 100, 13851-13856.
(25) Kouyama, T., and Mihashi, K. (1981) Fluorimetry study of N-(1-pyrenyl)iodoacetamide-labeled F-actin. Local structural change of actin protomer both on polymerization and on binding of heavy meromyosin. Eur. J. Biochem. 114, 33-38.
(26) Macgregor, R. B., and Weber, G. (1986) Estimation of the polarity of the protein interior by optical spectroscopy. Nature 319, 70-73.
(27) Heidecker, M., Yan-Marriott, Y., and Marriott, G. (1995) Proximity relationships and structural dynamics of the phalloidin binding site of actin filaments in solution and on single actin filaments on heavy meromyosin. Biochemistry 34, 11017-11025.
(28) Clapp A R, Medintz I L, Mauro J M, Fisher B R, Bawendi M G, Mattoussi H. Fluorescence resonance energy transfer between quantum dot donors and dye-labeled protein acceptors. J Am Chem Soc.126, 301-10

What is claimed is:

1. A compound or a derivative selected from the group consisting of: 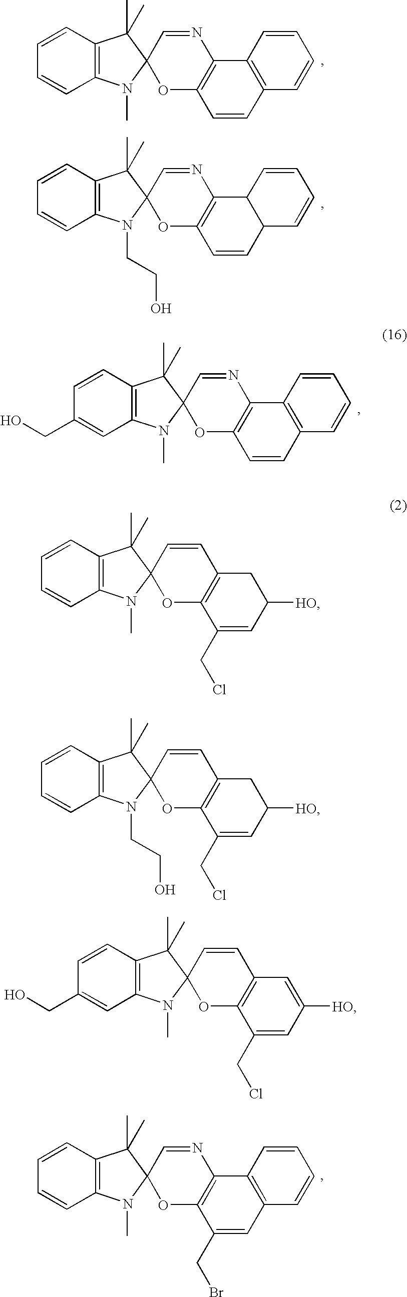 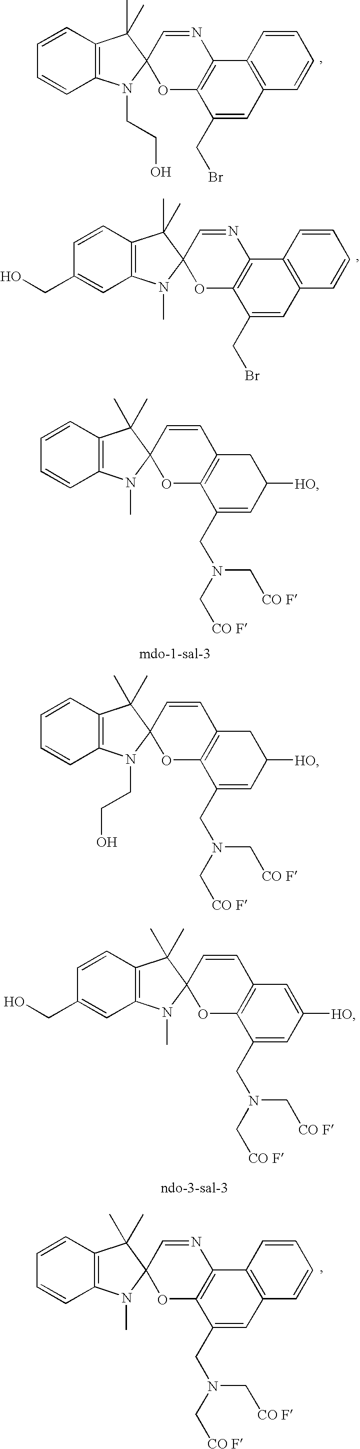 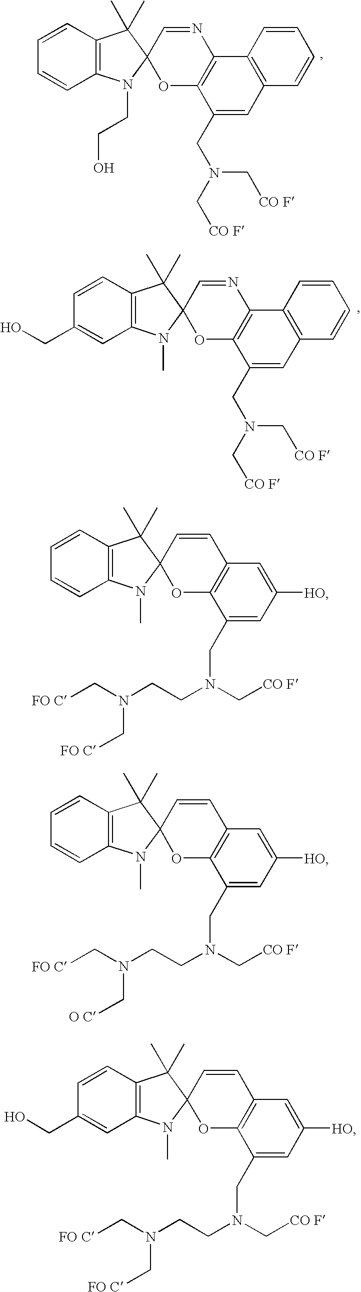 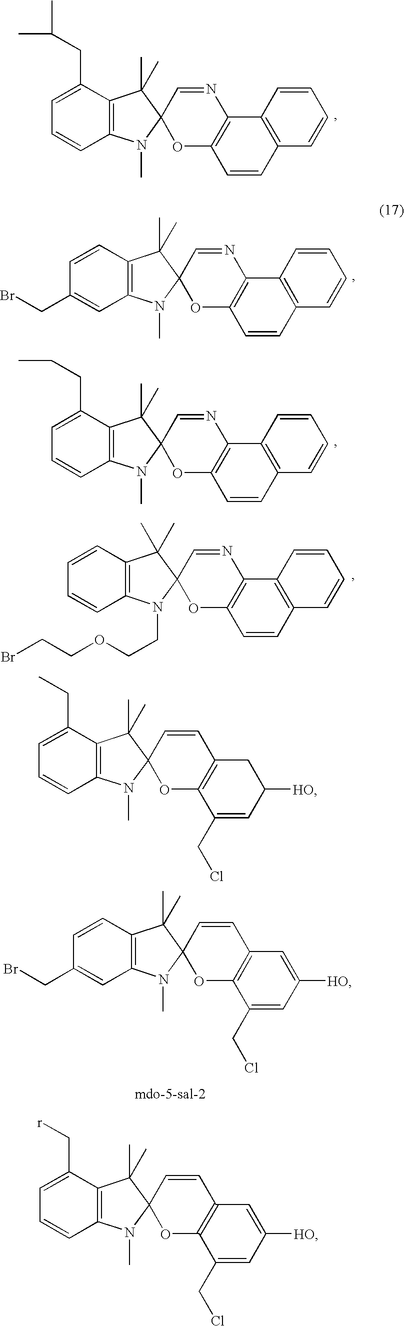 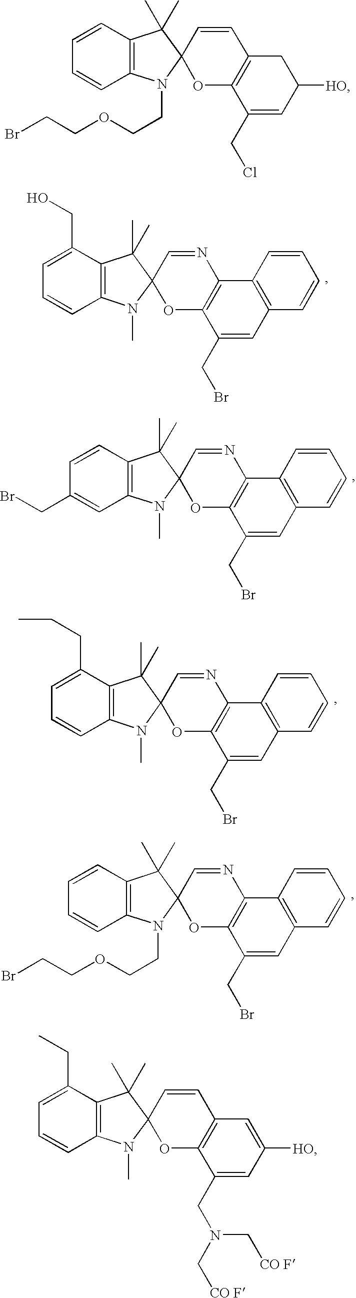 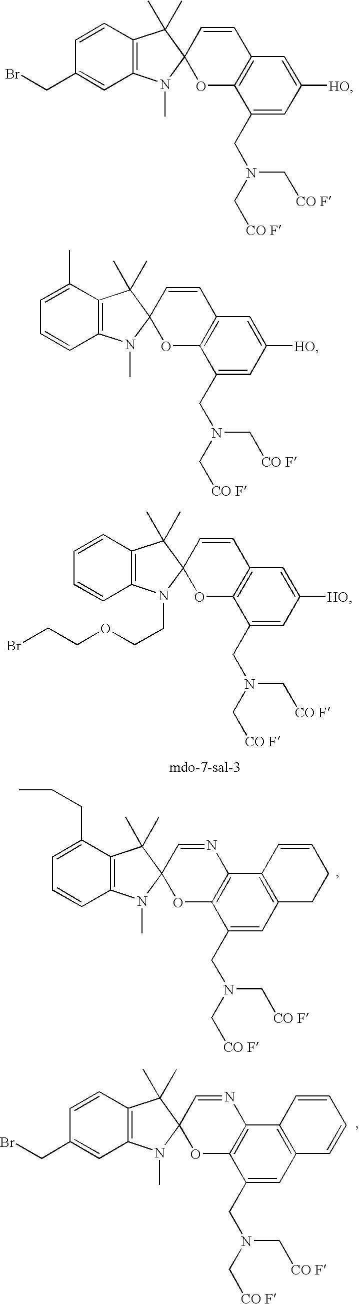 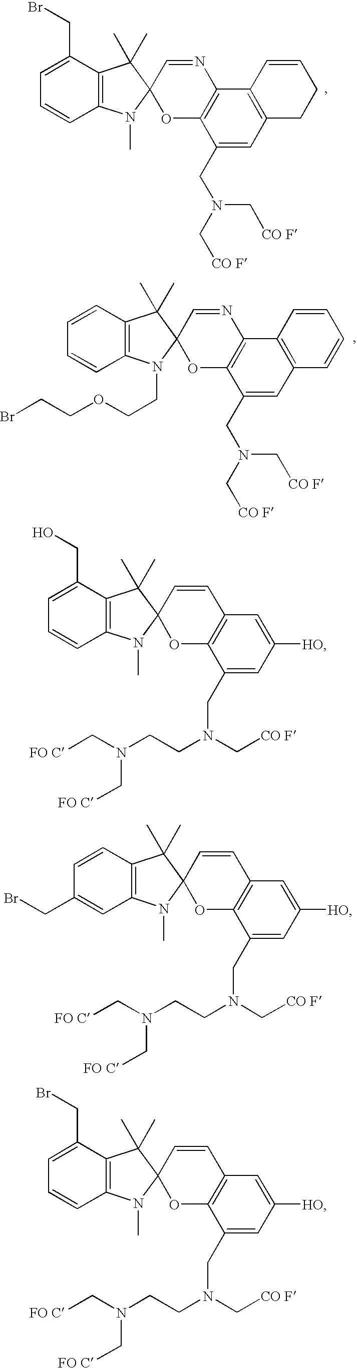 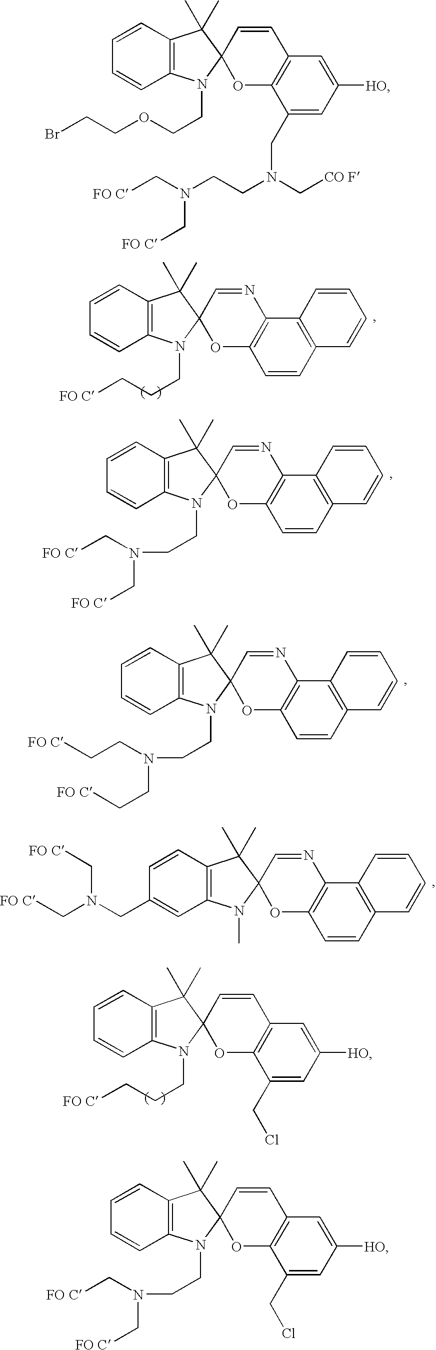 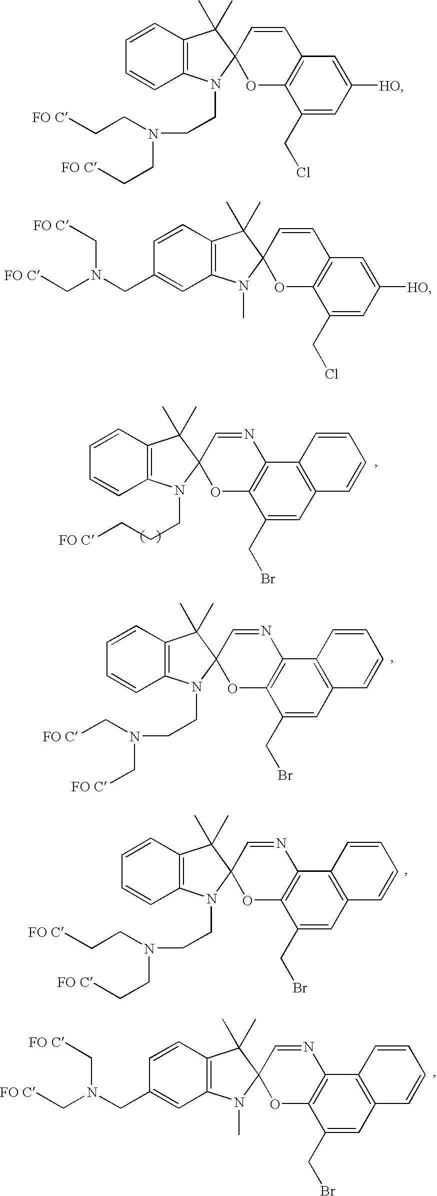 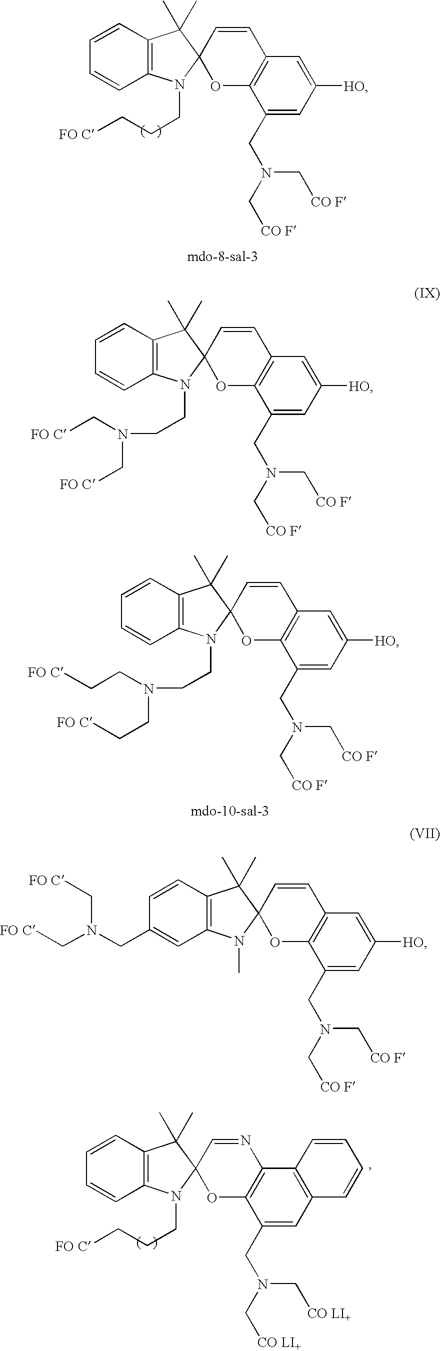 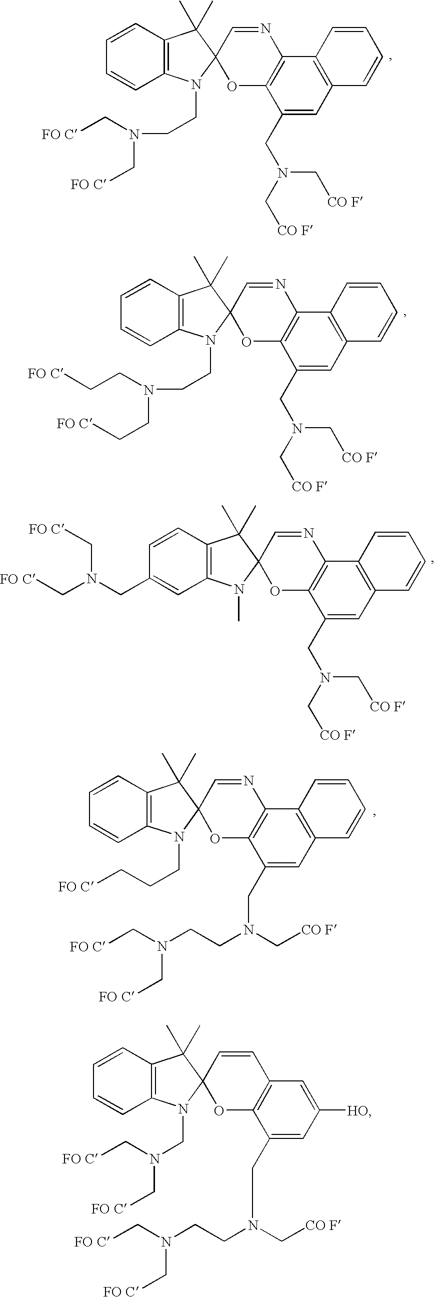 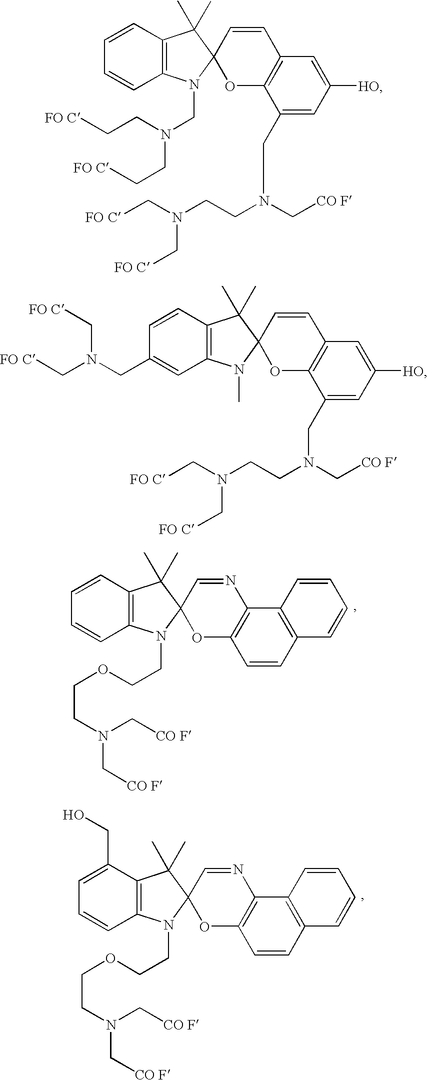 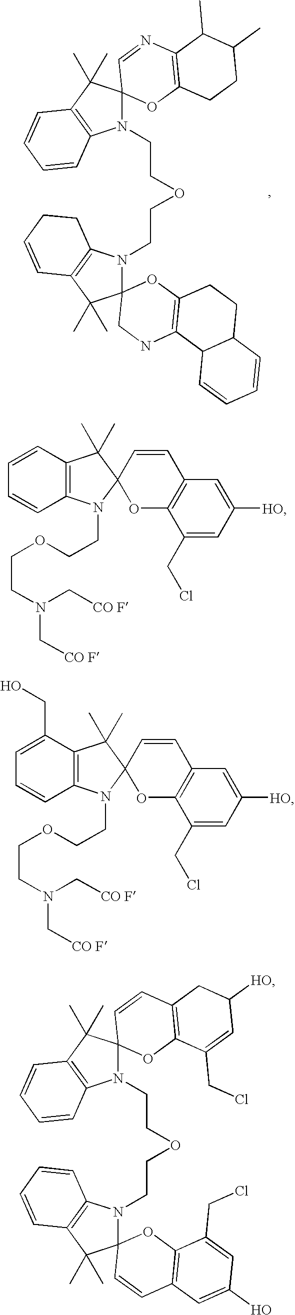 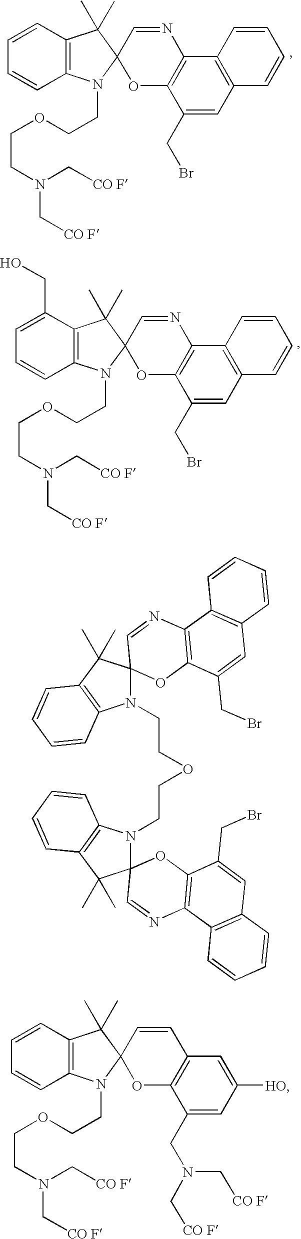 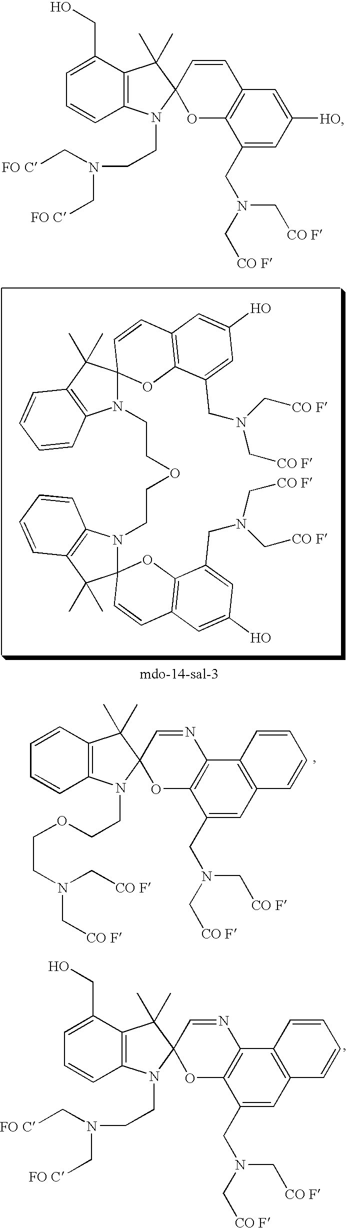 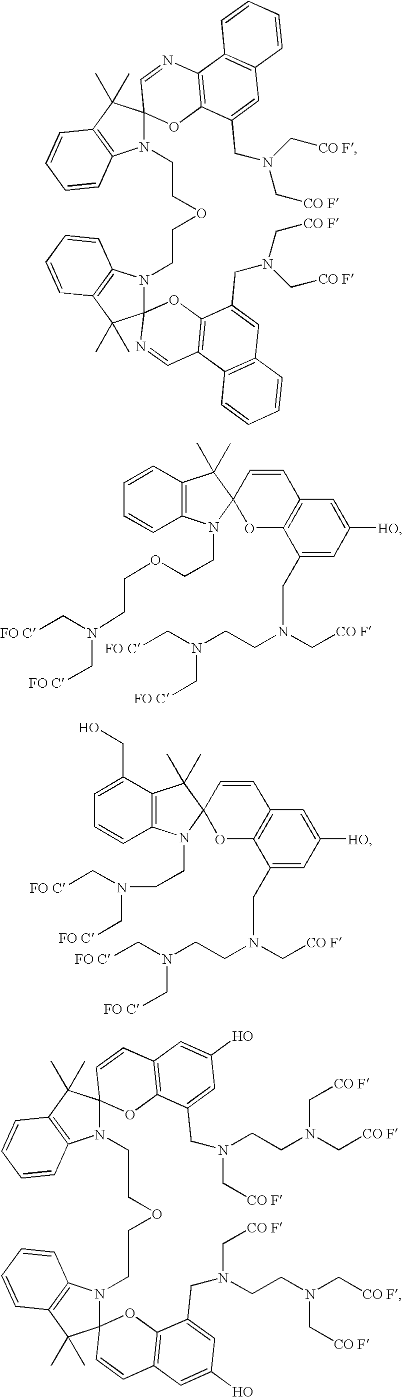 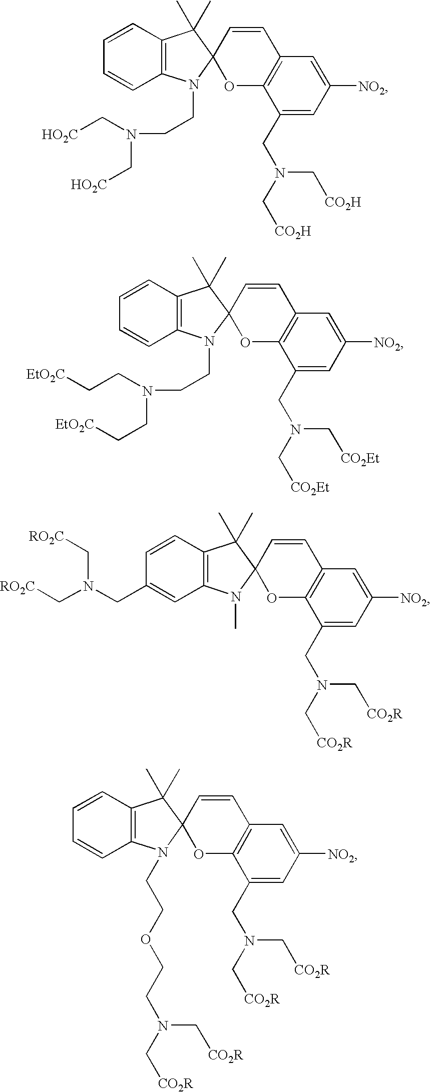 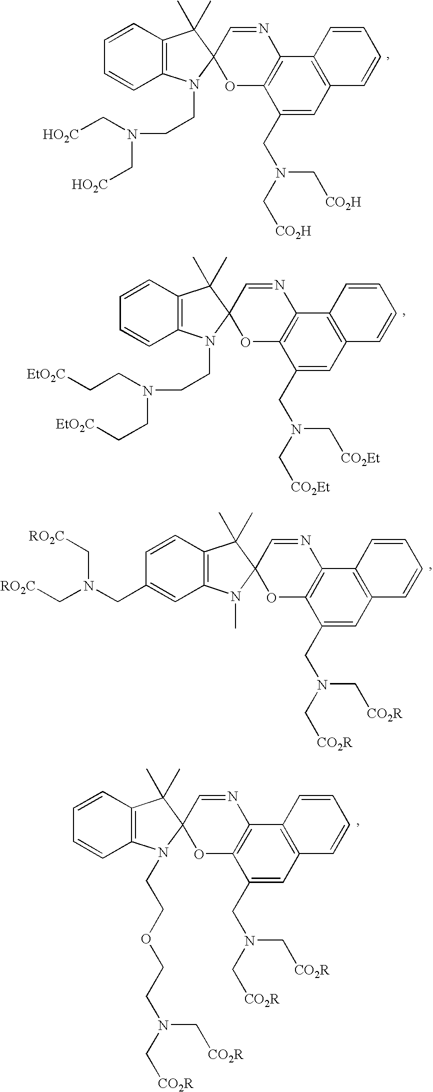 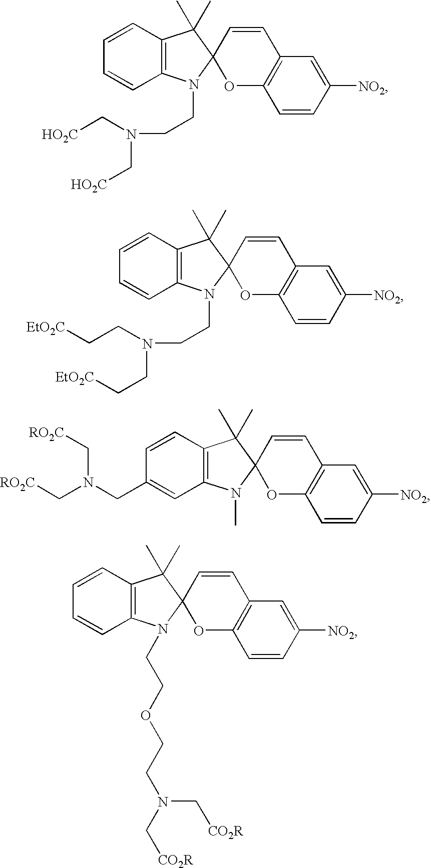 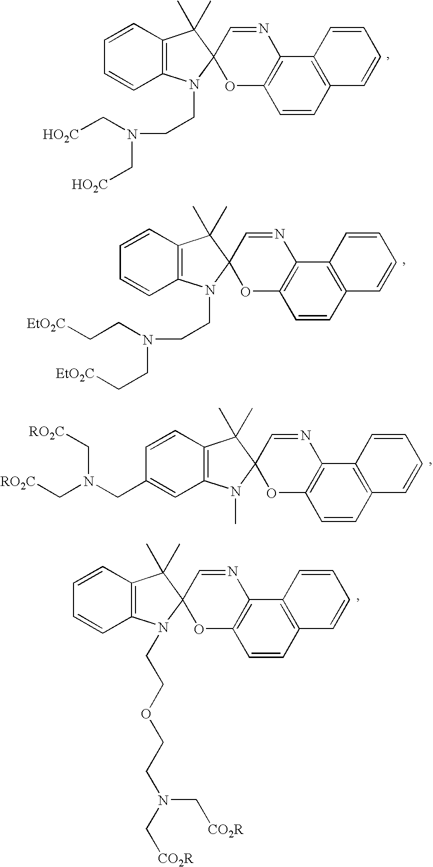 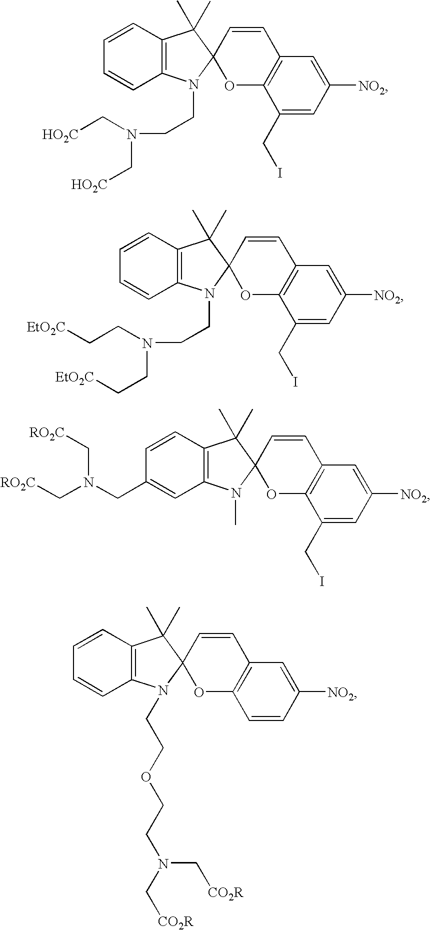 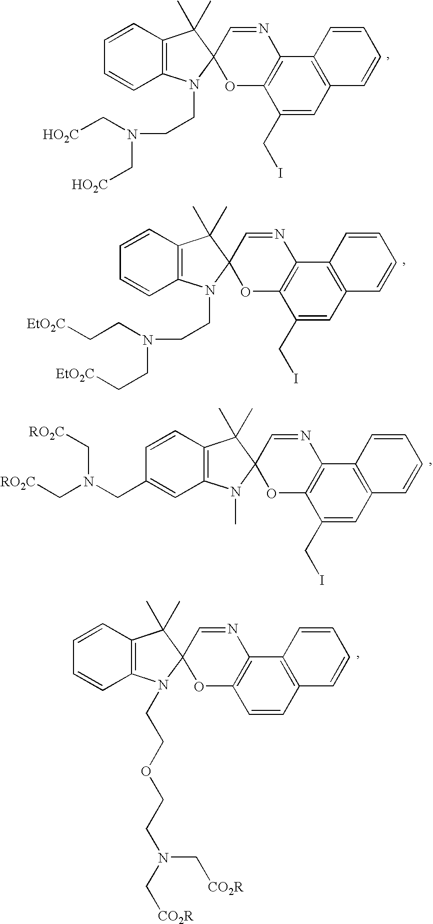 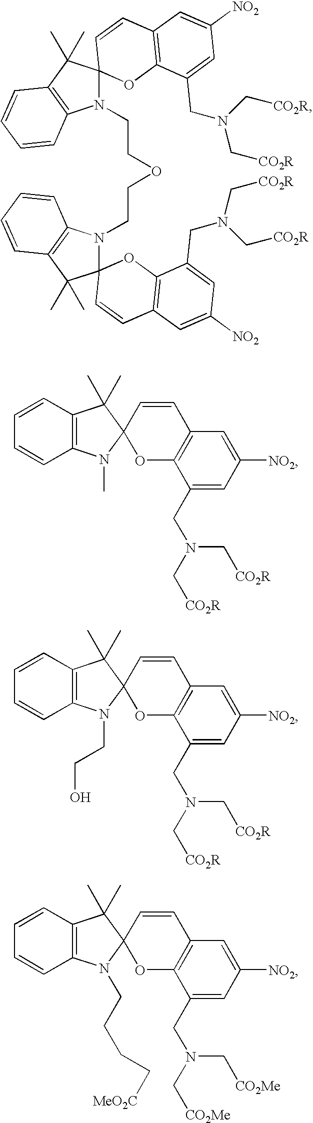 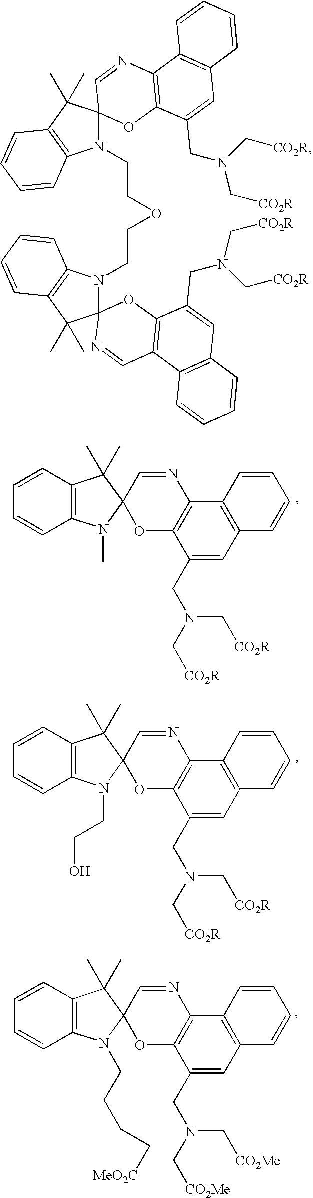 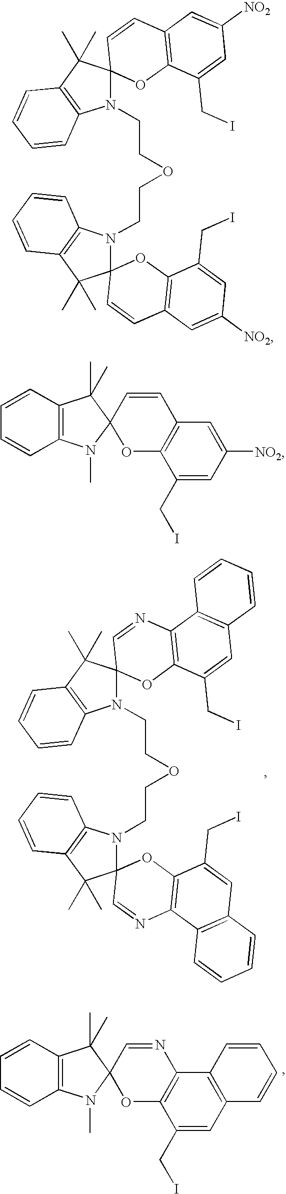 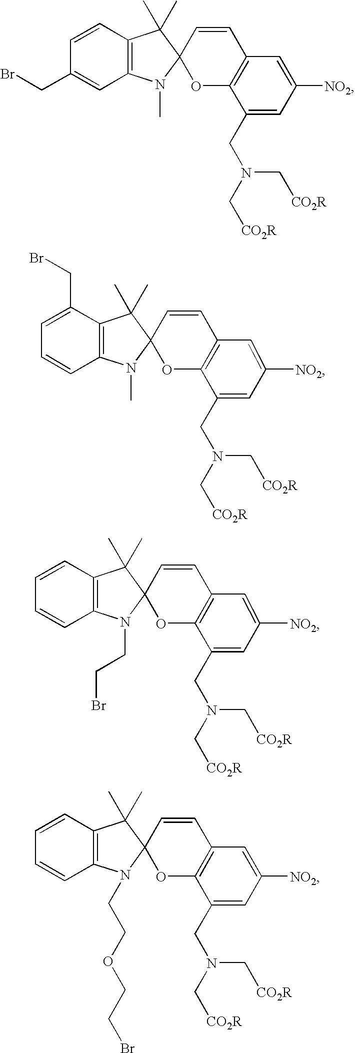 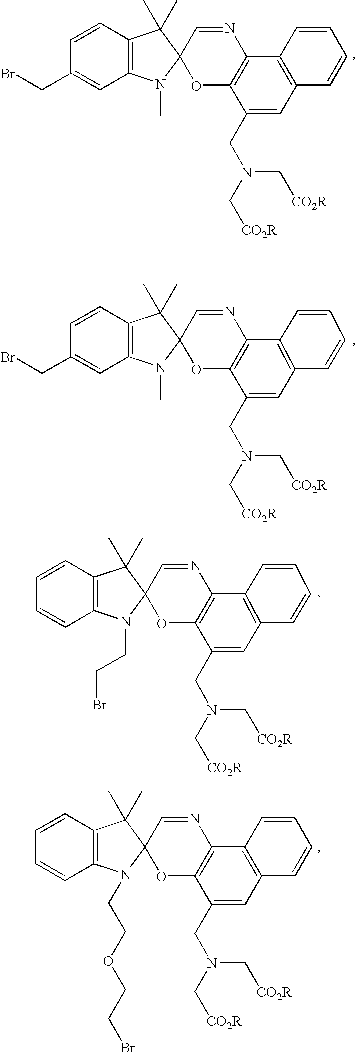 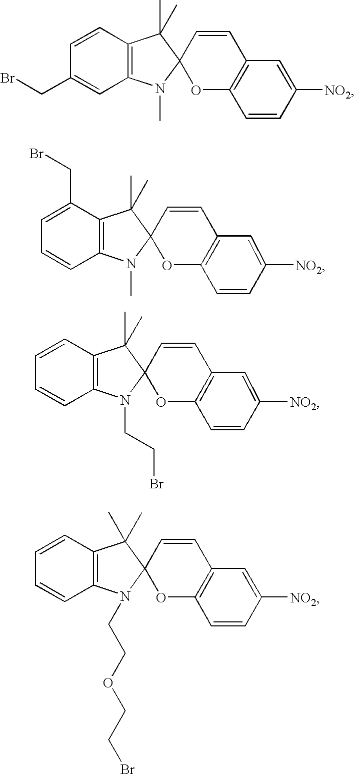 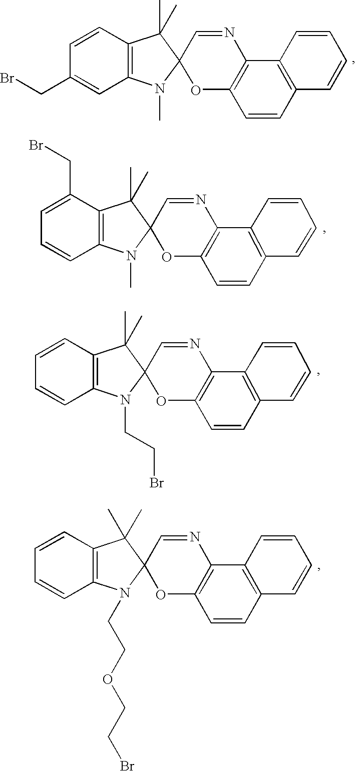 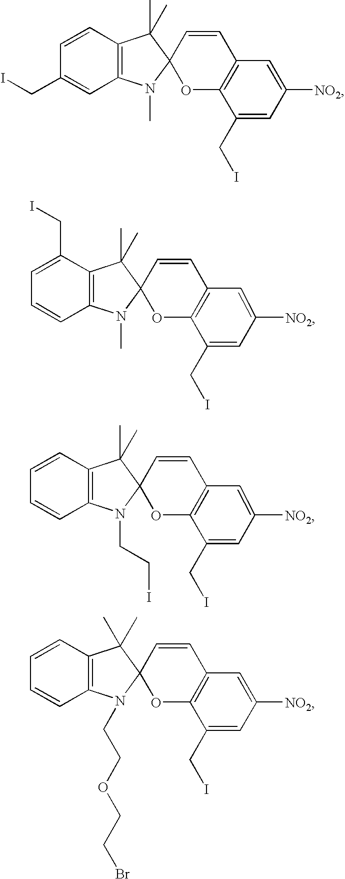 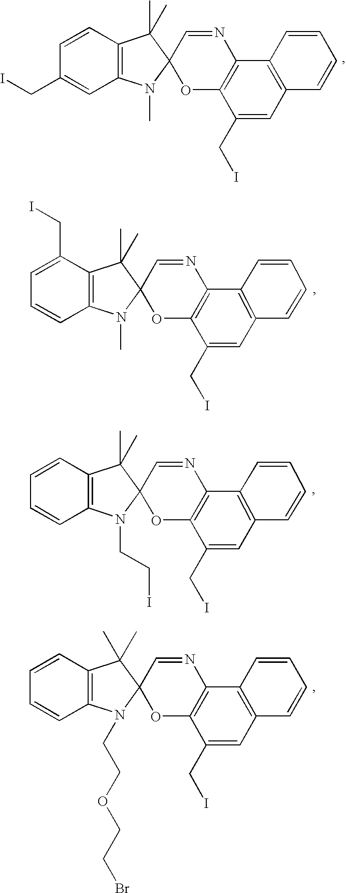 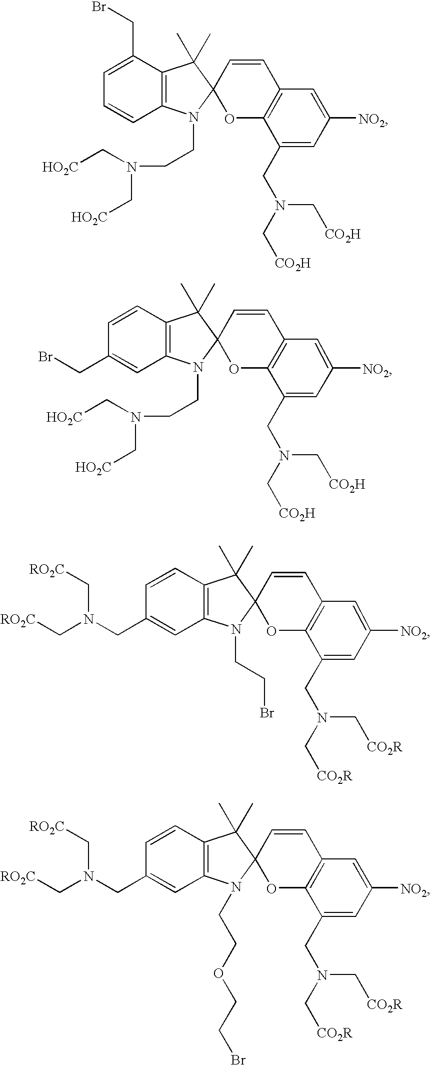 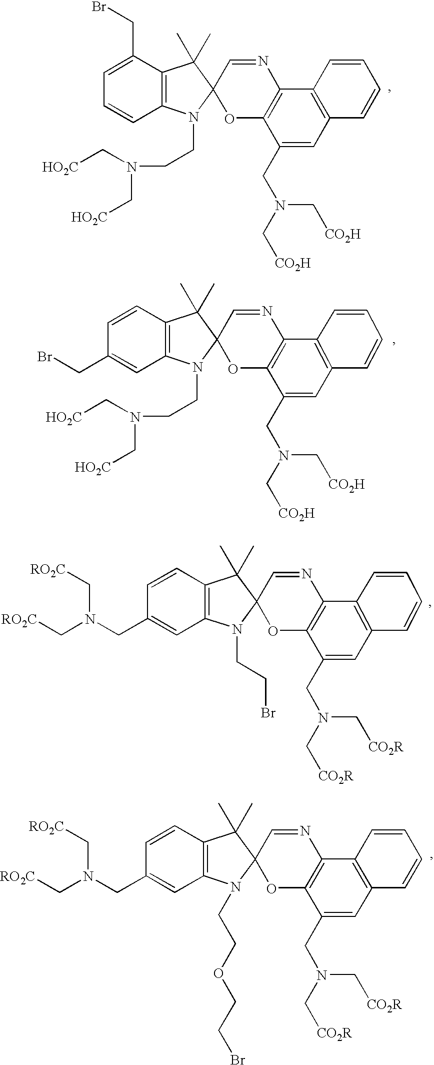 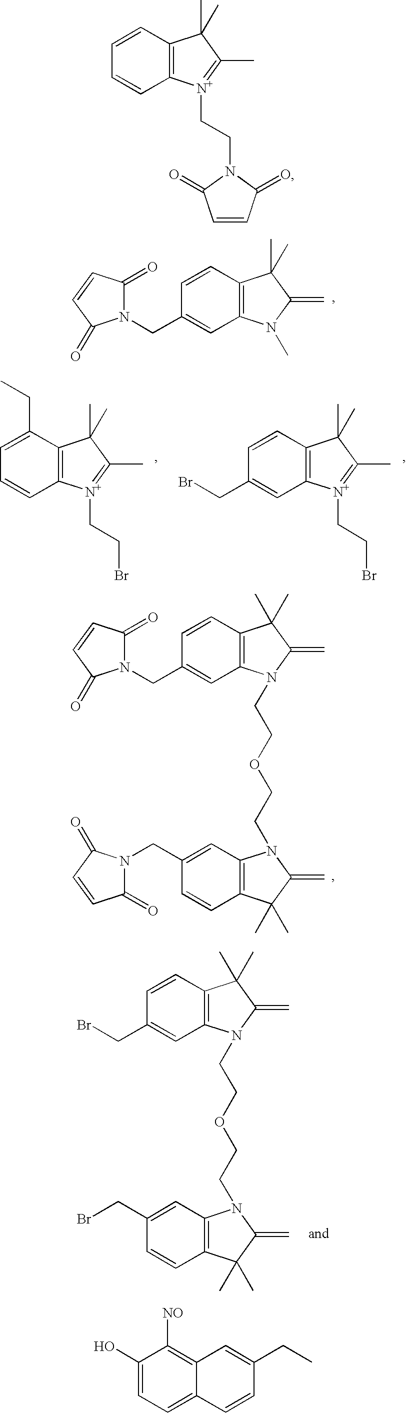
wherein R is independently selected from H, CH₃, C₂H₅ and C₃H₇.

2. A reversible optical photochromic probe comprising a compound of claim 1, wherein the probe is capable of undergoing light directed reversible transition between a first state and a second state.

3. A reversible optical photochromic probe of claim 2, wherein the first state is obtained by shining light of about 365 nm on the compound or derivative thereof.

4. A reversible optical photochromic probe of claim 2, wherein the second state is obtained by shining light of about 545 nm to 620 nm on the compound or derivative thereof.

5. A method of determining or controlling biomolecular interactions or activity comprising the step of contacting said biomolecule with an optical photochromic probe of a compound of claim 1, wherein the optical photochromic probe is capable of undergoing light directed reversible transition.

6. A method of determining or controlling biomolecular interactions or activity according to claim 5 wherein the biomolecular interactions are studied using Foerster resonance energy transfer (FRET), fluorescence recovery after photobleaching (FRAP), photoactivation of fluorescence (PAF) technologies and Speckle microscopy.

7. A method of determining or controlling biomolecular interactions or activity according to claim 5 wherein the optical photochromic probe is capable of undergoing light directed reversible transition between a first and second state.

8. A method of determining or controlling biomolecular interactions or activity according to claim 7 wherein the first state is obtained by shining light of about 365 nm on the compound or derivative thereof.

9. A method of studying or controlling biomolecular interactions or activity according to claim 7 wherein the second state is obtained by shining light of about 545 nm to 620 nm on the compound or derivative thereof.

10. A method of studying or controlling biomolecular interactions or activity according to claim 5 wherein the biomolecules are proteins, DNA, RNA, sugars, or ligands.

11. A method of determining free or bound calcium or controlling calcium binding in a subject, said method comprising the step of contacting the subject with a reversible optical photochromic probe of a compound of claim 1.

12. A method of determining free or bound calcium or controlling calcium binding of claim 11 wherein free or bound calcium determination or calcium binding is controlled by light directed reversible transition between a first state and a second state.

13. A method of determining free or bound calcium or controlling calcium binding of claim 11 wherein the calcium estimation and controlling calcium binding interactions are determined using pcFRET technology.

14. A method of determining free or bound calcium or controlling calcium binding of claim 12, wherein the optical photochromic probe has at least two optical switches, wherein each optical switch is independently controlled by light directed reversible transition between the first state and the second state.

15. A method of determining free or bound calcium or controlling calcium binding of claim 12 wherein the first state is obtained by shining light of about 365 nm on the compound or derivative thereof.

16. A method of determining free or bound calcium or controlling calcium binding of claim 12 wherein the second state is obtained by shining light of about 545 nm to 620 on the compound or derivative thereof.

17. A method of synthesizing a thiol reactive optical switch, comprising the steps of: (a) coupling an indoline derivative with a salycilaldehyde or nitrosonaphthol derivative to yield a spirobenzopyran or a spironaphthoxazine; and (b) conducting a halogen exchange reaction or bromination of alcohol or modified Mitsunobu reaction on the spirobenzopyran or spironaphthoxazine to yield a thiol reactive spirocompound useful as an optical switch.

18. A method of synthesizing a thiol reactive optical switch according to claim 17, wherein the indoline derivative is a compound selected from the group consisting of: 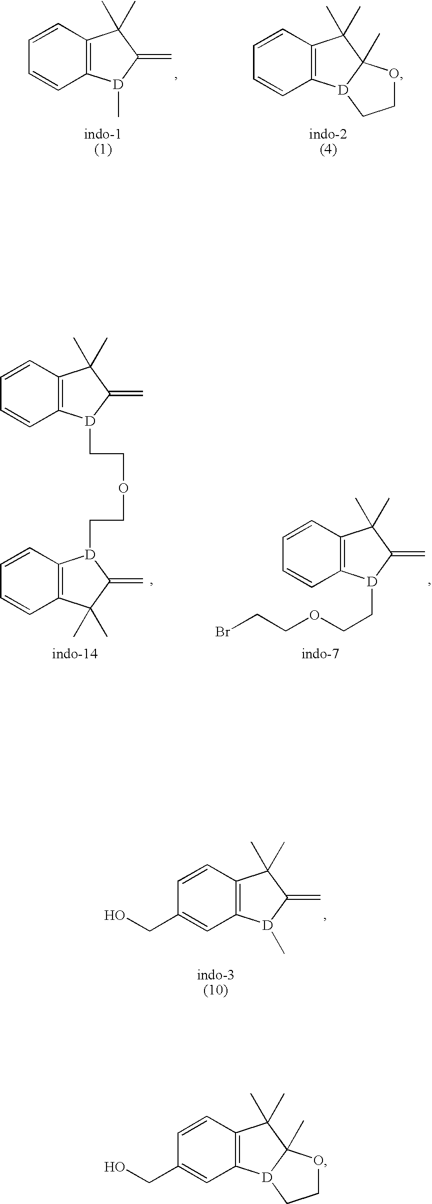 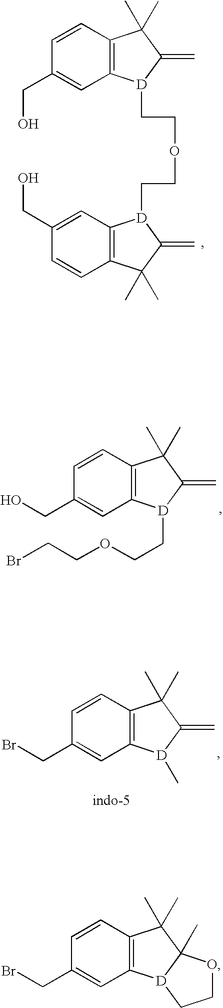 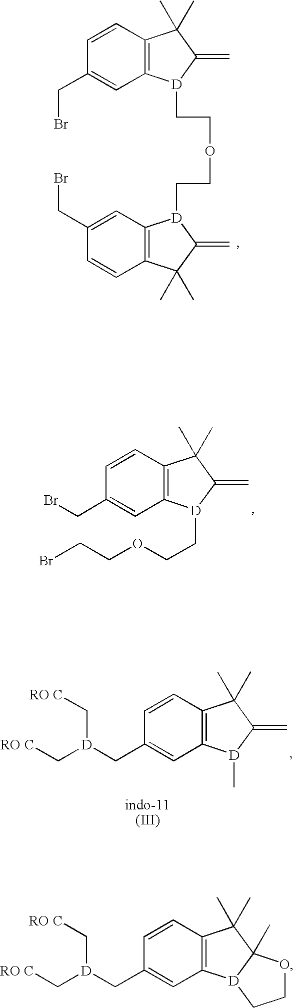 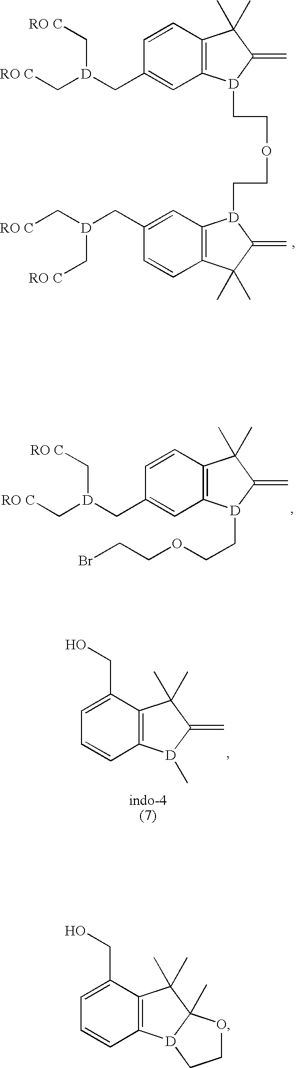 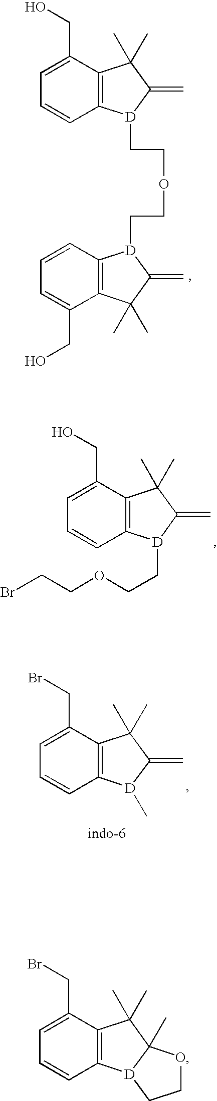 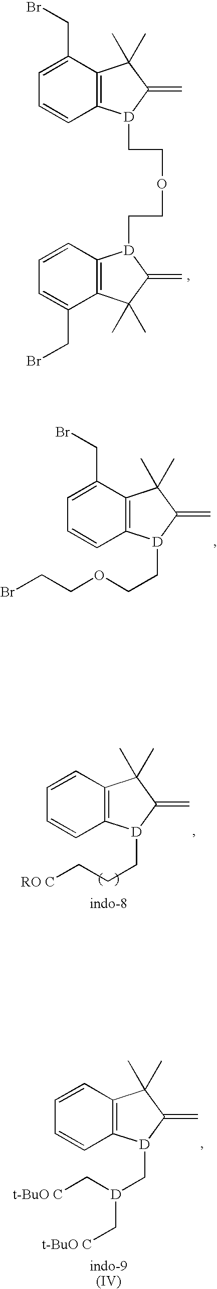 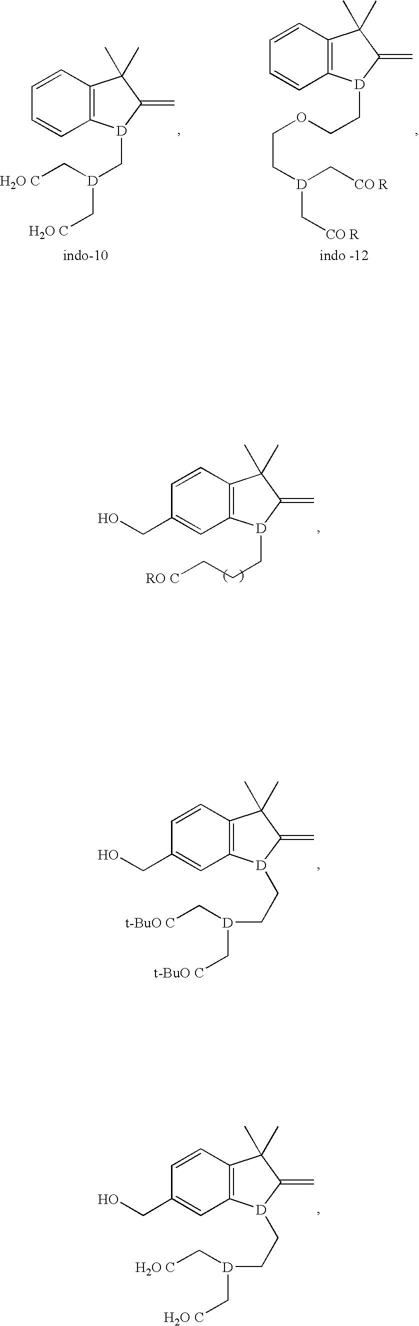 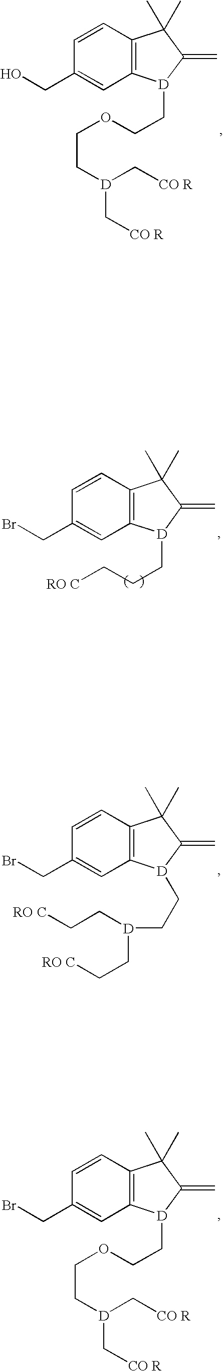 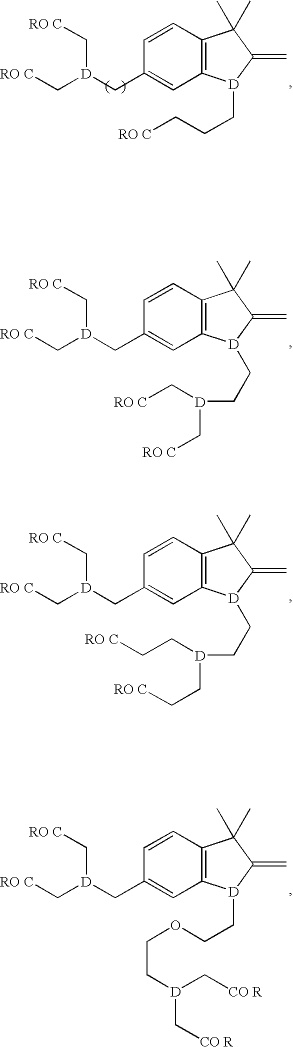 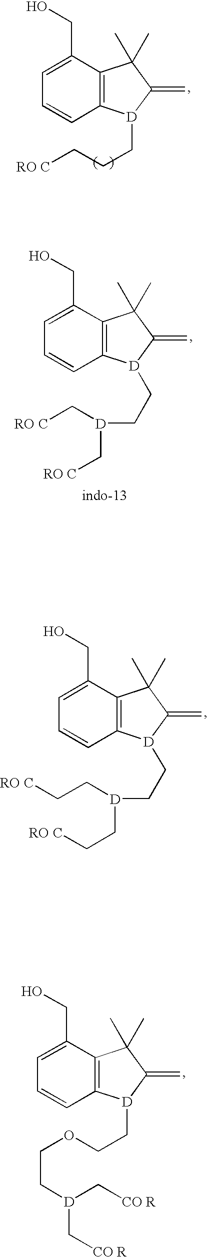 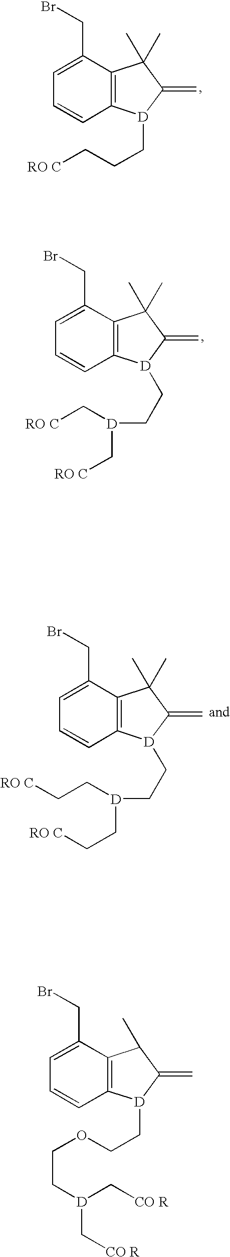
wherein R is independently selected from H, CH₃, C₂H₅ and C₃H₇.

19. A method of synthesizing a thiol reactive optical switch according to claim 17, wherein the spirobenzopyran or the spironaphthoxazine is a compound or a derivative thereof as shown in claim 1.

20. A method of synthesizing a thiol reactive optical switch according to claim 18, wherein the indoline derivative is synthesized by a coupling reaction of an indole derivative and an alkyl halide.
